(19) United States
(12) Patent Application Publication
(10) Pub. No.: US 2025/0082635 A1
(43) Pub. Date: Mar. 13, 2025

(54) METHOD OF TREATING DEPRESSION USING SELTOREXANT

(71) Applicant: Janssen Pharmaceutica NV, Beerse (BE)

(72) Inventors: Damayanthi Devineni, Raritan, NJ (US); Lingjue Li, Titusville, NJ (US)

(21) Appl. No.: 18/954,985

(22) Filed: Nov. 21, 2024

Related U.S. Application Data
(63) Continuation of application No. PCT/IB2024/056341, filed on Jun. 28, 2024.
(60) Provisional application No. 63/524,198, filed on Jun. 29, 2023.

(51) Int. Cl. A61K 31/506

(52) U.S. Cl. CPC A61K 31/506

(57) ABSTRACT

A method of treating an orexin-2 mediated disorder in a human subject in need thereof is provided. The method comprises administering seltorexant or a pharmaceutically acceptable salt or hydrate thereof at a therapeutically effective daily dose to the subject, when the subject does not have a hepatic impairment. The method also comprises administering seltorexant or the pharmaceutically acceptable salt or hydrate thereof at a reduced dose less than the daily dose to the subject, when the subject has moderate hepatic impairment.

### CROSS-REFERENCE TO RELATED APPLICATIONS

This application is a continuation of International Patent Application No. PCT/IB2024/056341, filed Jun. 28, 2024, which claims the benefit of the priority of U.S. Provisional Patent Application No. 63/524,198, filed Jun. 29, 2023, the disclosures of which are incorporated by reference herein.

### FIELD OF INVENTION

The present invention relates to methods for treating and/or preventing depression comprising administering to a subject in need thereof a therapeutically effective amount of an orexin-2 receptor antagonist, specifically, seltorexant.

### BACKGROUND OF INVENTION

Orexins (also known as hypocretins) are neuropeptides expressed by neurons in the perifornical area, the dorsomedial hypothalamus and the lateral hypothalamus (de Lecea et al., 1998; Proc. Natl. Acad. Sci. U.S.A. 95, 322-327; Sakaurai et al, 1998, Cell 92, 573-585). Orexinergic neurons project to many areas of the brain including other hypothalamic nuclei, the midline paraventricular thalamus, brain stem nuclei, the ventral tegmental area and nucleus accumbens shell. (Peyron et al., 1998, J. Neurosci. 18, 9996-10016) Orexin neuropeptides, classified as either orexin-A or orexin-B, bind to the seven transmembrane G-protein coupled receptors orexin-1 (OX1R) and orexin-2 (OX2R) (de Lecea et al., 1998; Proc. Natl. Acad. Sci. U.S.A. 95, 322-327; Sakaurai et al, 1998, Cell 92, 573-585). While orexin-A is non-selective for OX1R and OX2R, orexin-B shows higher affinity for OX2R (Sakaurai et al, 1998, Cell 92, 573-585). Orexin receptor antagonists are classified as single orexin receptor (SORAs) or dual receptor antagonists (DORAs).

Hypothalamic orexinergic neurons expressing discharge during active wake, are virtually silent during non-rapid eye movement sleep and show transient discharges during rapid eye movement sleep (Lee, 2005, J. Neuroscience 25(8): 6716-6720; Takahashi, 2008, Neuroscience, 153:860-870). This activity pattern supports the notion that the orexins are endogenous, potent, arousal (wakefulness)-promoting peptides. Studies using single unit recordings also show that OX-containing neurons are preferentially activated during rewarding appetitive behaviors (Hassani et al., 2016. J Neuroscience 36(5): 1747-1757).

### BRIEF SUMMARY OF THE INVENTION

In one aspect of the present application a method of treating an orexin-2 mediated disorder in a human subject in need thereof is provided. In some examples, the orexin-2 mediated disorder may include, but is not limited to, depression and insomnia. The depression may include, but is not limited to, major depressive disorder, bipolar depression or concurrent depression and insomnia symptoms having an ISI score of greater than or equal to 15. The method comprises administering seltorexant or a pharmaceutically acceptable salt or hydrate thereof (e.g., as a free base or a hydrochloride salt) at a therapeutically effective daily dose to the subject, when the subject does not have a hepatic impairment. The method also comprises administering seltorexant or the pharmaceutically acceptable salt or hydrate thereof at a reduced dose less than the daily dose to the subject, when the subject has moderate hepatic impairment (e.g., a Child-Pugh score from 7 to 9). In some examples, the method may further comprise administering seltorexant or a pharmaceutically acceptable salt or hydrate thereof at the daily dose to the subject, when the subject has mild hepatic impairment (e.g., a Child-Pugh score from 5 to 6). In additional examples, the method may also further comprise not administering seltorexant or a pharmaceutically acceptable salt or hydrate to the subject, when the subject has severe hepatic impairment (e.g., a Child-Pugh score at or above 10).

For treatment of depression (e.g. major depressive disorder, bipolar depression or concurrent depression and insomnia symptoms having an ISI score of greater than or equal to 15) administration of seltorexant or the pharmaceutically acceptable salt or hydrate thereof at the daily dose provides a first unbound average plasma concentration of seltorexant within an effective therapeutic range, when the subject does not have a hepatic impairment, and at the reduced dose provides a second unbound average plasma concentration of seltorexant within the effective therapeutic range, when the subject has moderate hepatic impairment. Additionally, administration of seltorexant or the pharmaceutically acceptable salt or hydrate thereof at the daily dose may provide a third unbound average plasma concentration of seltorexant within the effective therapeutic range, when the subject has mild hepatic impairment. In some examples, the administration of seltorexant or the pharmaceutically acceptable salt or hydrate thereof at the reduced dose is more therapeutically effective than the daily dose in the subject, when the subject has the moderate hepatic impairment.

In some examples, the subject may have an inadequate response to an antidepressants other than seltorexant or a pharmaceutically acceptable salt or hydrate thereof. In particular, the subject may have an inadequate response to a selective serotonin reuptake inhibitor (SSRI) or a serotonin and noradrenaline reuptake inhibitor (SNRI). In another example, seltorexant or a pharmaceutically acceptable salt or hydrate thereof may be adjunctively administered with a second pharmaceutically active agent. The second pharmaceutically active agent may be a selective serotonin reuptake inhibitor (SSRI) or a serotonin and noradrenaline reuptake inhibitor (SNRI).

In another aspect of the present application, a method of treating an orexin-2 mediated disorder in a human subject having moderate hepatic impairment (e.g., a Child-Pugh score from 7 to 9) is provided. The method comprises administering seltorexant or a pharmaceutically acceptable salt or hydrate thereof (e.g., as a free base or a hydrochloride salt) at a reduced dose that is less than a therapeutically effective daily dose adapted for treatment of a human subject having normal hepatic function. In some examples, the orexin-2 mediated disorder may include, but is not limited to, depression and insomnia. The depression may include, but is not limited to, major depressive disorder, bipolar depression or concurrent depression and insomnia symptoms having an ISI score of greater than or equal to 15.

In a further aspect of the present application, a method of treating an orexin-2 mediated disorder in a human subject having mild hepatic impairment (e.g., a Child-Pugh score from 5 to 6) is provided. The method comprises administering seltorexant or a pharmaceutically acceptable salt or hydrate thereof (e.g., as a free base or a hydrochloride salt) at a therapeutically effective daily dosage amount adapted for treatment of a human subject having normal hepatic function. In some examples, the orexin-2 mediated disorder may include, but is not limited to, depression and insomnia. The depression may include, but is not limited to, major depressive disorder, bipolar depression or concurrent depression and insomnia symptoms having an ISI score of greater than or equal to 15.

Another method of treating an orexin-2 mediated disorder in a human subject in need thereof is provided by the present application. If the subject has moderate hepatic impairment (e.g., a Child-Pugh score from 7 to 9), the method administers seltorexant or a pharmaceutically acceptable salt or hydrate thereof at a reduced dose that is less than a therapeutically effective daily dose adapted for treatment of a human subject having normal hepatic function. If the subject has severe hepatic impairment (e.g., a Child-Pugh score at or above 10), the method administers seltorexant or a pharmaceutically acceptable salt or hydrate to the subject. If the subject does not have moderate to severe hepatic impairment, the method administers seltorexant or a pharmaceutically acceptable salt or hydrate thereof at the daily dose to the subject. In some examples, the orexin-2 mediated disorder may include, but is not limited to, depression and insomnia. The depression may include, but is not limited to, major depressive disorder, bipolar depression or concurrent depression and insomnia symptoms having an ISI score of greater than or equal to 15.

Seltorexant may be administered via any pharmaceutically suitable routes, in particular, seltorexant may be orally administered. In some examples, the daily dose is about 20 mg/day to about 60 mg/day. The reduced dose may be about 5 mg/day to about 20 mg/day. The reduced dose maybe at least 20% less, at least 25% less or about 50% less than the daily dose. In another example, the daily dose is about 0.25 mg/kg per day to about 0.9 mg/kg per day, and the reduced dose is about 0.07 mg/day to about 0.3 mg/day. In a further example, the daily dose is selected such that administration of seltorexant or the pharmaceutically acceptable salt or hydrate thereof at the daily dose to the subject provides an unbound average plasma concentration of seltorexant between about 0.75 ng/mL and about 1.5 ng/mL, when the subject does not have a hepatic impairment.

For any of the aspects or embodiments described herein which relate to methods of treatment e.g., methods of treating an orexin-2 mediated disorder wherein said method comprises administering a compound or composition described herein, also provided by the present disclosure is said compound or composition for use in such methods.

These and other aspects of the invention will become apparent to those skilled in the art after a reading of the following detailed description of the invention, including the figures and appended claims.

### BRIEF DESCRIPTION OF THE DRAWINGS

FIGS. 1a and 1b show linear mean and semi-logarithmic mean plasma concentration-time profiles of seltorexant after oral administration of 20 mg seltorexant in Cohort 1 and Cohort 2, and 10 mg seltorexant in Cohort 3 according to Example 1.

FIGS. 2a and 2b show linear mean and semi-logarithmic mean plasma concentration-time profiles of unbound seltorexant after oral administration of 20 mg seltorexant in Cohort 1 and Cohort 2, and 10 mg seltorexant in Cohort 3 according to Example 1.

FIGS. 3a through 3e show scatter plots of seltorexant AUCₗₐₛₜ, AUC_{∞} against α-1-AGP levels, CL/F against total protein, and Cₘₐₓ against α-1-AGP levels after oral administration of 20 mg seltorexant in Cohort 1 and Cohort 2, and 10 mg Seltorexant in Cohort 3 (dose normalized to 20 mg) according to Example 1.

FIGS. 4a through 4k show scatter plots of unbound seltorexant C_{max,unbound}, AUC_{last,unbound} and AUC_{∞,unbound} against albumin, prothrombin time, ALP and α-1-AGP levels after oral administration of 20 mg seltorexant in Cohort 1 and Cohort 2, and 10 mg seltorexant in Cohort 3 (dose normalized to 20 mg) according to Example 1.

FIGS. 5a and 5b show linear mean and semi-logarithmic mean plasma concentration-time profiles of M12 after oral administration of 20 mg seltorexant in Cohort 1 and Cohort 2, and 10 mg seltorexant in Cohort 3 according to Example 1.

FIGS. 6a and 6b show linear mean and semi-logarithmic mean plasma concentration-time profiles of unbound M12 after oral administration of 20 mg seltorexant in Cohort 1 and Cohort 2, and 10 mg seltorexant in Cohort 3 according to Example 1.

FIGS. 7a through 7d show scatter plots of M12 AUCₗₐₛₜ, AUC_{∞} against α-1-AGP levels and Cₘₐₓ against total protein and α-1-AGP after oral administration of 20 mg seltorexant in Cohort 1 and Cohort 2, and 10 mg seltorexant in Cohort 3 (dose normalized to 20 mg) according to Example 1.

FIGS. 8a through 8f show scatter plots of unbound M12 AUC_{last,unbound}, AUC_{∞,unbound}, against ALP and α-1-AGP Levels and C_{max,unbound}, against prothrombin time and α-1-AGP Levels after oral administration of 20 mg seltorexant in Cohort 1 and Cohort 2, and 10 mg seltorexant in Cohort 3 (dose normalized to 20 mg) according to Example 1.

FIGS. 9a and 9b show linear mean and semi-logarithmic mean plasma concentration-time profiles of M16 after oral administration of 20 mg seltorexant in Cohort 1 and Cohort 2, and 10 mg seltorexant in Cohort 3 according to Example 1.

FIGS. 10a and 10b show linear mean and semi-logarithmic mean plasma concentration-time profiles of unbound M16 after oral administration of 20 mg seltorexant in Cohort 1 and Cohort 2, and 10 mg seltorexant in Cohort 3 according to Example 1.

FIGS. 11a through 11c show scatter plots of M16 Cₘₐₓ, AUCₗₐₛₜ and AUC_{∞}, against α-1-AGP levels after oral administration of 20 mg seltorexant in Cohort 1 and Cohort 2, and 10 mg seltorexant in Cohort 3 according to Example 1.

FIGS. 12a through 12h show scatter plots of Unbound M16 AUC_{last,unbound} and AUC_{∞,unbound}, against albumin, prothrombin time, α-1-AGP levels and C_{max,unbound} against prothrombin time, α-1-AGP levels, after oral administration of 20 mg seltorexant in Cohort 1 and Cohort 2, and 10 mg seltorexant in Cohort 3 according to Example 1.

### DETAILED DESCRIPTION OF THE INVENTION

Unless defined otherwise, all technical and scientific terms used herein have the same meaning commonly understood to one of ordinary skill in the art to which this invention pertains. Otherwise, certain terms used herein have the meanings as set in the specification. All patents, published patent applications and publications cited herein are incorporated by reference as if set forth fully herein. It is noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps. When used herein the term “comprising” can be substituted with the term “containing” or “including” or sometimes when used herein with the term “having.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the application can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise stated, any numerical value, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1 mg/mL to 10 mg/mL includes 0.9 mg/mL to 11 mg/mL. More particularly, the numerical value may include ±1%, ±3%, or ±5% of the recited value. As used herein, the use of a numerical range expressly includes all possible subranges, all individual numerical values within that range, including integers within such ranges and fractions of the values unless the context clearly indicates otherwise.

The term “subject” or “patient” as used herein refers to an animal, and preferably a mammal. According to particular embodiments, the subject is a mammal including a non-primate (e.g., a camel, donkey, zebra, cow, pig, horse, goat, sheep, cat, dog, rat, rabbit, guinea pig, marmoset or mouse) or a primate (e.g., a monkey, chimpanzee, or human). In particular embodiments, the subject is a human.

The term “pharmaceutically acceptable salt” refers to a salt of any of the compounds herein which are known to be non-toxic and are commonly used in the pharmaceutical literature. In some embodiments, the pharmaceutically acceptable salt of a compound retains the biological effectiveness of the compounds described herein and are not biologically or otherwise undesirable. Suitable pharmaceutically acceptable salts of the compounds include acid addition salts which may, for example, be formed by mixing a solution of the compound with a solution of a pharmaceutically acceptable acid such as for example, inorganic acids such as hydrohalic acids, e.g., hydrochloric or hydrobromic acid, sulfuric, nitric, phosphoric and the like acids; or organic acids such as, for example, acetic, propanoic, hydroxyacetic, lactic, pyruvic, oxalic (i.e., ethanedioic), malonic, succinic (i.e., butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

The desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid, glutaric acid, or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

As used herein, the terms “treat,” “treating,” and “treatment” are all intended to refer to an amelioration or reversal of at least one measurable physical parameter related to a disease, disorder, or conduction, such as, for example, an orexin-2 mediated disorder, disease or condition, including depression (e.g., major depressive disorder, bipolar depression) or insomnia. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with depression (e.g., major depressive disorder, bipolar depression) or insomnia. The one or more symptoms of depression include psychiatric symptoms of depression, such as, depressed mood (e.g., feels sad, empty, hopeless), irritable mood, diminished interest or pleasure in all, or almost all, activities, fatigue or loss of energy, feelings of worthlessness or excessive or inappropriate guilt, diminished ability to think or concentrate, and indecisiveness. In some examples, the psychiatric symptoms of depression may be concurrent with insomnia symptoms having an Insomnia Severity Index (ISI) score of greater than or equal to 15.

ISI is a 7-item questionnaire assessing nature, severity, impact of insomnia. The dimensions evaluated by the ISI are: severity of sleep onset, sleep maintenance, early morning awakening problems; sleep dissatisfaction; interference of sleep problem with daytime functioning; noticeability of sleep problems; distress caused by sleep difficulties. A 5-point Likert scale (0-4) is used to rate each item. Scores for the 7-items are summed for a total score which ranges from 0-28. Negative changes in scores indicate improvement. ISI total score is the sum of all item scores. Total score interpreted insomnia as follows: absence (0-7); sub-threshold (8-14); moderate (15-21); severe (22-28).

In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

One skilled in the art will recognize that wherein methods of prevention are described, a subject in need thereof (i.e., a subject in need of prevention) shall include any subject who has experienced or exhibited at least one symptom of the disorder, disease or condition to be prevented. Further, a subject in need thereof may additionally be a subject (preferably a mammal, more preferably a human) who has not exhibited any symptoms of the disorder, disease or condition to be prevented, but who has been deemed by a physician, clinician or other medical profession to be at risk of developing said disorder, disease or condition. For example, the subject may be deemed at risk of having new episodes of depression (and therefore in need of secondary prevention or preventive treatment) as a consequence of the subject's medical history, including, but not limited to, family history, pre-disposition, co-existing (comorbid) disorders or conditions, genetic testing, and the like.

The term “therapeutically effective amount” as used herein refers to that amount of active compound or pharmaceutical agent that elicits the biological or medicinal response in a tissue system, animal or human that is being sought by a researcher, veterinarian, medical doctor or other clinician, which includes alleviation of one or more of the symptoms of the disease or disorder being treated; and/or reduction of the severity of one or more of the symptoms of the disease or disorder being treated.

The term “antagonist” as used herein refers to a compound that binds to a receptor that blocks or attenuates the receptor's response to an agonist.

As used herein, the term “depression” encompasses any mood disorder that is defined as a “depressive disorder” by the Diagnostic and Statistical Manual for Mental Disorders (DSM-5). The term “depression” includes major depressive disorder (MDD), persistent depressive disorder, seasonal affective disorder, psychotic depression, postpartum depression, premenstrual dysphoric disorder, situational depression, anhedonia, melancholy, mid-life depression, late-life depression, depression due to identifiable stressors, or combinations thereof. The term “depression” may also include bipolar depression, which is the depression associated with, characteristic of, or symptomatic of a bipolar disorder. In certain embodiments, the depression is MDD. In other embodiments, the MDD is with melancholic features or anxious distress.

The present application is directed to methods for treating and/or preventing an orexin-2 mediated disorder, disease or condition in a human subject in need thereof by administering a therapeutically effective amount of a selective orexin-2 antagonist. In particular, the orexin-2 antagonist is [5-(4,6-dimethyl-pyrimidin-2-yl)-hexahydro-pyrrolo[3,4-c]pyrrol-2-yl]-(2-fluoro-6-[1,2,3]triazol-2-yl-phenyl)-methanone having the structure of Formula I,

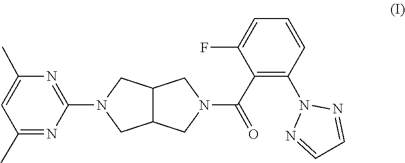 [SMILES: Cc1cc(C)nc(N2CC3CN(C(=O)c4c(F)cccc4-n4nccn4)CC3C2)n1]

(also known as seltorexant) or a pharmaceutically acceptable salt or hydrate thereof. In one embodiment, the compound administered is [5-(4,6-dimethyl-pyrimidin-2-yl)-hexahydro-pyrrolo[3,4-c]pyrrol-2-yl]-(2-fluoro-6-[1,2,3]triazol-2-yl-phenyl)-methanone having the structure of Formula I, as a free base. In another embodiment, the compound administered is [5-(4,6-dimethyl-pyrimidin-2-yl)-hexahydro-pyrrolo[3,4-c]pyrrol-2-yl]-(2-fluoro-6-[1,2,3]triazol-2-yl-phenyl)-methanone hydrochloride. Suitable selective orexin-2 antagonists for the methods described herein are described in PCT Patent Application Publication No. WO2011/050198, U.S. Pat. No. RE48,841, U.S. Pat. Nos. 9,079,911, 11,059,828, and 11,667,644, which are all incorporated by reference herein. An improved synthetic method for making fused heterocyclic compounds, such as seltorexant, as orexin receptor modulators is described in PCT Patent Application Publication No. WO2020/100011, which is also incorporated by reference herein.

The orexin-2 mediated disorder being treated and/or prevented by compounds described herein include, but is not limited to any one of depressive disorders, bipolar and related disorders, and sleep-wake disorders as defined in DSM-5. In particular, the orexin-2 mediated disorder may be selected from a group consisting of depression (e.g., MDD, bipolar depression) and insomnia. In one embodiment, the compounds described herein are administered to a human subject for the treatment and/or prevention of MDD. In another embodiment, the compounds described herein are administered to a human subject for reducing one or more symptoms of depression. In a further embodiment, the compounds described herein are administered to a human subject for the treatment and/or prevention of bipolar depression, i.e., depressive episodes associated bipolar and related disorders as defined in DSM-5 (e.g., bipolar I disorder, bipolar II disorder, or cyclothymic disorder). In another embodiment, the compounds described herein are administered to a human subject for the treatment and/or prevention of insomnia. In a further embodiment, the compounds described herein are administered for treating and/or preventing depression in a human subject having concurrent depression and insomnia symptoms having an ISI score of greater than or equal to 15. Additionally, the compound of Formula (I) may also be in the form of solvates, and polymorphs, and mixtures thereof. The compound of Formula (I) or pharmaceutically acceptable salts of the compound of Formula (I) may be obtained as solvates. Solvates include those formed from the interaction or complexation of a compound with one or more solvents, either in solution or as a solid or crystalline form. In some embodiments, the solvent is water and then the solvates are hydrates. In addition, crystalline forms of the compound of Formula (I) or pharmaceutically acceptable salts of a compound of Formula (I) may be obtained as co-crystals. In certain embodiments, the compound of Formula (I) is obtained in a crystalline form. In other embodiments, a crystalline form of the compound of Formula (I) is cubic in nature. In other embodiments, pharmaceutically acceptable salts of the compound of Formula (I) are obtained in a crystalline form. In still other embodiments, the compound of Formula (I) is obtained in one of several polymorphic forms, as a mixture of crystalline forms, as a polymorphic form, or as an amorphous form. In other embodiments, the compound of Formula (I) converts in solution between one or more crystalline forms and/or polymorphic forms.

Patients who have moderate to severe hepatic impairment may have a reduced ability to metabolize and/or eliminate a drug as compared to a patient having normal hepatic function. Often, those patients with moderate to severe hepatic impairment are prevented from using the drug if there is a significant change in the pharmacokinetic profile or parameter(s) (e.g., maximum plasma concentration (Cₘₐₓ), average plasma concentration (C_{avg}) and/or area under the plasma concentration-time curve (AUC)) of the drug. The change in pharmacokinetic profile or parameter(s) can result in increased toxicity and/or changes to the efficacy of the drug. Therefore, patients with moderate to severe hepatic impairment are often excluded from eligible treatment populations. Such an exclusion removes the drug from being available as a treatment option for patients with moderate to severe hepatic impairment. The methods of the present application provides a way for patients having moderate hepatic impairment, who may have a reduced ability to metabolize and/or eliminate seltorexant, to be administered a therapeutically effective amount of seltorexant or a pharmaceutically acceptable salt or hydrate thereof. In some examples, the methods of the present application provides a way for patients having moderate hepatic impairment to be administered seltorexant or a pharmaceutically acceptable salt or hydrate thereof, while maintaining the pharmacokinetic profile or parameter(s) of seltorexant in such patients having the moderate hepatic impairment within an effective therapeutic range. Patients with severe hepatic impairment may still be prevented from being administered seltorexant or a pharmaceutically acceptable salt or hydrate thereof.

Seltorexant is primarily metabolized into a compound having the structure of Formula II (hereinafter referred as “M12” metabolite),

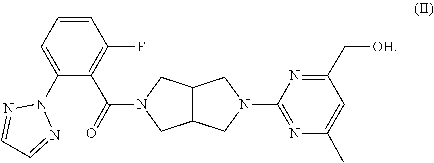 [SMILES: Cc1cc(CO)nc(N2CC3CN(C(=O)c4c(F)cccc4-n4nccn4)CC3C2)n1]

It is also metabolized into a compound having the structure of Formula III (hereinafter referred as “M16” metabolite),

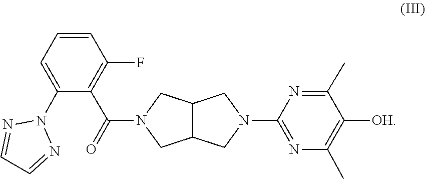 [SMILES: Cc1nc(N2CC3CN(C(=O)c4c(F)cccc4-n4nccn4)CC3C2)nc(C)c1O]

Although M12 is an active metabolite, the contribution of M12 to efficacy of seltorexant or a pharmaceutically acceptable salt or hydrate thereof is estimated to be about 18% to about 26% activity. However, in some examples, it may be desirable to maintain plasma concentration of the M12 metabolite within a desired range. The M16 metabolite is also an active metabolite, but it is a minor metabolite which contributes to <4% activity. However, in some examples, it may be desirable to maintain plasma concentration of the M16 metabolite within a desired range.

Patients who have moderate hepatic impairment may have a reduced ability to metabolize seltorexant into M12 and M16 and/or eliminate seltorexant as compared to a patient having normal hepatic function. Therefore, in patients that have moderate hepatic impairment, seltorexant may have an altered pharmacokinetic profile or altered pharmacokinetic parameter(s) (e.g., maximum plasma concentration (Cₘₐₓ), average plasma concentration (C_{avg}) and/or area under the plasma concentration-time curve (AUC)) for seltorexant, the M12 metabolite and/or the M16 metabolite as compared to those patients having normal hepatic function administered with seltorexant in the same manner.

Specifically, when subjects having moderate hepatic impairment are administered a normal daily dosage amount of seltorexant or a pharmaceutically acceptable salt or hydrate thereof, a level of total and/or unbound exposure (e.g., concentration in plasma) of seltorexant and/or its active metabolites are higher as compared to the same normal daily dosage amount being administered to those subjects who do not have a hepatic impairment. The normal daily dosage amount is a daily dosage amount that is adapted for subjects who do not have a hepatic impairment (e.g., having normal hepatic function). Therefore, those subjects having moderate hepatic impairment need to be administered a lower dosage amount that is less than the normal daily dosage amount in order to achieve a pharmacokinetic profile or pharmacokinetic parameter(s) (e.g., maximum plasma concentration (Cₘₐₓ), average plasma concentration (C_{avg}) and/or area under the plasma concentration-time curve (AUC)) comparable to, similar to, or substantially the same as the pharmacokinetic profile or pharmacokinetic parameter(s) of the normal daily dosage amount in subjects who do not have a hepatic impairment.

In some examples, subjects having mild hepatic impairment are administered the same normal daily dosage amount of seltorexant or a pharmaceutically acceptable salt or hydrate thereof as subjects who do not have a hepatic impairment (e.g., having normal hepatic function). It is believed that although patients with mild hepatic impairment may have a reduced ability to metabolize and/or eliminate a drug as compared to a patient having normal hepatic function, such a reduction does not yield a significant change in the pharmacokinetic profile or parameter(s) (e.g., maximum plasma concentration (Cₘₐₓ), average plasma concentration (C_{avg}) and/or area under the plasma concentration-time curve (AUC)) of seltorexant or its active metabolites. Therefore, it is believed that seltorexant or a pharmaceutically acceptable salt or hydrate thereof can be safely administered to subjects having mild hepatic impairment at the normal daily dosage amount adapted for subjects who do not have a hepatic impairment.

Seltorexant is believed to have a dose-dependent pharmacodynamic profile in treating and/or preventing an orexin-2 mediated disorder (e.g., treatment of insomnia) in human subjects. However, in the treatment of depression, clinical efficacy of seltorexant initially increases with increasing dosage, but reaches a plateau and subsequently decreases in clinical efficacy as the dosage is further increased. In particular, seltorexant is believed to have a dose-dependent pharmacodynamic profile in treating and/or preventing depression (e.g., MDD, bipolar depression) in human subjects, where clinical efficacy initially increases with increasing dosage, but reaches a plateau and subsequently decreases in clinical efficacy as the dosage is further increased. It was observed in a clinical trial (Clinical Trial Identifier: NCT03227224) that seltorexant administered at 20 mg/day showed greater efficacy than 10 mg/day (which showed no benefit) and a trend towards greater efficacy than 40 mg/day (which showed modest evidence of benefit) on mean change in MADRS scores. See Savitz et al., “Efficacy and Safety of Seltorexant as Adjunctive Therapy in Major Depressive Disorder: A Phase 2b, Randomized, Placebo-Controlled, Adaptive Dose-Finding Study,” International Journal of Neuropsychopharmacology, 24 (12): 965-976 (2021). In view of this clinical data, seltorexant is believed to be pharmacodynamically effective when exposure of seltorexant in plasma of a patient is maintained within an effective therapeutic range for treating and/or preventing an orexin-2 mediated disorder, in particular, depression (e.g., MDD, bipolar depression). The therapeutic range (inclusive of the endpoints) is a range from a minimum therapeutic threshold (e.g., a minimum plasma concentration), below which seltorexant does not have a pharmacodynamic effect, to a maximum therapeutic threshold (e.g., a maximum plasma concentration), above which seltorexant is not therapeutically effective for treating and/or preventing a disorder, disease, or condition such as, for example, an orexin-2 mediated disorder, including depression (e.g., MDD, bipolar depression). More specifically, the therapeutic range (inclusive of the endpoints) is a range from a minimum therapeutic threshold, below which seltorexant is not therapeutically effective for treating and/or preventing depression, to a maximum therapeutic threshold, above which seltorexant is not therapeutically effective for treating and/or preventing depression.

In some examples, when subjects having moderate hepatic impairment are administered a normal daily dosage amount of seltorexant or a pharmaceutically acceptable salt or hydrate thereof, the subject may have a level of total and/or unbound exposure (e.g., concentration in plasma) of seltorexant and/or its active metabolites that exceeds the therapeutic range. However, when the subjects having moderate hepatic impairment are administered a lower dosage amount compared to the normal daily dosage amount of seltorexant or a pharmaceutically acceptable salt or hydrate thereof, the subject may have a level of total and/or unbound exposure (e.g., concentration in plasma) of seltorexant and/or its active metabolites that is within the therapeutic range.

In one example, seltorexant or a pharmaceutically acceptable salt or hydrate thereof is administered to a subject having mild hepatic impairment at the normal daily dosage amount when the mild hepatic impairment does not result in a significant change to the pharmacokinetic profile or parameter(s) for seltorexant and/or its active metabolites. Therefore, in some examples, a subject having mild hepatic impairment may be administered the normal daily dosage and have a level of total and/or unbound exposure (e.g., concentration in plasma) of seltorexant and/or its active metabolites that is within the therapeutic range.

In another example, seltorexant or a pharmaceutically acceptable salt or hydrate thereof is administered to a subject having mild hepatic impairment at an amount less than the normal daily dosage amount (e.g., the same lower dosage amount administered to subjects having moderate hepatic impairment or a different dosage amount that is less than the normal dosage amount but greater than the amount for subjects having moderate hepatic impairment) when the mild hepatic impairment results in a significant change to the pharmacokinetic profile or parameter(s) for seltorexant and/or its active metabolites. Therefore, in other examples, a subject having mild hepatic impairment may be administered an amount less than the normal daily dosage amount (e.g., the same lower dosage amount administered to subjects having moderate hepatic impairment or a different dosage amount that is less than the normal dosage amount but greater than the amount for subjects having moderate hepatic impairment), and have a level of total and/or unbound exposure (e.g., concentration in plasma) of seltorexant and/or its active metabolites that is within the therapeutic range.

In a Phase 2 clinical trial, as described further in Example 1, in subject having mild hepatic impairment, the total area under the plasma concentration-time curve (AUC) for seltorexant in subjects having mild hepatic impairment has a 1.40 fold increase as compared to the total AUC for subjects having normal hepatic function. In addition, unbound AUC for seltorexant in subjects having mild hepatic impairment has a 1.68 fold increase as compared to the unbound AUC for subjects having normal hepatic function.

The methods of the present application comprise first determining whether a human subject has a hepatic impairment to determine at what dosage seltorexant or a pharmaceutically acceptable salt or hydrate thereof is to be administered to the subject. The determination may be made by any suitable means for assessing whether the subject has a hepatic impairment. For example, the determination may be made based on physical examination by a healthcare provider, data from laboratory tests conducted on the subject, and/or information in the medical records of the subject. In another example, the determination may be made by analyzing data from laboratory tests conducted on the subject, and/or information in the medical records of the subject to determine whether the subject has a hepatic impairment and/or severity of the hepatic impairment.

Severity of hepatic impairment in a subject may be quantified using any suitable clinical scale for quantitatively assessing prognosis of liver disease or cirrhosis, such as, for example, Child-Pugh score, MELD score, MELD-Na score, etc. In a particular example, hepatic impairment is quantified using the Child-Pugh score based on the parameters shown below in Table 2.

TABLE 2
Child-Pugh Classification

| Clinical and biochemical | Points scored for increasing abnormality | | |
|---|---|---|---|
| measurements | 1 | 2 | 3 |
| Encephalopathy (grade) | None | 1 or 2 | 3 or 4 |
| Ascites | Absent | Slight | Moderate |
| Bilirubin (mg/dL) | <2.0 | 2.0-3.0 | >3.0 |
| Albumin (g/dL) | >3.5 | 2.8-3.5 | <2.8 |
| International Normalization | <1.7 | 1.7-2.3 | >2.3 |
| Ratio (INR) | | | |

The sum of the scores for the five categories identified above are used to categorize the subject into one of the following categories:
Under 5 points: Normal hepatic function with no evidence of liver damage,
5 to 6 points: mild hepatic impairment (Child-Pugh A),
7 to 9 points: moderate hepatic impairment (Child-Pugh B), and
10 to 15 points: severe hepatic impairment (Child-Pugh C).
   It is noted that the Child-Pugh classification is applicable to exclusively hepatically impaired participants to characterize the degree of impairment of a proven chronic condition or cirrhosis of any pathology (misleadingly, healthy matches would have a score of 5 as with the mild hepatically impaired).

In one particular embodiment, prognosis of hepatic impairment of the subject is quantified using the Child-Pugh score. If the subject has a Child-Pugh score of 7 to 9 points, then the subject is administered the lower dosage amount of seltorexant or a pharmaceutically acceptable salt or hydrate thereof that is less than the normal daily dosage amount that is adapted for subjects who have normal hepatic function. Specifically, the normal daily dosage amount of seltorexant or a pharmaceutically acceptable salt or hydrate thereof is adapted for the treatment of those subjects with normal hepatic function (e.g., having a Child-Pugh score under 5). The lower dosage amount administered to those subjects having moderate hepatic impairment may be at least 20% less, at least 25% less, at least 30% less, at least 35% less, at least 40% less, about 50% less, at least 50% less, at least than 60%, at least 75% at least 80% less, or at least 90% less the normal daily dosage amount adapted for the treatment of those subjects with normal hepatic function. If the subject has mild hepatic impairment (e.g., a Child-Pugh score of 5 to 6 points), then the subject may be administered the normal daily dosage amount that is adapted for subjects with normal hepatic function.

The normal daily dosage amount in a subject without a reduced ability to metabolize or eliminate seltorexant (e.g., without hepatic impairment) may provide a maximum plasma concentration or an average plasma concentration within a therapeutic range required for pharmacodynamic effect. In some embodiments, a normal daily dosage amount is selected so that maximum plasma concentration (C_{max,normal}) or average plasma concentration (C_{avg,normal}) of seltorexant is within their respective therapeutic ranges required for pharmacodynamic effect in a subject without a reduced ability to metabolize or eliminate seltorexant (e.g., without hepatic impairment). The normal daily dosage amount, when administered to a subject with a hepatic impairment may provide a maximum plasma concentration (C_{max,impaired}) or an average plasma concentration (C_{avg,impaired}) that is less pharmacodynamically effective than C_{max,normal} or C_{avg, normal}. For example, C_{max,impaired} or C_{avg,impaired} may be above their respective therapeutic ranges.

Seltorexant or a pharmaceutically acceptable salt or hydrates thereof is preferably administered to the subject prior to sleep. For example, the compound is administered within about 2 hours of sleep, within about 1 hour, or within about 30 minutes before sleep. In other embodiments, the compound is administered at least about 4 hours before the subject wakes or intends to wake from sleep, including about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, or about 12 hours before the subject wakes or intends to wake from sleep. In certain embodiments, the compound is administered at least 6 hours to about 12 hours before the subject wakes or intends to wake from sleep. In preferred embodiments, the compound is administered at night.

After administration of the compound, the compound undergoes at least one half-life before the subject wakes from sleep. In other embodiments, the compound undergoes at least two half-lives, and preferably at least three half-lives before the subject wakes from sleep. For example, a pharmaceutical composition comprising seltorexant or a pharmaceutically acceptable salt or hydrate thereof has an elimination half-life of about 4 hours and typically less than about 4 hours. The elimination half-life may be about 2 to about 3 hours, e.g., about 2 hours, about 2.1 hours, about 2.2 hours, about 2.3 hours, about 2.4 hours, about 2.5 hours, about 2.6 hours, about 2.7 hours, about 2.8 hours, or about 2.9 hours to about 3 hours. Given the short half-life, the amount of the compound remaining in the subject upon waking is typically below the threshold required for pharmacodynamic effect.

The pharmacokinetic profile or parameter(s) (e.g., maximum plasma concentration (Cₘₐₓ), average plasma concentration (C_{avg}) and/or AUC) described herein are determined based on the subject having been fed, more specifically, based on oral administration at bedtime approximately 3 hours after the start of a standard dinner.

In some examples, the normal daily dosage amount of seltorexant or a pharmaceutically acceptable salt or hydrate thereof is selected so that a time to reach a maximum plasma concentration (Cₘₐₓ) of seltorexant is less than about 3 hours, less than about 2 hours, and preferably less than about 1 hour, i.e., less than about 45 minutes, less than about 30 minutes, less than about 15 minutes, among others, when the normal daily dosage amount is administered to a subject having normal hepatic function and/or normal ability to metabolize or eliminate seltorexant. Similarly, the lower dosage amount of seltorexant or a pharmaceutically acceptable salt or hydrate thereof is selected so that a time to reach a Cₘₐₓ of seltorexant is less than about 3 hours, less than about 2 hours, and preferably less than about 1 hour, i.e., less than about 45 minutes, less than about 30 minutes, less than about 15 minutes, among others when the lower dosage amount is administered to a subject having a hepatic impairment.

In other examples, the normal daily dosage amount of seltorexant or a pharmaceutically acceptable salt or hydrate thereof is selected so that total and/or unbound exposure (e.g., concentration in plasma) of seltorexant in a subject having normal hepatic function and/or normal ability to metabolize or eliminate seltorexant is below the therapeutic range after about 6 to about 8 hours after oral administration. Similarly, the lower dosage amount of seltorexant or a pharmaceutically acceptable salt or hydrate thereof is selected so that total and/or unbound exposure (e.g., concentration in plasma) of seltorexant in a subject having a hepatic impairment is below the therapeutic range after about 6 to about 8 hours after oral administration. Therefore, in these examples, after about 8 hours of administration of seltorexant or a pharmaceutically acceptable salt or hydrate thereof, the plasma concentration of seltorexant will fall below the therapeutic range and remain at those levels for the reminder of the 24-hour treatment period until the next daily dose is administered.

The normal daily dosage amount of seltorexant adapted for the treatment of those subjects with normal hepatic function and/or normal ability to metabolize or eliminate seltorexant is about 10 mg/day to about 60 mg/day. In some embodiments, the normal daily dosage amount is about 10 mg, about 15 mg, about 20 mg, about 25 mg, about 30 mg, about 35 mg, about 40 mg, about 45 mg, about 50 mg, about 55 mg, about 60 mg, or within a range defined by any two of these values. In a particular example, the normal daily dosage amount of seltorexant adapted for the treatment of those subjects, who do not have a hepatic impairment and/or a reduced ability to metabolize or eliminate seltorexant, is a daily seltorexant dosage amount greater than or equal to 20 mg/day, up to and including 60 mg/day. The reduced dose may be about 5 to about 20 mg/day, about 5 to about 15 mg/day, about 5 to about 10 mg/day, about 15 mg/day, about 10 mg/day or about 5 mg/day of seltorexant, depending on the indication (e.g., depression or insomnia) treated.

In another example, the daily dose is about 0.25 mg/kg per day to about 0.9 mg/kg per day, about 0.25 mg/kg per day to about 0.8 mg/kg per day, about 0.25 mg/kg per day to about 0.7 mg/kg per day, or about 0.25 mg/kg per day to about 0.6 mg/kg per day. The reduced dose is about 0.07 mg/day to about 0.3 mg/day, about 0.07 mg/day to about 0.25 mg/day, or about 0.07 mg/day to about 0.2 mg/day.

All dosage amounts mentioned herein, unless otherwise indicated, refers to the free form (i.e., free base or free base equivalent, non-salt or non-hydrate form) of seltorexant. The amounts are recited as free-form equivalents, i.e., quantities as if the free form would be administered. If salts or solvates are administered the amounts need to be calculated in function of the molecular weight ratio between the salt or solvate and the free form. The doses described herein, unless otherwise indicated, are calculated for an average body weight of about 70 kg and should be adjusted in case of pediatric applications, or when used with adult patients with a substantially diverting body weight. In some examples, the doses described herein may be adjusted for an average body weight of about 60 kg.

In some examples, seltorexant or a pharmaceutically acceptable salt or hydrate thereof is orally administered to the subject. If the subject has moderate hepatic impairment, the subject having the moderate hepatic impairment is orally administered seltorexant or a pharmaceutically acceptable salt or hydrate thereof at a lower dosage amount that is less than the normal daily dosage amount. If the subject has severe hepatic impairment, the subject is not administered seltorexant or a pharmaceutically acceptable salt or hydrate thereof (i.e., the subject is excluded from treatment with seltorexant). In some examples, oral administration of seltorexant or a pharmaceutically acceptable salt or hydrate thereof at the lower dosage amount to the subject having moderate hepatic impairment may be more therapeutically effective than the normal daily dosage amount in the subject having moderate hepatic impairment. In one example, oral administration of seltorexant or a pharmaceutically acceptable salt or hydrate thereof at the lower dosage amount to the subject having moderate hepatic impairment may be more therapeutically effective for treating depression than the normal daily dosage amount in the subject having moderate hepatic impairment.

The lower dosage amount in a subject having moderate hepatic impairment may provide a maximum plasma concentration or an average plasma concentration within their respective therapeutic ranges required for pharmacodynamic effect. A lower dosage amount may be selected so that when the lower dosage amount is administered to subjects having moderate hepatic impairment, the maximum plasma concentration (C_{max, lower}) or average plasma concentration (C_{avg,lower}) provided by the lower dosage amount in subjects who have moderate hepatic impairment is within the therapeutic ranges for Cₘₐₓ or C_{avg.}, and is therefore, more pharmacodynamically effective than C_{max,impaired} or C_{avg,impaired}. More particularly, the C_{max, lower} or C_{avg,lower} may be within their respective therapeutic ranges. Therefore, the lower dosage amount of seltorexant or a pharmaceutically acceptable salt or hydrate thereof may be more effective for treating and/or preventing depression in those subjects having moderate hepatic impairment than the normal daily dosage amount of seltorexant adapted for the treatment of those subjects who do not have a hepatic impairment and/or who do not have a reduced ability to metabolize or eliminate seltorexant. If the subject has mild hepatic impairment (e.g., a Child-Pugh score from 5 to 6), then the subject with mild hepatic impairment is administered seltorexant or a pharmaceutically acceptable salt or hydrate thereof at a normal daily dosage amount.

In a particular example, the normal daily dosage amount of seltorexant or a pharmaceutically acceptable salt or hydrate thereof is selected so that the unbound average plasma concentration (C_{avg}) of seltorexant is between about 0.5 ng/ml and about 2 ng/ml, and has been observed in a Phase 2 clinical trial to be about 0.75 ng/ml and about 1.5 ng/ml when the normal daily dosage amount is administered to a subject without a reduced ability to metabolize or eliminate seltorexant (e.g., without hepatic impairment)

The compounds described herein may be administered to a human subject for the treatment and/or prevention of an orexin-2 mediated disorder, for example, depression (e.g., MDD or bipolar depression) or insomnia, where the subject has an inadequate response to an antidepressant other than seltorexant or a pharmaceutically acceptable salt or hydrate thereof (such as, for example, selective serotonin reuptake inhibitor (SSRI) and/or serotonin-norepinephrine reuptake inhibitor (SNRI)). The compounds described herein may be administered to a human subject for the treatment and/or prevention of an orexin-2 mediated disorder, for example, depression (e.g., MDD or bipolar depression) or insomnia, where the subject has an inadequate response to a SSRI, or a SNRI. More specifically, the compounds described herein may be administered with a second pharmaceutically active agent to a human subject for the treatment and/or prevention of an orexin-2 mediated disorder, for example, depression (e.g., MDD or bipolar depression) or insomnia, where the subject has an inadequate response to a SSRI and/or a SNRI. In some examples, the second pharmaceutically active agent is a SSRI or a SNRI.

Subjects have an inadequate response to a treatment when the treatment partially reduces severity of symptoms, but the subject continues to have symptoms. More specifically, an inadequate response may be defined as less than 50% reduction but with some improvement (that is, improvement greater than 0%) in symptom severity. For example, subjects have an inadequate response to a treatment for depression when the treatment partially reduces severity of depression symptoms, but the subject continues to have depression symptoms. Specifically, an inadequate response may be defined as less than 50% reduction but with some improvement (that is, improvement greater than 0%) in depressive symptom severity with residual symptoms other than insomnia present, and overall good tolerability. The level of response to a treatment may be assessed by the Massachusetts General Hospital Antidepressant Treatment Response Questionnaire (MGH-ATRQ), which is a self-rated scale used to determine treatment resistance in MDD.

In particular examples, the present application provides methods for treating and/or preventing depression (e.g., MDD or bipolar depression) in a subject in need thereof by administering a therapeutically effective amount of seltorexant or a pharmaceutically acceptable salt or hydrate thereof to the subject when the subject is not hepatically impaired or has mild hepatic impairment and a reduced amount of seltorexant or a pharmaceutically acceptable salt or hydrate thereof to the subject when the subject has moderate hepatic impairment. The subject is a human having MDD or bipolar depression in need of preventing and/or reducing depression symptoms. In one example, the subject is a human having MDD. Specifically, the subject is a human having MDD who have had an inadequate response to conventional antidepressants. In another example, the subject is a human having bipolar depression. In one example, the subject is a human having depression, more specifically, MDD, or bipolar depression in need of preventing and/or reducing depression symptoms who have had an inadequate response to a SSRI and/or a SNRI.

Conventional antidepressant include, for example, N-methyl-D-aspartate receptor antagonists (NMDAs), norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), noradrenergic and specific serotonergic antidepressants (NaSSAs), corticotropin releasing factor (CRF) antagonists, alpha-adrenoreceptor antagonists and atypical antidepressants. In some embodiments, the N-methyl-D-aspartate (NMDA) receptor antagonist is ketamine including its racemates esketamine, arketamine, or combinations thereof. In further embodiments, the norepinephrine reuptake inhibitor includes amitriptyline, clomipramine, doxepin, imipramine, trimipramine, amoxapine, desipramine, maprotiline, nortriptyline, protriptyline, reboxetine, or pharmaceutically acceptable salts thereof. In other embodiments, the selective serotonin reuptake inhibitor (SSRI) includes fluoxetine, fluvoxamine, paroxetine, sertraline, or pharmaceutically acceptable salts thereof. In further embodiments, the monoamine oxidase inhibitor (MAOI) includes isocarboxazid, phenelzine, tranylcypromine, selegiline and pharmaceutically acceptable salts thereof. In yet other embodiments, the reversible inhibitor of monoamine oxidase (RIMA) includes moclobemide or pharmaceutically acceptable salts thereof. In still further embodiments, the serotonin and noradrenaline reuptake inhibitor (SNRI) includes citalopram, duloxetine, escitalopram, fluvoxamine, fluoxetine, milnacipran, levomilnacipran, paroxetine, sertraline, venlafaxine, desvenlafaxine, vilazodone, vortioxetine, or pharmaceutically acceptable salts thereof. In other embodiments, the atypical antidepressant includes bupropion, lithium, nefazodone, trazodone, viloxazine, sibutramine, or pharmaceutically acceptable salts thereof.

In yet further embodiments, the conventional antidepressant includes adinazolam, alaproclate, amineptine, amitriptyline/chlordiazepoxide combination, atipamezole, azamianserin, bazinaprine, befuraline, bifemelane, binodaline, bipenamol, brofaromine, bupropion, caroxazone, cericlamine, cianopramine, cimoxatone, citalopram, clemeprol, clovoxamine, dazepinil, deanol, demexiptiline, dibenzepin, dothiepin, droxidopa, enefexine, estazolam, etoperidone, femoxetine, fengabine, fezolamine, fluotracen, idazoxan, indalpine, indeloxazine, iprindole, levoprotiline, litoxetine, lofepramine, medifoxamine, metapramine, metralindole, mianserin, milnacipran, minaprine, mirtazapine, monirclin, nebracetam, nefopam, nialamide, nomifensine, norfluoxetine, orotirelin, oxaflozane, pinazepam, pirlindone, pizotyline, ritanserin, rolipram, sercloremine, setiptiline, sibutramine, sulbutiamine, sulpiride, teniloxazine, thozalinone, thymoliberin, tianeptine, tiflucarbine, tofenacin, tofisopam, toloxatone, tomoxetine, veralipride, viqualine, zimelidine zometapine, or pharmaceutically acceptable salts thereof; or St. John's wort herb, Hypericum perforatum, or extracts thereof.

The present application includes methods comprising administering seltorexant or a pharmaceutically acceptable salt or hydrate thereof to prevent and/or reduce severity of depression or at least one symptom of depression in a subject when the subject is not hepatically impaired or has mild hepatic impairment and a reduced amount of seltorexant or a pharmaceutically acceptable salt or hydrate thereof to the subject when the subject has moderate hepatic impairment. In one example, the method comprises administering seltorexant or a pharmaceutically acceptable salt or hydrate thereof to prevent and/or reduce a measurement quantifying a collective severity of a plurality of depression symptoms (e.g., a set of 3, 4, 5, 6, 7, 8, 9, 10, 12 or 15 depression symptoms) in a patient having depression, specifically, MDD, or bipolar depression. The symptoms of depression reduced by administration of seltorexant or a pharmaceutically acceptable salt or hydrate thereof may include, for example, apparent sadness, reported sadness, inner tension, reduced sleep, reduced appetite, concentration difficulties, lassitude, inability to feel, and/or pessimistic thoughts.

The prevention and/or reduction of severity of depression may be observed qualitatively (e.g., by general patient evaluation by a clinician during visits to a clinic) or as a quantitative reduction in scores over a period of time (e.g., 1 week, 2 weeks, 4 weeks, 6 weeks, 8 weeks, 12 weeks, etc.) determined using any suitable clinical scale (e.g., diagnostic questionnaires) for measuring severity of depression symptoms as would be understood by those of ordinary skill in the art, such as, for example, Clinical Global Impression-Severity (CGI-S) scale, EuroQol; 5 dimension; 5 level (EQ-5D-5L), Patient Health Questionnaire-9 Item (PHQ-9), Shechan Disability Scale (SDS), Inventory of Depressive Symptomatology-Clinician rated, 30-item scale (IDS-C₃₀), Montgomery-Asberg Depression Rating Scale (MADRS), Hamilton rating scale for depression (HAM-D or HDRS) Beck Scale for Depression, Quick Inventory of Depressive Symptomology (QIDS), 17-item Hamilton Depression Rating Scale (HDRS₁₇), Patient Health Questionnaire (PHQ-9), Insomnia Severity Index (ISI) and/or PROMIS-Sleep Disturbance (PROMIS-SD).

In a particular example, the method comprises administering seltorexant or a pharmaceutically acceptable salt or hydrate thereof to prevent and/or reduce a measurement quantifying a collective severity of 10 depression symptoms (e.g., Montgomery-Asberg Depression Rating Scale (MADRS) score) in a subject having depression, specifically, MDD, or bipolar depression, when the subject is not hepatically impaired or has mild hepatic impairment and a reduced amount of seltorexant or a pharmaceutically acceptable salt or hydrate thereof to the subject when the subject has moderate hepatic impairment. The MADRS is a clinician-rated scale designed to measure depression severity and detects changes due to antidepressant treatment. The scale consists of 10 items, each of which is scored from 0 (Item not present or normal) to 6 (severe or continuous presence of the symptoms), for a total possible score of 60. Higher scores represent a more severe condition. The MADRS evaluates apparent sadness, reported sadness, inner tension, reduced sleep, reduced appetite, concentration difficulties, lassitude, inability to feel, pessimistic thoughts, and suicidal thoughts.

Seltorexant or a pharmaceutically acceptable salt or hydrate thereof may be administered as a monotherapy or may be administer in combination with or adjunctively with other treatments, such as a second pharmaceutically active agent. By administered adjunctively, it is meant that seltorexant or a pharmaceutically acceptable salt or hydrate thereof is used together with another treatment to assist the other treatment, for example, seltorexant or a pharmaceutically acceptable salt or hydrate thereof is administered while the patient is undergoing the other treatment. Seltorexant or a pharmaceutically acceptable salt or hydrate thereof in combination or adjunctively with another pharmaceutically active agent may serve to increase efficacy (e.g., by potentiating the potency or effectiveness of the other pharmaceutically active agent), decrease one or more side effects, or decrease the required dose of seltorexant or a pharmaceutically acceptable salt or the other pharmaceutically active agent.

The other treatment may be administration of another pharmaceutically active agent or psychotherapy (e.g., interpersonal and social rhythm therapy (IPSRT) or cognitive behavior therapy (CBT)). The other pharmaceutically active agent may be any one or more of the conventional antidepressants identified above. In one example, seltorexant or a pharmaceutically acceptable salt hydrate thereof and another pharmaceutically active agent may be administered over the same period of time. More specifically, the subject may be undergoing therapy where both seltorexant or a pharmaceutically acceptable salt or hydrate thereof and the other pharmaceutically active agent are administered within the same day (e.g., a 24-hour period). Seltorexant or a pharmaceutically acceptable salt or hydrate thereof and the other pharmaceutically active agent may be simultaneously administered, administered at separate times, or administered intermittently. The length of time between administration of the two therapeutic agents may be adjusted to achieve the desired therapeutic effect. For example, one therapeutic agent may be administered only a few minutes (e.g., about 1, 2, 5, 10, 30, or 60 minutes) after administration of the other therapeutic agent. Alternatively, one therapeutic agent may be administered several hours (e.g., about 2, 4, 6, 10, 12, 24, or 36 hours) after administration of the other therapeutic agent. In certain embodiments, it may be advantageous to administer more than one dosage of one agent between administrations of the other therapeutic agent. In certain embodiments of the present invention, it may be that the therapeutic effects of the two therapeutic agents overlap for at least a portion of the duration of each therapeutic agent.

In some embodiments, seltorexant or a pharmaceutically acceptable salt or hydrate thereof is adjunctive administered with a conventional antidepressant. For example, seltorexant or a pharmaceutically acceptable salt or hydrate thereof is adjunctively administered with a SSRI or a SNRI. In other embodiments, seltorexant or a pharmaceutically acceptable salt or hydrate thereof is co-administered with esketamine. In further embodiments, seltorexant or a pharmaceutically acceptable salt or hydrate thereof is administered separately from esketamine such as, e.g., sequentially. Seltorexant or a pharmaceutically acceptable salt or hydrate thereof may be administered prior or subsequent to esketamine.

Seltorexant or a pharmaceutically acceptable salt or hydrate thereof may be administered by any suitable administration route, such as, for example, orally, parenterally (including subcutaneously (s.c.), intramuscularly (i.m.), and intravenously (i.v.)), rectally, transdermally, buccally, or nasally. The compounds may also be administered directly to the nervous system by routes including, but not limited to, intracerebral, intraventricular, intracerebroventricular, intrathecal, intracisternal, intraspinal and/or peri-spinal route by delivery via intracranial or intravertebral needles and/or catheters with or without pump devices. Suitable compositions for oral administration include powders, granulates, aggregates, tablets, compressed or coated pills, hard or gelatin capsules, syrups, emulsions and suspensions. Suitable compositions for parenteral administration include aqueous or non-aqueous solutions or emulsions. In a preferred embodiment, the compounds are administered orally, such as, for example, by a tablet, a gelatin capsule or an orally ingested liquid.

The compounds described herein may be formulated as a pharmaceutical composition for administration to a human subject. Accordingly, a pharmaceutical composition may comprise (a) an effective amount of the compound described herein and (b) a pharmaceutically acceptable excipient. A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the compounds described herein may be prepared using suitable pharmaceutical excipients and compounding techniques known or that become available to those skilled in the art. The preparation may be in the form of tablets, capsules, sachets, dragees, powders, granules, lozenges, powders for reconstitution, or liquid preparations. In some embodiments, the compositions are formulated for intravenous infusion, topical administration, or oral administration. In certain embodiments, the compositions are formulated for immediate release.

For oral administration, the compounds can be provided in the form of tablets or capsules, or as a solution, emulsion, or suspension. In certain embodiments, the compounds may be taken with food.

Oral tablets may include a compound described herein mixed with pharmaceutically acceptable excipients such as inert fillers, diluents, disintegrating agents, binding agents, lubricating agents, sweetening agents, flavoring agents, coloring agents, glidants and preservative agents. Suitable inert fillers include sodium and calcium carbonate, sodium and calcium phosphate, lactose, lactose monohydrate, starch, sugar, glucose, methyl cellulose, magnesium stearate, mannitol, sorbitol, hypromellose, and the like. Exemplary liquid oral excipients include ethanol, glycerol, water, and the like. Starch, polyvinyl-pyrrolidone, sodium starch glycolate, microcrystalline cellulose, crospovidone (cross-linked polyvinyl N-pyrrolidone or PVP), and alginic acid are suitable disintegrating agents. Binding agents may include hypromellose (hydroxypropyl methylcellulose or HPMC), starch and gelatin. The lubricating agent, if present, may be magnesium stearate, stearic acid, or talc. The glidant, if present, may be silica (Sift) such as colloidal silica. If desired, the tablets may be coated with a material such as glyceryl monostearate or glyceryl distearate to delay absorption in the gastrointestinal tract, or may be coated with an enteric coating.

In an exemplary embodiment, a tablet containing seltorexant includes the components set forth in Table 1 below.

TABLE 1

| | Quantity/Unit Dose | |
|---|---|---|
| Component | (in mg/tablet) | (in % w/w) |
| Seltorexant | 20 | 13.3 |
| Lactose monohydrate | 80 | 53.3 |
| Microcrystalline cellulose | 40.5 | 27 |
| Crospovidone | 7.5 | 5 |
| Silica colloidal | 1 | 0.7 |
| Magnesium stearate | 1 | 0.7 |
| Total | 150 | 100 |

Capsules for oral administration include hard and soft gelatin capsules. To prepare hard gelatin capsules, the compound may be mixed with a solid, semi-solid, or liquid diluent. Soft gelatin capsules may be prepared by mixing the compound with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups or may be lyophilized or presented as a dry product for reconstitution with water or other suitable vehicle before use. Such liquid compositions may optionally contain pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The compounds described herein may also be formulated for administration by non-oral routes. For example, the compounds may be formulated for rectal administration. For parenteral use, including intravenous, intramuscular, or intraperitoneal routes, the compound may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of the compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compound may utilize a patch formulation to affect transdermal delivery.

Compounds may alternatively be formulated for administered by inhalation, via the nasal or oral routes, e.g., in a spray formulation also containing a suitable carrier.

### Aspects

Aspect 1. A method of treating an orexin-2 mediated disorder in a human subject in need thereof, the method comprising:
administering seltorexant or a pharmaceutically acceptable salt or hydrate thereof at a therapeutically effective daily dose to the subject, when the subject does not have a hepatic impairment; and
administering seltorexant or the pharmaceutically acceptable salt or hydrate thereof at a reduced dose less than the daily dose to the subject, when the subject has moderate hepatic impairment.

Aspect 2. The method of Aspect 1, further comprising:
administering seltorexant or a pharmaceutically acceptable salt or hydrate thereof at the daily dose to the subject, when the subject has mild hepatic impairment;

Aspect 3. The method of Aspect 1, further comprising:
not administering seltorexant or a pharmaceutically acceptable salt or hydrate to the subject, when the subject has severe hepatic impairment.

Aspect 4. The method of Aspect 2, further comprising:
not administering seltorexant or a pharmaceutically acceptable salt or hydrate to the subject, when the subject has severe hepatic impairment.

Aspect 5. The method of Aspect 2, wherein the subject having the mild hepatic impairment has a Child-Pugh score from 5 to 6.

Aspect 6. The method of any one of Aspects 1 to 4, wherein the subject having the moderate hepatic impairment has a Child-Pugh score from 7 to 9.

Aspect 7. The method of Aspect 3 or 4, wherein the subject having the severe hepatic impairment has a Child-Pugh score at or above 10.

Aspect 8. The method of any one of Aspects 1 to 4, wherein seltorexant is administered as a free base.

Aspect 9. The method of any one of Aspects 1 to 4, wherein a hydrochloride salt of seltorexant is administered.

Aspect 10. The method of any one of Aspects 1 to 4, wherein the orexin-2 mediated disorder is selected from the group consisting of depression and insomnia.

Aspect 11. The method of Aspect 10, wherein the orexin-2 mediated disorder is depression.

Aspect 12. The method of Aspect 11, wherein the depression is major depressive disorder.

Aspect 13. The method of Aspect 11, wherein the depression is bipolar depression.

Aspect 14. The method of Aspect 11, wherein the subject has concurrent depression and insomnia symptoms having an ISI score of greater than or equal to 15.

Aspect 15. The method of Aspect 10, wherein the orexin-2 mediated disorder is insomnia.

Aspect 16. The method of any one of Aspects 1 to 4, wherein seltorexant is orally administered.

Aspect 17. The method of any one of Aspects 1 to 4, wherein the daily dose is about 20 mg/day to about 60 mg/day.

Aspect 18. The method of Aspect 17, wherein the reduced dose is about 5 mg/day to about 20 mg/day.

Aspect 19. The method of Aspect 18, wherein the reduced dose is about 20 mg/day.

Aspect 20. The method of Aspect 18, wherein the reduced dose is about 15 mg/day.

Aspect 21. The method of Aspect 18, wherein the reduced dose is about 10 mg/day.

Aspect 22. The method of Aspect 18, wherein the reduced dose is about 5 mg/day.

Aspect 23. The method of any one of Aspects 1 to 4, wherein the reduced dose is at least 20% less than the daily dose.

Aspect 24. The method of any one of Aspects 1 to 4, wherein the reduced dose is at least 25% less than the daily dose.

Aspect 25. The method of any one of Aspects 1 to 4, wherein the reduced dose is about 50% less than the daily dose.

Aspect 26. The method of any one of Aspects 1 to 4, wherein the daily dose is about 0.25 mg/kg per day to about 0.9 mg/kg per day, and the reduced dose is about 0.07 mg/day to about 0.3 mg/day.

Aspect 27. The method of any one of Aspects 1 to 4, wherein administration of seltorexant or the pharmaceutically acceptable salt or hydrate thereof at the daily dose to the subject provides an unbound average plasma concentration of seltorexant between about 0.75 ng/ml and about 1.5 ng/mL, when the subject does not have a hepatic impairment.

Aspect 28. The method of any one of Aspects 11 to 14, wherein
administration of seltorexant or the pharmaceutically acceptable salt or hydrate thereof at the daily dose to the subject provides a first unbound average plasma concentration of seltorexant within an effective therapeutic range, when the subject does not have a hepatic impairment; and
administration of seltorexant or the pharmaceutically acceptable salt of hydrate thereof at the reduced dose to the subject provides a second unbound average plasma concentration of seltorexant within the effective therapeutic range, when the subject has moderate hepatic impairment.

Aspect 29. The method of Aspect 28, wherein administration of seltorexant or the pharmaceutically acceptable salt or hydrate thereof at the daily dose to the subject provides a third unbound average plasma concentration of seltorexant within the effective therapeutic range, when the subject has mild hepatic impairment.

Aspect 30. The method of any one of Aspects 11 to 14, wherein administration of seltorexant or the pharmaceutically acceptable salt or thereof at the reduced dose is more therapeutically effective than the daily dose in the subject, when the subject has the moderate hepatic impairment.

Aspect 31. The method of any one of Aspects 11 to 14, wherein the subject has an inadequate response to an antidepressants other than seltorexant or a pharmaceutically acceptable salt or hydrate thereof.

Aspect 32. The method of Aspect 31, wherein the subject has an inadequate response to a selective serotonin reuptake inhibitor (SSRI) or a serotonin and noradrenaline reuptake inhibitor (SNRI).

Aspect 33. The method of any one of Aspects 1 to 4, wherein seltorexant or a pharmaceutically acceptable salt or hydrate thereof is adjunctively administered with a second pharmaceutically active agent.

Aspect 34. The method of Aspect 33, wherein the second pharmaceutically active agent is a selective serotonin reuptake inhibitor (SSRI) or a serotonin and noradrenaline reuptake inhibitor (SNRI).

Aspect 35. A method of treating an orexin-2 mediated disorder in a human subject in need thereof, the subject having moderate hepatic impairment, the method comprising:

administering seltorexant or a pharmaceutically acceptable salt or hydrate thereof at a reduced dose that is less than a therapeutically effective daily dose adapted for treatment of a human subject having normal hepatic function.

Aspect 36. The method of Aspect 35, wherein the subject having the moderate hepatic impairment has a Child-Pugh score from 7 to 9.

Aspect 37. The method of Aspect 35, wherein seltorexant is administered as a free base.

Aspect 38. The method of Aspect 35, wherein a hydrochloride salt of seltorexant is administered.

Aspect 39. The method of Aspect 35, wherein the orexin-2 mediated disorder is selected from the group consisting of depression and insomnia.

Aspect 40. The method of Aspect 39, wherein the orexin-2 mediated disorder is depression.

Aspect 41. The method of Aspect 39, wherein the depression is major depressive disorder.

Aspect 42. The method of Aspect 39, wherein the depression is bipolar depression.

Aspect 43. The method of Aspect 39, wherein the subject has concurrent depression and insomnia symptoms having an ISI score of greater than or equal to 15.

Aspect 44. The method of Aspect 39, wherein the orexin-2 mediated disorder is insomnia.

Aspect 45. The method of any one of Aspects 35 to 44, wherein seltorexant is orally administered.

Aspect 46. The method of any one of Aspect 35 to 44, wherein the daily dose is about 20 mg/day to about 60 mg/day.

Aspect 47. The method of Aspect 46, wherein the reduced dose is about 5 mg/day to about 20 mg/day.

Aspect 48. The method of Aspect 47, wherein the reduced dose is about 20 mg/day.

Aspect 49. The method of Aspect 47, wherein the reduced dose is about 15 mg/day.

Aspect 50. The method of Aspect 47, wherein the reduced dose is about 10 mg/day.

Aspect 51. The method of Aspect 47, wherein the reduced dose is about 5 mg/day.

Aspect 52. The method of any one of Aspects 35 to 45, wherein the reduced dose is at least 20% less than the daily dose.

Aspect 53. The method any one of Aspects 35 to 45, wherein the reduced dose is at least 25% less than the daily dose.

Aspect 54. The method of any one of Aspects 35 to 45, wherein the reduced dose is about 50% less than the daily dose.

Aspect 55. The method of any one of Aspects 35 to 45, wherein the daily dose is about 0.25 mg/kg per day to about 0.9 mg/kg per day, and the reduced dose is about 0.07 mg/day to about 0.3 mg/day.

Aspect 56. The method of any one of Aspects 35 to 45, wherein the daily dose is selected to provide an unbound average plasma concentration of seltorexant between about 0.75 ng/ml and about 1.5 ng/mL when administered the human subject having normal hepatic function.

Aspect 57. The method of any one of Aspects 40 to 43, wherein administration of seltorexant or the pharmaceutically acceptable salt of hydrate thereof at the reduced dose to the subject having moderate hepatic impairment provides an unbound average plasma concentration of seltorexant within an effective therapeutic range.

Aspect 58. The method of any one of Aspects 40 to 43, wherein administration of seltorexant or the pharmaceutically acceptable salt or thereof at the reduced dose is more therapeutically effective than the daily dose in the subject having moderate hepatic impairment.

Aspect 59. The method of any one of Aspects 40 to 43, wherein the subject has an inadequate response to an antidepressants other than seltorexant or a pharmaceutically acceptable salt or hydrate thereof.

Aspect 60. The method of Aspect 59, wherein the subject has an inadequate response to a selective serotonin reuptake inhibitor (SSRI) or a serotonin and noradrenaline reuptake inhibitor (SNRI).

Aspect 61. The method of any one of Aspects 35 to 45, wherein seltorexant or a pharmaceutically acceptable salt or hydrate thereof is adjunctively administered with a second pharmaceutically active agent.

Aspect 62. The method of Aspect 61, wherein the second pharmaceutically active agent is a selective serotonin reuptake inhibitor (SSRI) or a serotonin and noradrenaline reuptake inhibitor (SNRI).

Aspect 63. A method of treating an orexin-2 mediated disorder in a human subject in need thereof, the subject having mild hepatic impairment, the method comprising:

administering seltorexant or a pharmaceutically acceptable salt or hydrate thereof at a therapeutically effective daily dosage amount adapted for treatment of a human subject having normal hepatic function.

Aspect 64. The method of Aspect 63, wherein the subject having the mild hepatic impairment has a Child-Pugh score from 5 to 6.

Aspect 65. The method of Aspect 63, wherein seltorexant is administered as a free base.

Aspect 66. The method of Aspect 63, wherein a hydrochloride salt of seltorexant is administered.

Aspect 67. The method of Aspect 63, wherein the orexin-2 mediated disorder is selected from the group consisting of depression and insomnia.

Aspect 68. The method of Aspect 67, wherein the orexin-2 mediated disorder is depression.

Aspect 69. The method of Aspect 68, wherein the depression is major depressive disorder.

Aspect 70. The method of Aspect 68, wherein the depression is bipolar depression.

Aspect 71. The method of Aspect 67, wherein the subject has concurrent depression and insomnia symptoms having an ISI score of greater than or equal to 15.

Aspect 72. The method of Aspect 67, wherein the orexin-2 mediated disorder is insomnia.

Aspect 73. The method of any one of Aspects 63 to 72, wherein seltorexant is orally administered.

Aspect 74. The method of any one of Aspects 63 to 72, wherein the daily dose is about 20 mg/day to about 60 mg/day.

Aspect 75. The method of any one of Aspects 63 to 72, wherein the daily dose is about 0.25 mg/kg per day to about 0.9 mg/kg per day.

Aspect 76. The method of Aspect 63, wherein the daily dose is selected to provide an unbound average plasma concentration of seltorexant between about 0.75 ng/ml and about 1.5 ng/mL when administered the human subject having normal hepatic function.

Aspect 77. The method of any one of Aspects 68 to 71, wherein administration of seltorexant or the pharmaceutically acceptable salt or hydrate thereof at the daily dose to the subject having mild hepatic impairment provides an unbound average plasma concentration of seltorexant within an effective therapeutic range.

Aspect 78. The method of any one of Aspects 68 to 71, wherein the subject has an inadequate response to an antidepressants other than seltorexant or a pharmaceutically acceptable salt or hydrate thereof.

Aspect 79. The method of Aspect 78, wherein the subject has an inadequate response to a selective serotonin reuptake inhibitor (SSRI) or a serotonin and noradrenaline reuptake inhibitor (SNRI).

Aspect 80. The method of any one of Aspects 68 to 71, wherein seltorexant or a pharmaceutically acceptable salt or hydrate thereof is adjunctively administered with a second pharmaceutically active agent.

Aspect 81. The method of Aspect 80, wherein the second pharmaceutically active agent is a selective serotonin reuptake inhibitor (SSRI) or a serotonin and noradrenaline reuptake inhibitor (SNRI).

Aspect 82. A method of treating an orexin-2 mediated disorder in a human subject in need thereof, the method comprising:
if the subject has moderate hepatic impairment, administering seltorexant or a pharmaceutically acceptable salt or hydrate thereof at a reduced dose that is less than a therapeutically effective daily dose adapted for treatment of a human subject having normal hepatic function;
if the subject has severe hepatic impairment, not administering seltorexant or a pharmaceutically acceptable salt or hydrate to the subject; and
if the subject does not have moderate to severe hepatic impairment, administering seltorexant or a pharmaceutically acceptable salt or hydrate thereof at the daily dose to the subject.

Aspect 83. The method of Aspect 82, wherein the subject having the mild hepatic impairment has a Child-Pugh score from 5 to 6.

Aspect 84. The method of Aspect 82, wherein the subject having the moderate hepatic impairment has a Child-Pugh score from 7 to 9.

Aspect 85. The method of Aspect 82, wherein the subject having the severe hepatic impairment has a Child-Pugh score at or above 10.

Aspect 86. The method of Aspect 82, wherein seltorexant is administered as a free base.

Aspect 87. The method of Aspect 82, wherein a hydrochloride salt of seltorexant is administered.

Aspect 88. The method of Aspect 82, wherein the orexin-2 mediated disorder is selected from the group consisting of depression and insomnia.

Aspect 89. The method of Aspect 88, wherein the orexin-2 mediated disorder is depression.

Aspect 90. The method of Aspect 89, wherein the depression is major depressive disorder.

Aspect 91. The method of Aspect 89, wherein the depression is bipolar depression.

Aspect 92. The method of Aspect 89, wherein the subject has concurrent depression and insomnia symptoms having an ISI score of greater than or equal to 15.

Aspect 93. The method of Aspect 88, wherein the orexin-2 mediated disorder is insomnia.

Aspect 94. The method of any one of Aspects 80 to 93, wherein seltorexant is orally administered.

Aspect 95. The method of any one of Aspects 80 to 93, wherein the daily dose is about 20 mg/day to about 60 mg/day.

Aspect 96. The method of Aspect 95, wherein the reduced dose is about 5 mg/day to about 20 mg/day.

Aspect 97. The method of Aspect 95, wherein the reduced dose is about 20 mg/day.

Aspect 98. The method of Aspect 95, wherein the reduced dose is about 15 mg/day.

Aspect 99. The method of Aspect 95, wherein the reduced dose is about 10 mg/day.

Aspect 100. The method of Aspect 95, wherein the reduced dose is about 5 mg/day.

Aspect 101. The method of any one of Aspects 80 to 93, wherein the reduced dose is at least 20% less than the daily dose.

Aspect 102. The method of any one of Aspects 80 to 93, wherein the reduced dose is at least 25% less than the daily dose.

Aspect 103. The method of any one of Aspects 80 to 93, wherein the reduced dose is about 50% less than the daily dose.

Aspect 104. The method of any one of Aspects 80 to 93, wherein the daily dose is about 0.25 mg/kg per day to about 0.9 mg/kg per day, and the reduced dose is about 0.07 mg/day to about 0.3 mg/day.

Aspect 105. The method of any one of Aspects 80 to 93, wherein the daily dose is selected to provide an unbound average plasma concentration of seltorexant between about 0.75 ng/mL and about 1.5 ng/ml when administered the human subject having normal hepatic function.

Aspect 106. The method of any one of Aspects 87 to 90, wherein administration of seltorexant or the pharmaceutically acceptable salt or thereof at the reduced dose is more therapeutically effective than the daily dose in the subject, when the subject has the moderate hepatic impairment.

Aspect 107. The method of any one of Aspects 87 to 90, wherein the subject has an inadequate response to an antidepressants other than seltorexant or a pharmaceutically acceptable salt or hydrate thereof.

Aspect 108. The method of Aspect 107, wherein the subject has an inadequate response to a selective serotonin reuptake inhibitor (SSRI) or a serotonin and noradrenaline reuptake inhibitor (SNRI).

Aspect 109. The method of any one of Aspects 80 to 93, wherein seltorexant or a pharmaceutically acceptable salt or hydrate thereof is adjunctively administered with a second pharmaceutically active agent.

Aspect 110. The method of Aspect 109, wherein the second pharmaceutically active agent is a selective serotonin reuptake inhibitor (SSRI) or a serotonin and noradrenaline reuptake inhibitor (SNRI).

### Aspects II

Aspect II-1. A method of treating an orexin-2 mediated disorder in a human subject in need thereof, the subject having moderate hepatic impairment, the method comprising:
administering seltorexant or a pharmaceutically acceptable salt or hydrate thereof at a reduced dose that is less than a therapeutically effective daily dose adapted for treatment of a human subject having normal hepatic function.

Aspect II-2. A method of treating an orexin-2 mediated disorder in a human subject in need thereof, the method comprising:
if the subject has moderate hepatic impairment, administering seltorexant or a pharmaceutically acceptable salt or hydrate thereof at a reduced dose that is less than a therapeutically effective daily dose adapted for treatment of a human subject having normal hepatic function;
if the subject has severe hepatic impairment, not administering seltorexant or a pharmaceutically acceptable salt or hydrate thereof to the subject; and
if the subject does not have moderate to severe hepatic impairment, administering seltorexant or a pharmaceutically acceptable salt or hydrate thereof at the daily dose to the subject.

Aspect II-3. The method of Aspect II-1 or Aspect II-2, wherein the subject having the moderate hepatic impairment has a Child-Pugh score from 7 to 9.

Aspect II-4. The method of any one of Aspects II-1 to II-3, wherein seltorexant is administered as a free base.

Aspect II-5. The method of any one of Aspects II-1 to II-3, wherein a hydrochloride salt of seltorexant is administered.

Aspect II-6. The method of any one of Aspects II-1 to II-5, wherein the orexin-2 mediated disorder is depression or insomnia.

Aspect II-7. The method of Aspect II-6, wherein the orexin-2 mediated disorder is depression.

Aspect II-8. The method of Aspect II-7, wherein the depression is major depressive disorder.

Aspect II-9. The method of Aspect II-7, wherein the depression is bipolar depression.

Aspect II-10. The method of any one of Aspects II-7 to II-9, wherein the subject has concurrent insomnia symptoms.

Aspect II-11. The method of Aspect II-10, wherein the subject has an ISI score of greater than or equal to 15.

Aspect II-12. The method of Aspect II-6, wherein the orexin-2 mediated disorder is insomnia.

Aspect II-13. The method of any one of Aspects II-1 to II-12, wherein seltorexant is orally administered.

Aspect II-14. The method of Aspect II-13, wherein the daily dose is about 20 mg/day to about 60 mg/day.

Aspect II-15. The method of Aspect II-14, wherein the reduced dose is about 5 mg/day to about 20 mg/day.

Aspect II-16. The method of Aspect II-15, wherein the reduced dose is about 20 mg/day.

Aspect II-17. The method of Aspect II-15, wherein the reduced dose is about 15 mg/day.

Aspect II-18. The method of Aspect II-15, wherein the reduced dose is about 10 mg/day.

Aspect II-19. The method of Aspect II-15, wherein the reduced dose is about 5 mg/day.

Aspect II-20. The method of Aspect II-14, wherein the daily dose is selected to provide an unbound average plasma concentration of seltorexant between about 0.75 ng/mL and about 1.5 ng/mL when administered the human subject having normal hepatic function.

Aspect II-21. The method of Aspect II-20, wherein administration of seltorexant or the pharmaceutically acceptable salt or hydrate thereof at the reduced dose to the subject having moderate hepatic impairment provides an unbound average plasma concentration of seltorexant within an effective therapeutic range.

Aspect II-22. The method of any one of Aspects II-1 to II-21, wherein the subject has an inadequate response to an antidepressants other than seltorexant or a pharmaceutically acceptable salt or hydrate thereof.

Aspect II-23. The method of Aspect II-22, wherein the subject has an inadequate response to a selective serotonin reuptake inhibitor (SSRI) or a serotonin and noradrenaline reuptake inhibitor (SNRI).

Aspect II-24. The method of any one of Aspects II-1 to II-23, wherein seltorexant or a pharmaceutically acceptable salt or hydrate thereof is adjunctively administered with a second pharmaceutically active agent.

Aspect II-25. The method of Aspect II-24, wherein the second pharmaceutically active agent is a selective serotonin reuptake inhibitor (SSRI) or a serotonin and noradrenaline reuptake inhibitor (SNRI).

Aspect II-26. The method of Aspect II-2, wherein the subject having the mild hepatic impairment has a Child-Pugh score from 5 to 6.

Aspect II-27. The method of Aspect II-2, wherein the subject having the severe hepatic impairment has a Child-Pugh score at or above 10.

Aspect II-28. The method of any one of Aspects II-1 to II-3 or II-6 to II-27, wherein a hydrate of a hydrochloride salt of seltorexant is administered.

### EXAMPLE

The following example is to further illustrate the nature of the invention. It should be understood that the following example does not limit the invention and that the scope of the invention is to be determined by the appended claims.

### Example 1

Example 1 was an open-label, parallel-group single-dose Phase 1 clinical study to characterize the pharmacokinetics (PK) and safety of seltorexant and its metabolites, M12 and M16 in participants, between 18 and 79 years of age, inclusive, with varying stages of hepatic impairment and healthy participants (Clinical Trial Identifier: NCT04960124, a description of which is available at https://clinicaltrials.gov/ct2/show/NCT04960124 and incorporated by reference herein). This example was performed to evaluate the pharmacokinetics of a single oral dose of seltorexant in adult participants with hepatic impairment when compared to healthy participants with normal hepatic function.

Participants were screened for eligibility for study entry. The diagnosis of hepatic impairment was based on medical history, a Child-Pugh Clinical Assessment Score of 5 to 6 (Grade A, mild hepatic impairment), 7 to 9 (Grade B, moderate hepatic impairment), and 10 to 15 (Grade C, severe hepatic impairment), and the presence of physical signs (liver firmness to palpitation, splenic enlargement, spider angioma, palmar erythema, parotid hypertrophy, testicular atrophy, or gynecomastia) or other modalities (computed tomography, ultrasound, magnetic resonance imaging, radionuclide liver/spleen scan or abdominal laparoscopy, or liver biopsy). The underlying disease leading to hepatic impairment was documented (e.g., hepatitis B, hepatitis C, alcoholic steatohepatitis, nonalcoholic fatty liver disease [hereditary/metabolic], or other). Hepatic encephalopathy grading was done by the Principal Investigator. The individual components of the Child-Pugh Clinical Assessment Score were documented. Participants with liver transplant were excluded.

The clinical study of Example 1 consisted of a screening phase (within 21 days before study intervention administration); an open-label treatment phase (Day 1 to Day 5) including the end-of-study (EOS) or withdrawal assessments (performed on Day 5 or upon early withdrawal). The study duration for each participant was approximately 4 weeks from screening (Day-21 to Day-1) to EOS assessments.

Healthy men and women between 18 and 79 years of age, inclusive; with a body mass index (BMI) between 18 and 38 kg/m², inclusive, and a body weight of not less than 50 kg were enrolled in the study according to their hepatic function. The degree of hepatic impairment was based on Child Pugh's classification. Using this classification, participants were grouped on the basis of 2 clinical features (hepatic encephalopathy and ascites) and 3 laboratory-based parameters (albumin, bilirubin, and prothrombin time).

The study included 24 human patients divided into 3 different cohorts:
Cohort 1:8 healthy participants with normal hepatic function and no evidence of liver damage and no evidence of liver damage,
Cohort 2:8 participants with mild hepatic impairment (having a Child-Pugh score of 5 to 6), and
Cohort 3:8 participants with moderate hepatic impairment (Child-Pugh score of 7 to 9).

Participants in Cohort 1 and Cohort 2 were administered a single 20 mg dose of seltorexant. Participants in Cohort 3 were administered a lower dose of 10 mg dose of seltorexant since preliminary physiological based pharmacokinetic modeling and simulation (PBPK) predicted a 2.5-fold increase in total exposure for seltorexant in participants with moderate hepatic impairment.

Based on hepatic impairment classified during screening, 8 participants with mild hepatic impairment (Cohort 2) and 8 participants with moderate hepatic impairment (Cohort 3) were enrolled. After 8 participants each in Cohort 2 and Cohort 3 had completed study related procedures including the 96 hours PK sample, participants with normal hepatic function (Cohort 1) were enrolled. A group matching procedure was used to demographically match healthy participants having normal hepatic function with respect to sex, age, and weight to the participants enrolled in Cohort 2 and Cohort 3. At the time of initiation of the control group, a total of 8 participants with normal hepatic function (Cohort 1) who were of similar mean age (±10 years) and mean body weight (±10 kg) were enrolled in a similar male to female participant ratio of all combined completers from the mild (Cohort 2) and moderate (Cohort 3) hepatic impairment groups. Participants in the control group (Cohort 1) who tested positive for Hepatitis B surface antigen (HbsAg) or Hepatitis C antibodies were not permitted in the study.

In the normal hepatic function group (Cohort 1), at least 3 participants with an individual value above and at least 3 participants with an individual value below the mean age/mean body weight values of the combined mild (Cohort 2) and moderate (Cohort 3) hepatic impairment groups were enrolled. Two to 4 females were enrolled in each of the cohorts.

Of the 8 participants enrolled in Cohort 1, 4 (50.0%) were men and 4 (50.0%) were women. The mean age was 63.6 years (±6.55 years) and the mean weight was 75.10 kg (±8.76 kg). The median BMI was 25.10 kg/m² (range: 22.5 to 28.5 kg/m²). All participants were White by race and not Hispanic or Latino in ethnicity. The mean estimated glomerular filtration rate (eGFR) for this cohort was 94.03 ml/min/1.73 m² (±12.34 mL/min/1.73 m²) and the mean Psychometric Hepatic Encephalopathy Score (PHES) was 0 (±3.07).

Of the 8 participants enrolled in Cohort 2, 6 (75.0%) were men and 2 (25.0%) were women. The mean age was 60.5 years (±12.81 years) and the mean weight was 79.86 kg (±13.32 kg). The median BMI was 24.60 kg/m² (range: 22.6 to 30.3 kg/m²). All participants were White by race and not Hispanic or Latino in ethnicity. The mean Child-Pugh score of this cohort was 5.3 (±0.46). The mean eGFR for this cohort was 103.12 mL/min/1.73 m² (±15.22 mL/min/1.73 m²) and the mean PHES was-3.3 (±2.71). In this cohort slight ascites was diagnosed in 1 (12.5%) participant.

Of the 8 participants enrolled in Cohort 3, 4 (50.0%) were men and 4 (50.0%) were women. The mean age was 63.0 years (±5.45 years) and the mean weight was 75.59 kg (±20.06 kg). The median BMI was 24.75 kg/m² (range: 19.2 to 35.8 kg/m²). All participants were White by race and not Hispanic or Latino in ethnicity. The mean Child-Pugh score of this cohort was 7.1 (±0.35). The mean eGFR for this cohort was 90.63 mL/min/1.73 m² (±12.89 mL/min/1.73 m²) and the mean PHES was-4.0 (±2.88). In this cohort slight ascites was diagnosed in all 8 (100%) participants.

All the participants enrolled in the mild and moderate hepatic impairment group had active medical condition of liver cirrhosis (predominantly alcoholic/ethyl toxic liver cirrhosis). The most commonly disorders/infections in mild and moderate hepatic impairment group included hypertension in 7 participants, and ascites, hepatitis B infection, hepatitis C infection, and chronic obstructive pulmonary disease reported in 3 participants each.

Participants entered the study site on the morning of Day 1 and remained there until after collection of the 96 hours PK sample on Day 5. On Day 1, participants with normal hepatic function and participants with mild hepatic impairment received a single 20 mg oral dose (given as a 20 mg tablet) of seltorexant and participants with moderate hepatic impairment received a single 10 mg oral dose (given as a 10 mg tablet) of seltorexant, followed by serial blood sample collection and urine sample collection for measurement of plasma and urine concentrations of seltorexant and its metabolites, M12 and M16. In addition, blood samples for determination of the plasma protein binding (PPB) were collected.

Seltorexant was administered at bedtime approximately 3 hours after the start of a standard dinner, orally, with 240 mL of room-temperature noncarbonated water. Seltorexant was swallowed whole and not chewed, divided, dissolved, or crushed. Participants remained in supine position from the predose assessments until 4-hour postdose but were allowed short upright stints for pressing needs (e.g., bathroom visits) and during consumption of meal. Fluids (e.g., water) were restricted 1 hour before and 1 hour after dosing, except for the water administered with the study intervention. Participants were provided a standard dinner 3 hours before study intervention administration. The standard dinner was eaten completely within a 30-minute period or less.

Participants with hepatic impairment were instructed not to take their allowed concomitant medications for at least 2 hours before and after seltorexant administration.

Descriptive statistics were calculated for the plasma concentrations of seltorexant and its metabolites, M12 and M16 at each applicable time point per cohort, and for the derived plasma and urine PK parameters, such as:
Cₘₐₓ: maximum observed plasma analyte concentration
tₘₐₓ: time to reach the peak plasma concentration
AUCₗₐₛₜ: area under plasma analyte concentration vs. time curve from time zero to time of last measurable concentration
AUC_{∞}: area under the plasma analyte concentration versus time curve from time zero to infinite time
λ_{z}: elimination rate constant, determined by linear regression of the terminal points of the In-linear plasma concentration-time curve
t_{1/2}: terminal half-life, defined as 0.693 λ_{z}
CL/F: total clearance of drug after extravascular administration uncorrected for absolute bioavailability, calculated as Dose/AUC_{∞}
V_{d}/F: apparent volume of distribution after extravascular administration, uncorrected for absolute bioavailability
   AUC_{∞} may be calculated as AUCₗₐₛₜ+Cₗₐₛₜ/λ_{z}, where Cₗₐₛₜ is the last observed measurable (non-BQ1) plasma analyte concentration. Statistics included sample size (n), mean, standard deviation (SD), % CV, arithmetic mean, geometric mean, median, minimum, and maximum.

For comparison of mild and moderate hepatic impairment (Cohort 2 and Cohort 3) to normal hepatic function (Cohort 1), an analysis of variance (ANOVA) model was fitted to log-transformed AUCs (AUC_{∞} and AUCₗₐₛₜ) and Cₘₐₓ with hepatic impairment group as a factor. Ninety percent confidence intervals for the ratio of mean PK parameters AUCs and Cₘₐₓ for participants with mild and moderate hepatic impairment (Cohort 2 and Cohort 3) compared to participants with a normal hepatic function (Cohort 1) were constructed using the estimated least squares means and inter-participant variance from ANOVA models.

Plasma and urine samples of seltorexant, M12 (JNJ-49388599), and M16 (JNJ-51963964) were analyzed using a validated bioanalytical method using LC-MS/MS for analysis. Plasma concentrations of seltorexant, M12, and M16 were determined using lower limit of quantification (LLOQ) of 1.00 ng/mL. The range of quantitation was 1.00 to 2000 ng/ml for seltorexant, M12 and M16.

### Pharmacokinetics of Seltorexant

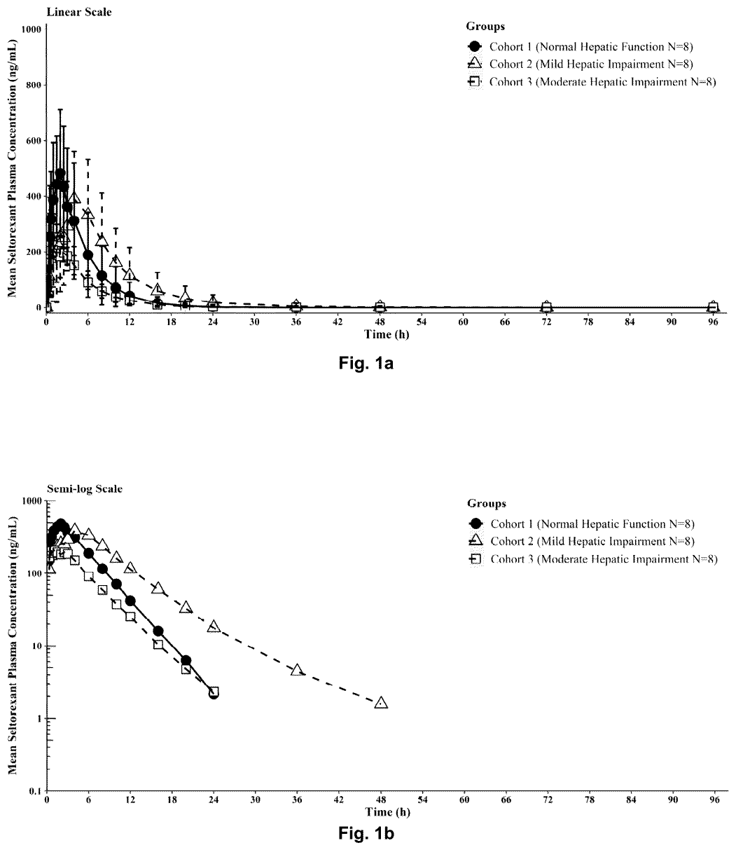

FIG. 1a shows the linear mean plasma concentration-time profiles of seltorexant after oral administration of 20 mg seltorexant in Cohort 1 and Cohort 2 and 10 mg seltorexant in Cohort 3. FIG. 1b shows the data of FIG. 1a in a semi-logarithmic scale. As shown in FIGS. 1a and 1b, following a single oral administration of 20 mg seltorexant (normal hepatic function and mild hepatic impairment) or 10 mg seltorexant (moderate hepatic impairment), the mean plasma concentration-time profiles of seltorexant declined mono-exponentially post reaching tₘₐₓ.

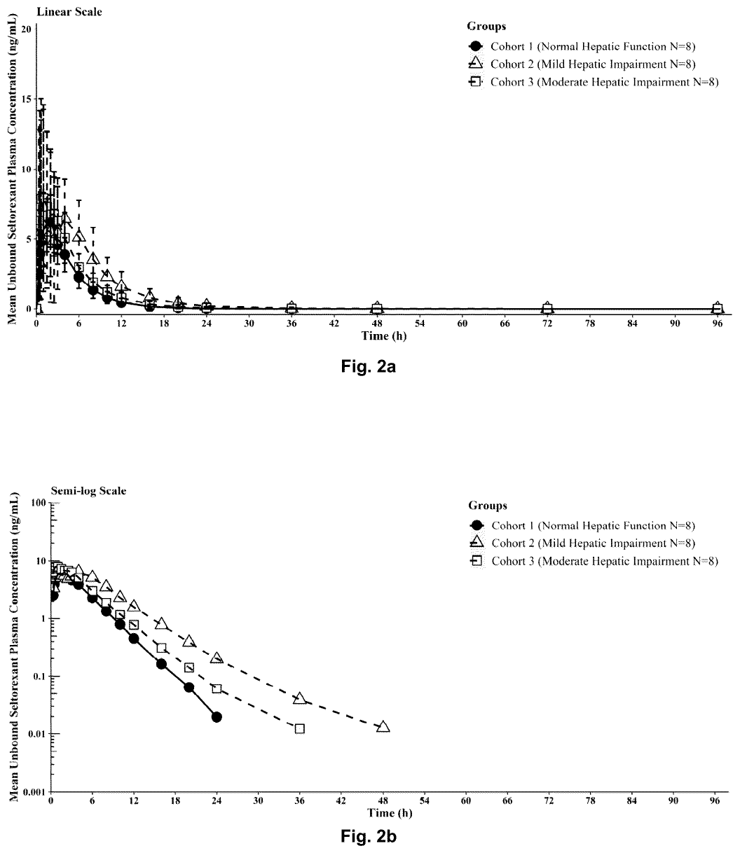

For all cohorts, 2-hours fraction unbound values were used to derive the unbound plasma concentrations and these unbound concentrations were used to derive unbound PK parameters. The mean fraction unbound values of seltorexant at 2-hours for normal hepatic function, mild hepatic impairment, and moderate hepatic impairment are 1.50%, 2.02% and 3.39%, respectively. FIG. 2a shows the linear mean plasma concentration-time profiles of unbound seltorexant after oral administration of 20 mg seltorexant in Cohort 1 and Cohort 2 and 10 mg seltorexant in Cohort 3. FIG. 2b shows the data of FIG. 2b in a semi-logarithmic scale. Following a single oral administration of 20 mg seltorexant (normal hepatic function and mild hepatic impairment) or 10 mg seltorexant (moderate hepatic impairment), the mean plasma concentration-time profiles of unbound seltorexant declined mono-exponentially post reaching tₘₐₓ.

A summary list of key PK parameters of seltorexant for all cohorts including dose normalized PK parameters is presented in Table 3.

TABLE 3

| Pharmaco- | | | | Moderate |
|---|---|---|---|---|
| kinetics | | | | Hepatic |
| parameter of | | | | Impairment |
| Seltorexant | | | | Dose |
| (mean | Normal | | Moderate | Normalized |
| (SD), tₘₐₓ: | Hepatic | Mild Hepatic | Hepatic | (Cohort |
| median | Function | Impairment | Impairment | 3 dose |
| [Min-Max]) | (Cohort 1) | (Cohort 2) | (Cohort 3) | normalized) |
| n | 8 | 8 | 8 | 8 |
| Cₘₐₓ (ng/mL) | 542 (201) | 547 (58.3) | 287 (95.4) | 574 (191) |
| tₘₐₓ (h) | 1.50 | 4.00 | 1.09 | — |
| | [0.33-2.00] | [0.50-6.00] | [0.33-3.00] | |
| AUCₗₐₛₜ | 2714 (1646) | 3718 (2032) | 1383 (240) | 2765 (481) |
| (h*ng/mL) | | | | |
| AUC_{∞} | 2720 (1647) | 3750 (2070) | 1397 (246) | 2794(492) |
| (h*ng/mL) | | | | |
| λ_{z} (1/h) | 0.265 (0.045) | 0.210 (0.073) | 0.222 (0.090) | — |
| t_{1/2} (h) | 2.68 (0.439) | 3.87 (2.00) | 3.92 (2.35) | — |
| CL/F (L/h) | 9.39 (4.41) | 6.62 (3.12) | 7.37 (1.43) | — |
| V_{d}/F (L) | 35.5 (18.6) | 31.7 (8.08) | 43.1 (33.8) | — |

Note:
Cohort 3 Dose Normalized to 20 mg

The mean PK parameters Cₘₐₓ, AUCₗₐₛₜ, and AUC_{∞} for mild hepatic impairment are 547 ng/mL, 3718 h*ng/ml, and 3750 h*ng/ml; for normal hepatic function are 542 ng/ml, 2714 h*ng/mL, and 2720 h*ng/mL, respectively. The mild hepatic impairment mean PK parameter Cₘₐₓ, was comparable, whereas AUCₗₐₛₜ and AUC_{∞} of seltorexant were higher by 1.37- and 1.38-fold compared to normal hepatic function. Elimination half-life (t_{1/2}) and median tₘₐₓ of normal hepatic function and mild hepatic impairment, were 2.68 h, 3.87 h and 1.50 h, 4.00 h, respectively.

Participants in Cohort 3 (moderate hepatic impairment) received a lower dose of 10 mg of seltorexant, therefore the PK parameters are dose normalized to 20 mg to be able to compare to the normal hepatic function group (Cohort 1). The mean PK parameters Cₘₐₓ, AUCₗₐₛₜ, and AUC_{∞} for moderate hepatic impairment (without dose normalization) are 287 ng/mL, 1383 h*ng/mL, and 1397 h*ng/mL, respectively. The dose normalized moderate hepatic impairment mean PK parameters Cₘₐₓ, AUCₗₐₛₜ, and AUC_{∞} (574 ng/ml, 2765 h*ng/ml and 2794 h*ng/mL, respectively) of seltorexant were comparable to normal hepatic function (Cₘₐₓ, AUCₗₐₛₜ, and AUC_{∞} are 542 ng/mL, 2714 h*ng/ml and 2720 h*ng/ml, respectively).

Plasma protein binding of seltorexant in participants with varying degrees of hepatic function on Day 1 at 2 hours for plasma concentrations of α-1-AGP, total protein, and albumin, per cohort are presented in Table 4.

TABLE 4

| Plasma Protein | | | |
|---|---|---|---|
| Binding for Day 1- | Normal Hepatic | Mild Hepatic | Moderate Hepatic |
| 2 h of Seltorexant | Function | Impairment | Impairment |
| (mean (SD)) | (Cohort 1) | (Cohort 2) | (Cohort 3) |
| n | 8 | 8 | 8 |
| % fᵤ | 1.50 (0.564) | 2.02 (1.37) | 3.39 (1.22) |
| % f_{b} | 98.5 (0.564) | 98.0 (1.37) | 96.6 (1.22) |
| Albumin (g/dL) | 4.04 (0.182) | 3.94 (0.231) | 3.29 (0.380) |
| α-1-AGP (mg/dL) | 68.2 (23.6) | 54.4 (16.1) | 36.0 (9.16) |
| Total Protein (g/dL) | 6.38 (0.554) | 6.44 (0.391) | 5.98 (0.453) |

The relationships between albumin, bilirubin, prothrombin time, ALP, ALT, AST, total protein and α-1-AGP to the total seltorexant PK parameters (Cₘₐₓ, AUCₗₐₛₜ, AUC_{∞}, and CL/F) were evaluated by fitting a linear regression model with the total seltorexant PK parameters (Cₘₐₓ, AUCₗₐₛₜ, AUC_{∞} and CL/F) as the dependent variable and either albumin, bilirubin, prothrombin time, ALP, ALT, AST, total protein or α-1-AGP as a linear predictor (baseline values were used). The slope of the regression line for AUCₗₐₛₜ and AUC_{∞} were found to be statistically significant for α-1-AGP (p-values were 0.0262 and 0.0283, respectively) and CL/F was found to be statistically significant for both total protein and α-1-AGP (p-values were 0.0451 and 0.0025, respectively).

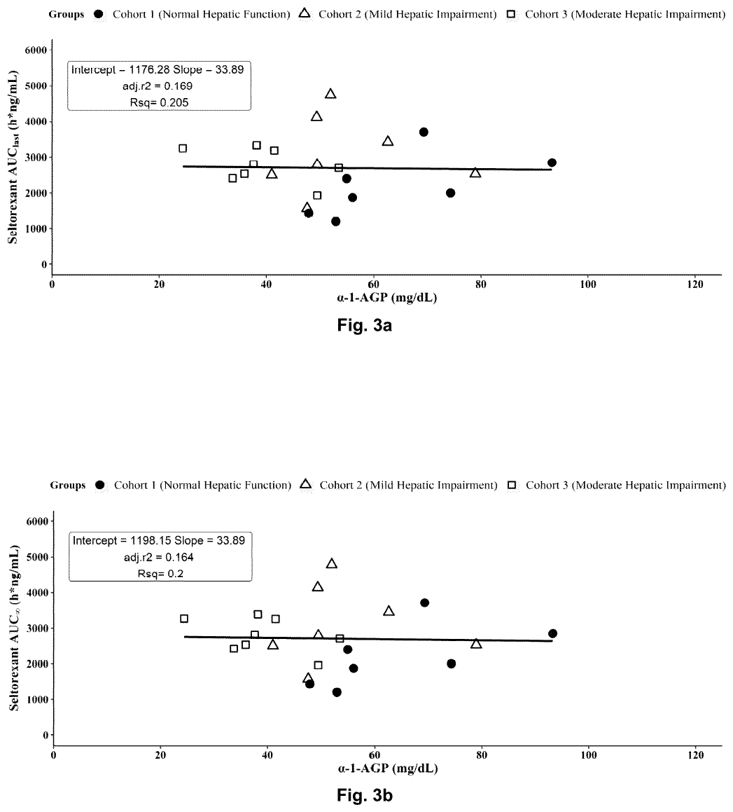

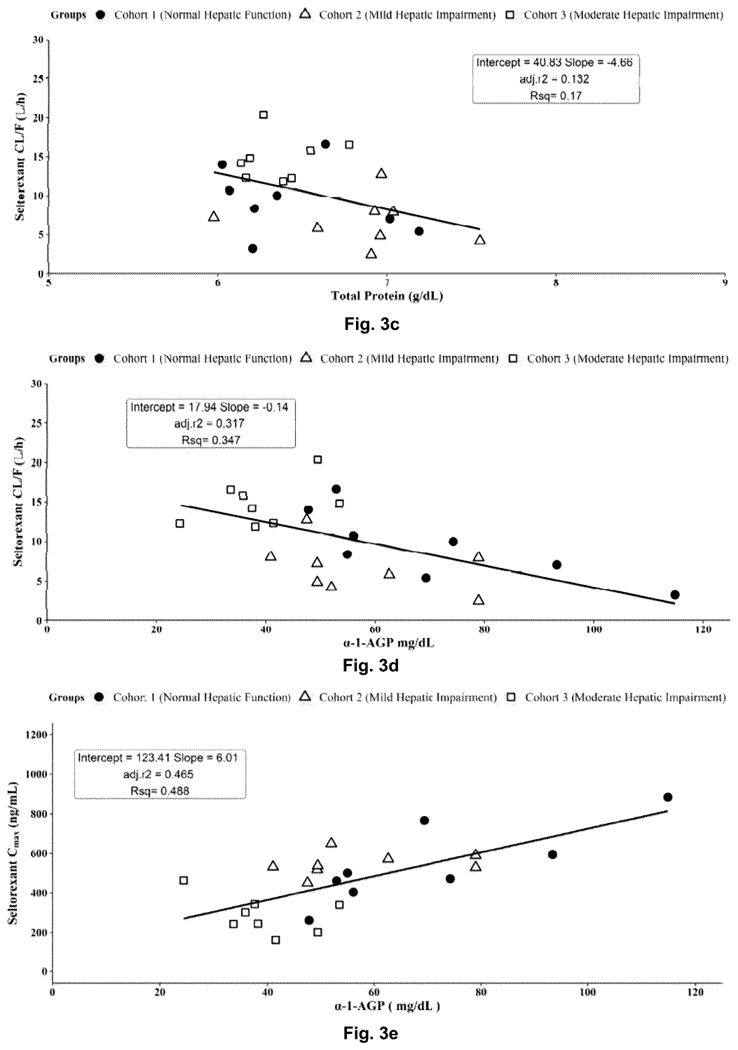

FIG. 3a shows scatter plot of seltorexant AUCₗₐₛₜ against α-1-AGP levels after oral administration of 20 mg seltorexant in Cohort 1 and Cohort 2 and 10 mg seltorexant in Cohort 3 (dose normalized to 20 mg). FIG. 3b shows scatter plot of seltorexant AUC_{∞} against α-1-AGP levels after oral administration of 20 mg seltorexant in Cohort 1 and Cohort 2 and 10 mg seltorexant in Cohort 3 (dose normalized to 20 mg). FIG. 3c shows scatter plot of seltorexant CL/F against total protein levels after oral administration of 20 mg seltorexant in Cohort 1 and Cohort 2 and 10 mg seltorexant in Cohort 3 (dose normalized to 20 mg). FIG. 3d shows scatter plot of seltorexant CL/F against α-1-AGP levels after oral administration of 20 mg seltorexant in Cohort 1 and Cohort 2 and 10 mg seltorexant in Cohort 3 (dose normalized to 20 mg). FIG. 3e shows scatter plot of total seltorexant Cₘₐₓ against α-1-AGP levels after oral administration of 20 mg seltorexant in Cohort 1 and Cohort 2 and 10 mg seltorexant in Cohort 3 (dose normalized to 20 mg).

A summary list of key PK parameters of unbound seltorexant for all cohorts including dose normalized PK parameters is presented in Table 5.

TABLE 5

| Pharmaco- | | | | Moderate |
|---|---|---|---|---|
| kinetics | | | | Hepatic |
| parameter of | | | | Impairment |
| Seltorexant | Normal | | Moderate | Dose |
| (mean (SD), | Hepatic | Mild Hepatic | Hepatic | Normalized |
| tₘₐₓ:median | Function | Impairment | Impairment | (Cohort 3 dose |
| [Min-Max]) | (Cohort 1) | (Cohort 2) | (Cohort 3) | normalized) |
| n | 8 | 8 | 8 | 8 |
| Cₘₐₓ (ng/mL) | 7.32 (1.95) | 10.6 (6.25) | 10.3 (6.15) | 20.6 (12.3) |
| AUCₗₐₛₜ (h*ng/mL) | 34.1 (9.05) | 60.2 (22.6) | 47.3 (19.1) | 94.6 (38.1) |
| AUC_{∞} (h*ng/mL) | 34.2 (9.06) | 60.6 (22.7) | 47.7 (19.1) | 95.5 (38.2) |

Note:
Cohort 3 Dose Normalized to 20 mg

The mean PK parameters C_{max,unbound}, AUC_{last,unbound}, and AUC_{∞,unbound} for mild hepatic impairment are 10.6 ng/ml, 60.2 h*ng/mL, and 60.6 h*ng/ml; for normal hepatic function are 7.32 ng/ml, 34.1 h*ng/ml, and 34.2 h*ng/mL, respectively. The mild hepatic impairment mean PK parameters of C_{max,unbound}, AUC_{last,unbound}, and AUC_{∞, unbound} of unbound seltorexant were higher by 1.45-, 1.77-, and 1.77-fold, respectively, compared to normal hepatic function.

Participants in Cohort 3 (moderate hepatic impairment) received a lower dose of 10 mg of seltorexant, therefore the PK parameters are dose normalized to 20 mg to be able to compare to the normal hepatic function group (Cohort 1). The mean PK parameters C_{max,unbound}, AUC_{last,unbound} and AUC_{∞,unbound} for moderate hepatic impairment (without dose normalization) are 10.3 ng/mL, 47.3 h*ng/mL, and 47.7 h*ng/mL, respectively. The dose normalized moderate hepatic impairment mean PK parameters C_{max,unbound}, AUC_{last,unbound} and AUC_{∞,unbound} (20.6 ng/mL, 94.6 h*ng/ml and 95.5 h*ng/mL, respectively) of unbound seltorexant were higher by 2.81-, 2.77- and 2.79-fold, respectively, compared to normal hepatic function (C_{max,unbound}, AUC_{last,unbound}, and AUC_{∞,unbound} are 7.32 ng/mL, 34.1 h*ng/ml and 34.2 h*ng/mL, respectively).

The relationships between albumin, bilirubin, prothrombin time, ALP, ALT, AST, total protein and α-1-AGP to the unbound seltorexant PK parameters (C_{max,unbound}, AUC_{last, unbound} and AUC_{∞,unbound}) were evaluated by fitting a linear regression model with the unbound seltorexant PK parameters (C_{max,unbound}, AUC_{last,unbound} and AUC_{∞,unbound}) as the dependent variable and either albumin, bilirubin, prothrombin time, ALP, ALT, AST, total protein or α-1-AGP as a linear predictor (baseline values were used). The slope of the regression line for C_{max, unbound} was found to be statistically significant for albumin, prothrombin time and α-1-AGP (p-values were 0.0099, 0.0005 and 0.0009, respectively); AUC_{last,unbound} and AUC_{∞,unbound} were significant for albumin, prothrombin time, ALP, α-1-AGP (p-values were 0.0006, 0.0025, 0.0248, 0.0006 and 0.0005, 0.0025, 0.0238, 0.0006, respectively).

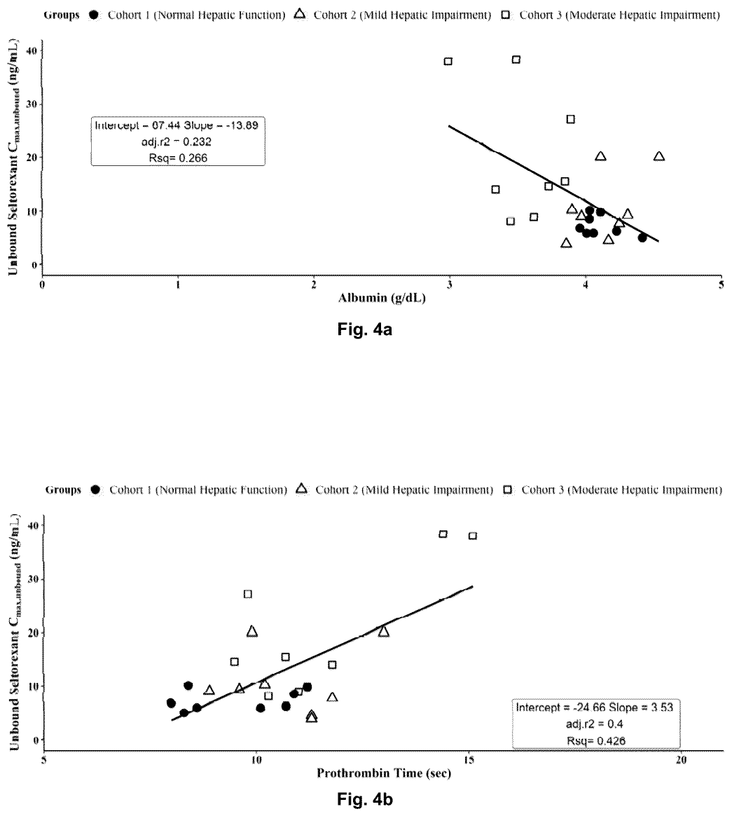

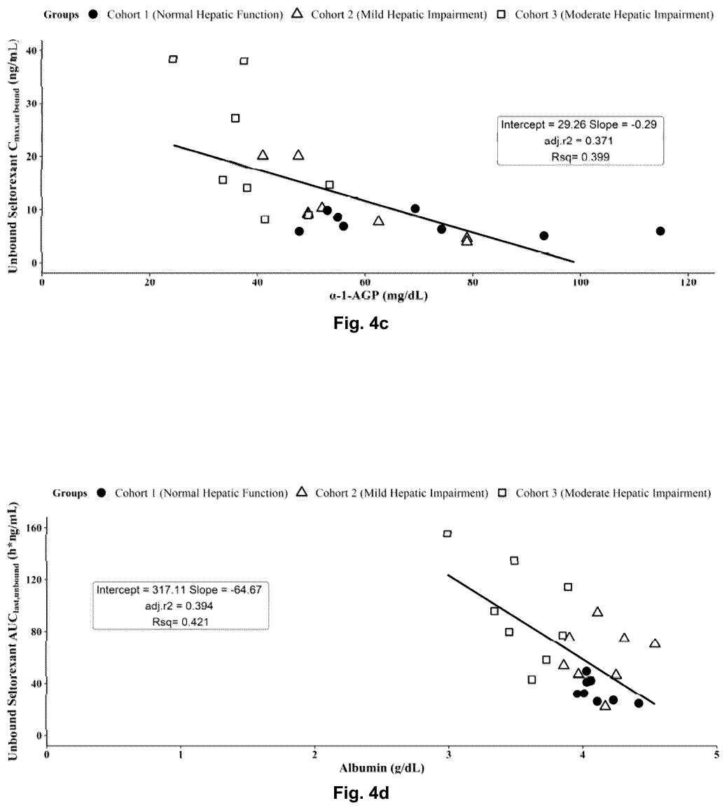

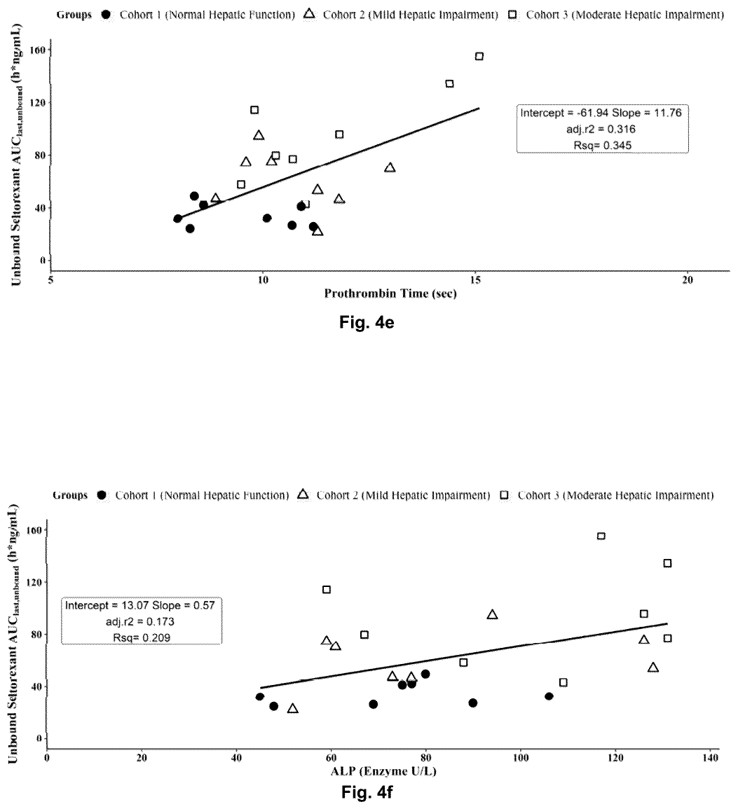

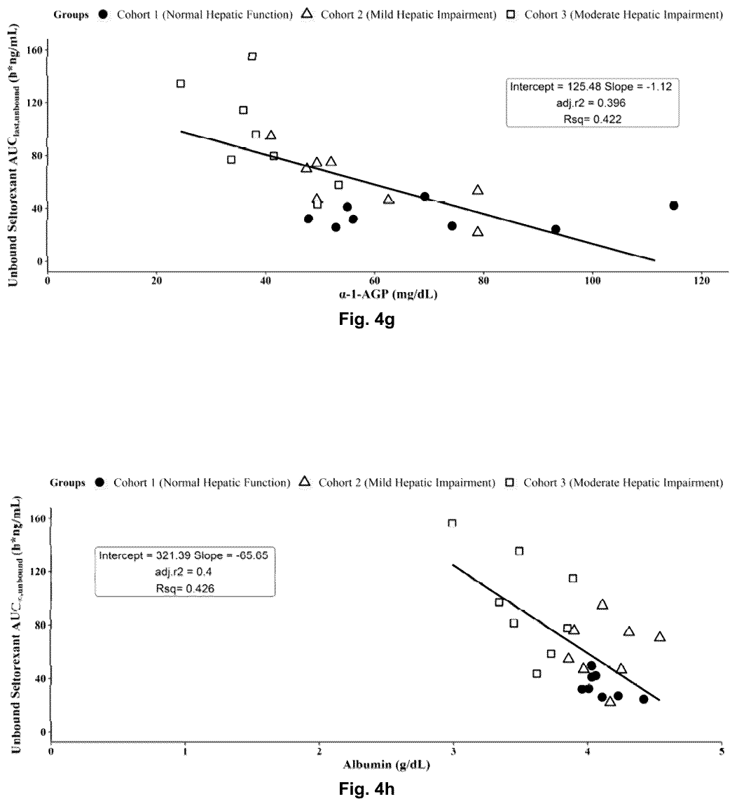

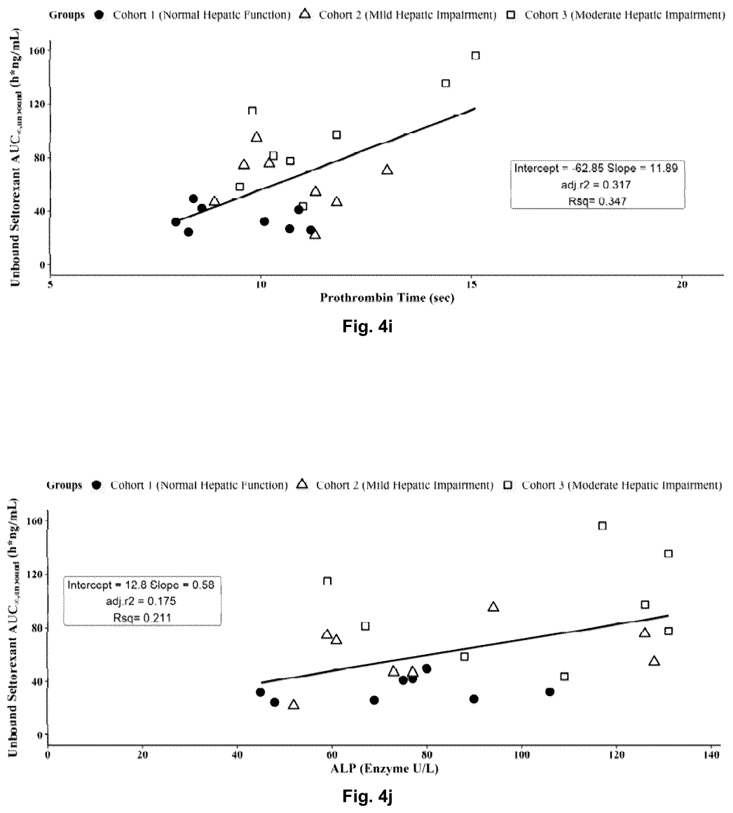

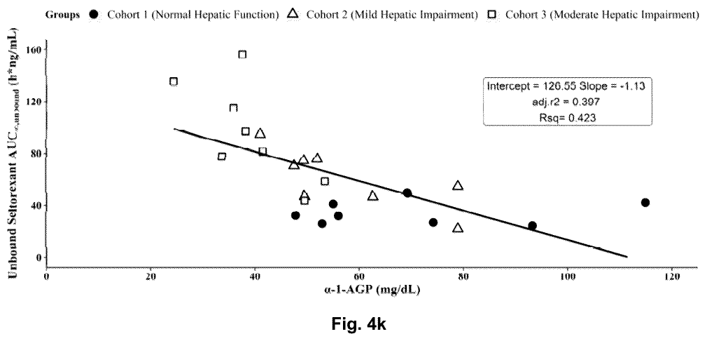

FIG. 4a shows scatter plot of unbound seltorexant C_{max,unbound} against albumin levels after oral administration of 20 mg seltorexant in Cohort 1 and Cohort 2 and 10 mg seltorexant in Cohort 3 (dose normalized to 20 mg). FIG. 4b shows scatter plot of unbound seltorexant C_{max,unbound} against prothrombin time after oral administration of 20 mg seltorexant in Cohort 1 and Cohort 2 and 10 mg seltorexant in Cohort 3 (dose normalized to 20 mg). FIG. 4c shows scatter plot of unbound seltorexant C_{max,unbound} against α-1-AGP levels after oral administration of 20 mg seltorexant in Cohort 1 and Cohort 2 and 10 mg seltorexant in Cohort 3 (dose normalized to 20 mg). FIG. 4d shows scatter plot of AUC_{last,unbound} against albumin levels after oral administration of 20 mg seltorexant in Cohort 1 and Cohort 2 and 10 mg seltorexant in Cohort 3 (dose normalized to 20 mg). FIG. 4e shows scatter plot of AUC_{last,unbound} against prothrombin time after oral administration of 20 mg seltorexant in Cohort 1 and Cohort 2 and 10 mg seltorexant in Cohort 3 (dose normalized to 20 mg). FIG. 4f shows scatter plot of AUC_{last, unbound} against ALP levels after oral administration of 20 mg seltorexant in Cohort 1 and Cohort 2 and 10 mg seltorexant in Cohort 3 (dose normalized to 20 mg). FIG. 4g shows scatter plot of AUC_{last,unbound} against α-1-AGP levels after oral administration of 20 mg seltorexant in Cohort 1 and Cohort 2 and 10 mg seltorexant in Cohort 3 (dose normalized to 20 mg). FIG. 4h shows scatter plot of AUC_{∞,unbound} against albumin levels after oral administration of 20 mg seltorexant in Cohort 1 and Cohort 2 and 10 mg seltorexant in Cohort 3 (dose normalized to 20 mg). FIG. 4i shows scatter plot of AUC_{∞,unbound} against prothrombin time after oral administration of 20 mg seltorexant in Cohort 1 and Cohort 2 and 10 mg seltorexant in Cohort 3 (dose normalized to 20 mg). FIG. 4j shows scatter plot of AUC_{∞,unbound} against ALP levels after oral administration of 20 mg seltorexant in Cohort 1 and Cohort 2 and 10 mg seltorexant in Cohort 3 (dose normalized to 20 mg). FIG. 4k shows scatter plot of AUC_{∞,unbound} against α-1-AGP levels after oral administration of 20 mg seltorexant in Cohort 1 and Cohort 2 and 10 mg seltorexant in Cohort 3 (dose normalized to 20 mg).

A summary of statistical results of seltorexant for mild hepatic impairment versus normal hepatic function is presented in Table 6. Specifically, Table 6 shows summarized statistical results for estimated ratio of geometric means and 90% confidence interval of seltorexant exposure parameters after oral administration of 20 mg seltorexant in Cohort 1 and Cohort 2.

TABLE 6

| | | | | Ratio of | | |
|---|---|---|---|---|---|---|
| | | | | Geometric | | |
| | PK | Cohort 1 | Cohort 2 | Means | 90% CI | |
| Analyte | Parameter | (Reference) | (Test) | (%) | (%) | % CV |
| SELTOREXANT | Cₘₐₓ | 510 | 544 | 106.63 | 82.50-137.81 | 30.5 |
| | (ng/mL) | | | | | |
| | AUCₗₐₛₜ | 2375 | 3323 | 139.89 | 96.35-203.10 | 45.5 |
| | (h*ng/mL) | | | | | |
| | AUC_{∞} | 2381 | 3345 | 140.44 | 96.65-204.08 | 45.6 |
| | (h*ng/mL) | | | | | |

N = 8 participants per cohort
Note:
Log transformed PK parameters were analyzed by an analysis of variance (ANOVA) model for Cohort as a factor and the results were back-transformed using anti-logarithm.
N: number of participants who completed at least one treatment and for which an evaluable PK parameter could be obtained were included.
% CV = 100* (sqrt(exp(MSE) − 1).

Based on the geometric mean ratios (GMR) of seltorexant, mild hepatic impairment Cmax and AUCs (AUCₗₐₛₜ and AUC_{∞}) were approximately higher by 1.07-fold and 1.40-fold, respectively, compared to normal hepatic function. The % CV is 30.5% for Cₘₐₓ, 45.5% and 45.6% for AUCs (AUCₗₐₛₜ and AUC_{∞}). Point estimate ratios (90% CI) for Cₘₐₓ, AUCₗₐₛₜ, and AUC_{∞} of seltorexant for mild hepatic impairment versus normal hepatic function were 106.63% (82.50%-137.81%), 139.89% (96.35%-203.10%) and 140.44% (96.65%-204.08%), respectively.

Participants in Cohort 3 received a lower dose of 10 mg of seltorexant, therefore the PK parameters are dose normalized to 20 mg to be able to compare to the normal hepatic function (Cohort 1). A summary of statistical results of seltorexant for dose normalized moderate hepatic impairment versus normal hepatic function is presented in Table 7. Specifically, Table 7 shows summarized statistical results for estimated ratio of geometric means and 90% confidence interval of seltorexant exposure parameters after oral administration of 20 mg seltorexant in Cohort 1 and 10 mg seltorexant in Cohort 3 dose normalized to 20 mg.

TABLE 7

| | | | | Ratio of | | |
|---|---|---|---|---|---|---|
| | | | | Geometric | | |
| | PK | Cohort 1 | Cohort 3 | Means | 90% CI | |
| Analyte | Parameter | (Reference) | (Test) | (%) | (%) | % CV |
| SELTOREXANT | Cₘₐₓ | 510 | 547 | 107.28 | 83.00-138.65 | 30.5 |
| | (ng/mL) | | | | | |
| | AUCₗₐₛₜ | 2375 | 2726 | 114.79 | 79.06-166.66 | 45.5 |
| | (h*ng/mL) | | | | | |
| | AUC_{∞} | 2381 | 2754 | 115.66 | 79.59-168.06 | 45.6 |
| | (h*ng/mL) | | | | | |

N = 8 participants per cohort
Note:
Log transformed PK parameters were analyzed by an analysis of variance (ANOVA) model for Cohort as a factor and the results were back-transformed using anti-logarithm.
N: number of participants who completed at least one treatment and for which an evaluable PK parameter could be obtained were included.
% CV = 100* (sqrt(exp(MSE) − 1).

Based on the GMR of seltorexant, dose normalized moderate hepatic impairment Cₘₐₓ, AUCₗₐₛₜ, and AUC_{∞} were approximately higher by 1.07-fold, 1.15-fold, and 1.16-fold, respectively, compared to normal hepatic function. The % CV for Cₘₐₓ, AUCₗₐₛₜ, and AUC_{∞} were 30.5%, 45.5% and 45.6%, respectively. Point estimate ratios (90% CI) for Cₘₐₓ, AUCₗₐₛₜ, and AUC_{∞} of seltorexant for dose normalized moderate hepatic impairment versus normal hepatic function were 107.28% (83.00%-138.65%), 114.79% (79.06%-166.66%), and 115.66% (79.59%-168.06%), respectively.

A summary of statistical results of unbound seltorexant for mild hepatic impairment versus normal hepatic function is presented in Table 8. Specifically, Table 8 provides summarized statistical results for estimated ratio of geometric means and 90% confidence interval of unbound seltorexant exposure parameters after oral administration of 20 mg seltorexant in Cohort 1 and Cohort 2.

TABLE 8

| | | | | Ratio of | | |
|---|---|---|---|---|---|---|
| | | | | Geometric | | |
| | PK | Cohort 1 | Cohort 2 | Means | 90% CI | |
| Analyte | Parameter | (Reference) | (Test) | (%) | (%) | % CV |
| UNBOUND | Cₘₐₓ | 7.10 | 9.10 | 128.24 | 82.49-199.35 | 54.8 |
| SELTOREXANT | (ng/mL) | | | | | |
| | AUCₗₐₛₜ | 33.1 | 55.6 | 168.24 | 120.10-235.67 | 40.7 |
| | (h*ng/mL) | | | | | |
| | AUC_{∞} | 33.2 | 56.0 | 168.91 | 120.67-236.43 | 40.6 |
| | (h*ng/mL) | | | | | |

N = 8 participants per cohort
Note:
Log transformed PK parameters were analyzed by an analysis of variance (ANOVA) model for Cohort as a factor and the results were back-transformed using anti-logarithm.
N: number of participants who completed at least one treatment and for which an evaluable PK parameter could be obtained were included.
% CV = 100* (sqrt(exp(MSE) − 1).

Based on the GMR of unbound seltorexant, mild hepatic impairment Cₘₐₓ, unbound, AUC_{last,unbound} and AUC_{∞,unbound} were approximately higher by 1.28-fold, 1.68-fold, and 1.69-fold, respectively, compared to normal hepatic function. The % CV is 54.8% for C_{max,unbound}, 40.7% and 40.6% for AUCs (AUC_{last,unbound} and AUC_{∞,unbound}). Point estimate ratios (90% CI) for C_{max,unbound}, AUC_{last,unbound} and AUC_{∞,unbound} of unbound seltorexant for mild hepatic impairment versus normal hepatic function were 128.24% (82.49%-199.35%), 168.24% (120.10%-235.67%), and 168.91% (120.67%-236.43%), respectively.

Participants in Cohort 3 received a lower dose of 10 mg of seltorexant, therefore the PK parameters are dose normalized to 20 mg to be able to compare to the normal hepatic function (Cohort 1). A summary of statistical results of unbound seltorexant for dose normalized moderate hepatic impairment versus normal hepatic function is presented in Table 9. Specifically, Table 9 provides summarized statistical results for estimated ratio of geometric means and 90% confidence interval of unbound seltorexant exposure parameters after oral administration of 20 mg seltorexant in Cohort 1 and 10 mg seltorexant in Cohort 3 (dose normalized to 20 mg).

TABLE 9

| | | | | Ratio of | | |
|---|---|---|---|---|---|---|
| | | | | Geometric | | |
| | PK | Cohort 1 | Cohort 3 | Means | 90% CI | |
| Analyte | Parameter | (Reference) | (Test) | (%) | (%) | % CV |
| UNBOUND | Cₘₐₓ | 7.10 | 17.6 | 247.61 | 159.29-384.93 | 54.8 |
| SELTOREXANT | (ng/mL) | | | | | |
| | AUCₗₐₛₜ | 33.1 | 87.6 | 264.95 | 189.14-371.13 | 40.7 |
| | (h*ng/mL) | | | | | |
| | AUC_{∞} | 33.2 | 88.5 | 266.96 | 190.71-373.68 | 40.6 |
| | (h*ng/mL) | | | | | |

N = 8 participants per cohort
Note:
Log transformed PK parameters were analyzed by an analysis of variance (ANOVA) model for Cohort as a factor and the results were back-transformed using anti-logarithm.
N: number of participants who completed at least one treatment and for which an evaluable PK parameter could be obtained were included.
% CV = 100* (sqrt(exp(MSE) − 1).

Based on the GMR of unbound seltorexant, dose normalized moderate hepatic impairment C_{max,unbound}, AUC_{last,unbound} and AUC_{∞,unbound} were approximately higher by 2.48-fold, 2.65-fold, and 2.67-fold, respectively, compared to normal hepatic function. The % CV is 54.8% for C_{max,unbound}, 40.7% and 40.6% for AUCs (AUC_{last,unbound} and AUC_{∞,unbound}). Point estimate ratios (90% CI) for C_{max,unbound}, AUC_{last,unbound} and AUC unbound of unbound seltorexant for dose normalized moderate hepatic impairment versus normal hepatic function were 247.61% (159.29%-384.93%), 264.95% (189.14%-371.13%) and 266.96% (190.71%-373.68%), respectively.

### Pharmacokinetics of M12

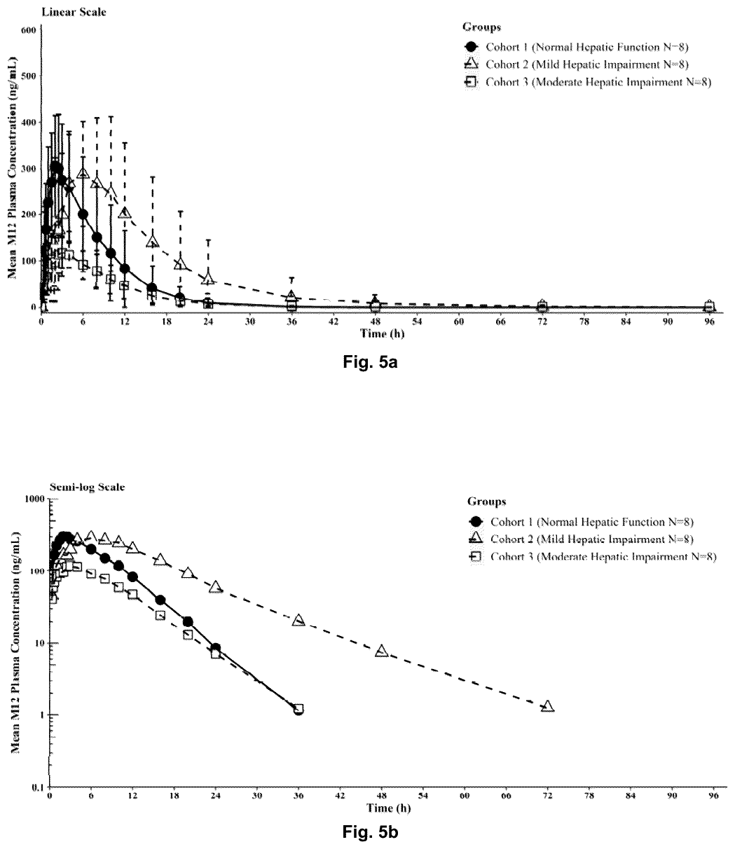

FIG. 5a shows the linear mean plasma concentration-time profiles of M12 after oral administration of 20 mg seltorexant in Cohort 1 and Cohort 2 and 10 mg seltorexant in Cohort 3. FIG. 5b shows the data of FIG. 5a in a semi-logarithmic scale. As shown in FIGS. 1a and 1b, following a single oral administration of 20 mg seltorexant (normal hepatic function and mild hepatic impairment) or 10 mg seltorexant (moderate hepatic impairment), the mean plasma concentration-time profiles of M12 declined mono-exponentially post reaching tₘₐₓ.

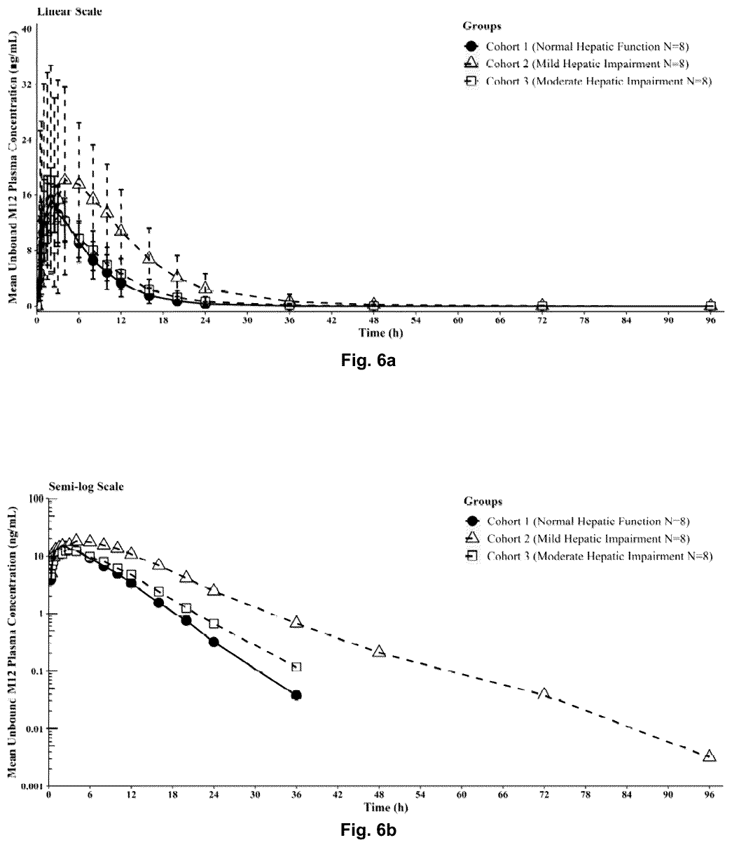

For all cohorts, 2-hours fraction unbound values were used to derive the unbound plasma concentrations and these unbound concentrations were used to derive unbound PK parameters. The mean fraction unbound values of M12 at 2-hours for normal hepatic function, mild hepatic impairment, and moderate hepatic impairment are 5.45%, 7.07% and 11.4%, respectively. FIG. 6a shows the linear mean plasma concentration-time profiles of unbound M12 after oral administration of 20 mg seltorexant in Cohort 1 and Cohort 2 and 10 mg seltorexant in Cohort 3. FIG. 6b shows the data of FIG. 6a in a semi-logarithmic scale. Following a single oral administration of 20 mg seltorexant (normal hepatic function and mild hepatic impairment) or 10 mg seltorexant (moderate hepatic impairment), the mean plasma concentration-time profiles of unbound M12 declined mono-exponentially post reaching tₘₐₓ.

A summary list of key PK parameters of M12 for all cohorts including dose normalized PK parameters is presented in Table 10.

TABLE 10

| Pharmaco- | | | | Moderate |
|---|---|---|---|---|
| kinetics | | | | Hepatic |
| parameter of | | | | Impairment |
| Seltorexant | Normal | | Moderate | Dose |
| (mean (SD), | Hepatic | Mild Hepatic | Hepatic | Normalized |
| tₘₐₓ:median | Function | Impairment | Impairment | (Cohort 3 dose |
| [Min-Max]) | (Cohort 1) | (Cohort 2) | (Cohort 3) | normalized) |
| n | 8 | 8 | 8 | 8 |
| Cₘₐₓ (ng/mL) | 336 (111) | 368 (107) | 131 (34.1) | 261 (68.2) |
| tₘₐₓ (h) | 2.00 | 5.00 [0.50- | 2.25 | — |
| | [0.50-4.00] | 10.00] | [1.00-8.00] | |
| AUCₗₐₛₜ | 2689 (1644) | 4833 (3872) | 1277 (476) | 2553 (952) |
| (h*ng/mL) | | | | |
| AUC_{∞} | 2703 (1651) | 4857 (3868) | 1292 (482) | 2584 (964) |
| (h*ng/mL) | | | | |
| λ_{z} (1/h) | 0.209 (0.043) | 0.155 (0.049) | 0.182 (0.053) | — |
| t_{1/2} (h) | 3.44 (0.690) | 4.99 (1.96) | 4.07 (1.07) | — |

Note:
Cohort 3 Dose Normalized to 20 mg

The mean M12 PK parameters Cₘₐₓ, AUCₗₐₛₜ, and AUC_{∞} for mild hepatic impairment are 368 ng/mL, 4833 h*ng/mL, and 4857 h*ng/ml; for normal hepatic function are 336 ng/mL, 2689 h*ng/mL, and 2703 h*ng/mL, respectively. The mild hepatic impairment mean PK parameter Cₘₐₓ, was comparable, whereas both AUCₗₐₛₜ and AUC_{∞} of M12 were higher by 1.80-fold, compared to normal hepatic function. Elimination half-life (_{t1/2}) and median tₘₐₓ of normal hepatic function and mild hepatic impairment, were 3.44 h, 4.99 h and 2.00 h, 5.00 h, respectively.

Participants in Cohort 3 (moderate hepatic impairment) received a lower dose of 10 mg of seltorexant, therefore the PK parameters are dose normalized to 20 mg to be able to compare to the normal hepatic function group (Cohort 1). The mean M12 PK parameters Cₘₐₓ, AUCₗₐₛₜ, and AUC_{∞} for moderate hepatic impairment (without dose normalization) are 131 ng/mL, 1277 h*ng/mL, and 1292 h*ng/mL, respectively. The dose normalized moderate hepatic impairment mean PK parameter Cₘₐₓ, was 0.78-fold, whereas AUCₗₐₛₜ and AUC_{∞} (261 ng/ml, 2553 h*ng/mL, and 2584 h*ng/mL, respectively) of M12 were comparable to normal hepatic function (Cₘₐₓ, AUCₗₐₛₜ, and AUC_{∞} are 336 ng/ml, 2689 h*ng/ml and 2703 h*ng/ml, respectively). Elimination half-life (t_{1/2}) and median tₘₐₓ of normal hepatic function, moderate hepatic impairment were 3.44 h, 4.07 h and 2.00 h, 2.25 h, respectively.

Plasma protein binding of M12 in participants with varying degrees of hepatic function on Day 1 at 2 hours for plasma concentrations of α-1-AGP, total protein, and albumin, per cohort is presented in Table 11.

TABLE 11

| Plasma Protein | | | |
|---|---|---|---|
| Binding for Day 1- | Normal Hepatic | Mild Hepatic | Moderate Hepatic |
| 2 h of Metabolite | Function | Impairment | Impairment |
| M12 (mean (SD)) | (Cohort 1) | (Cohort 2) | (Cohort 3) |
| n | 8 | 8 | 8 |
| % fᵤ | 5.45 (1.91) | 7.07 (4.58) | 11.4 (3.84) |
| % f_{b} | 94.6 (1.91) | 92.9 (4.58) | 88.6 (3.84) |
| Albumin (g/dL) | 4.04 (0.182) | 3.94 (0.231) | 3.29 (0.380) |
| α-1-AGP (mg/dL) | 68.2 (23.6) | 54.4 (16.1) | 36.0 (9.16) |
| Total Protein (g/dL) | 6.38 (0.554) | 6.44 (0.391) | 5.98 (0.453) |

The relationships between albumin, bilirubin, prothrombin time, ALP, ALT, AST, total protein and α-1-AGP to the M12 PK parameters (Cₘₐₓ, AUCₗₐₛₜ, and AUC_{∞}) were evaluated by fitting a linear regression model with the M12 PK parameters (Cₘₐₓ, AUCₗₐₛₜ, and AUC_{∞}) as the dependent variable and either albumin, bilirubin, prothrombin time, ALP, ALT, AST, total protein or α-1-AGP as a linear predictor (baseline values were used). The slope of the regression line for AUCₗₐₛₜ and AUC_{∞} were found to be statistically significant for α-1-AGP (p-values were 0.0491 and 0.0489, respectively) and Cₘₐₓ was found to be statistically significant for both total protein and α-1-AGP (p-values were 0.0038 and 0.0011, respectively).

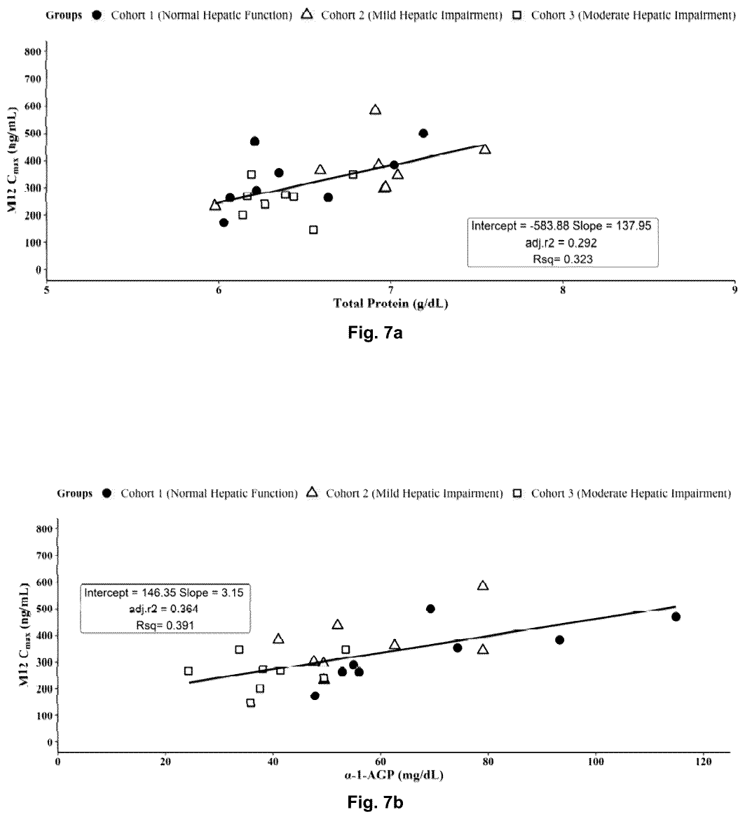

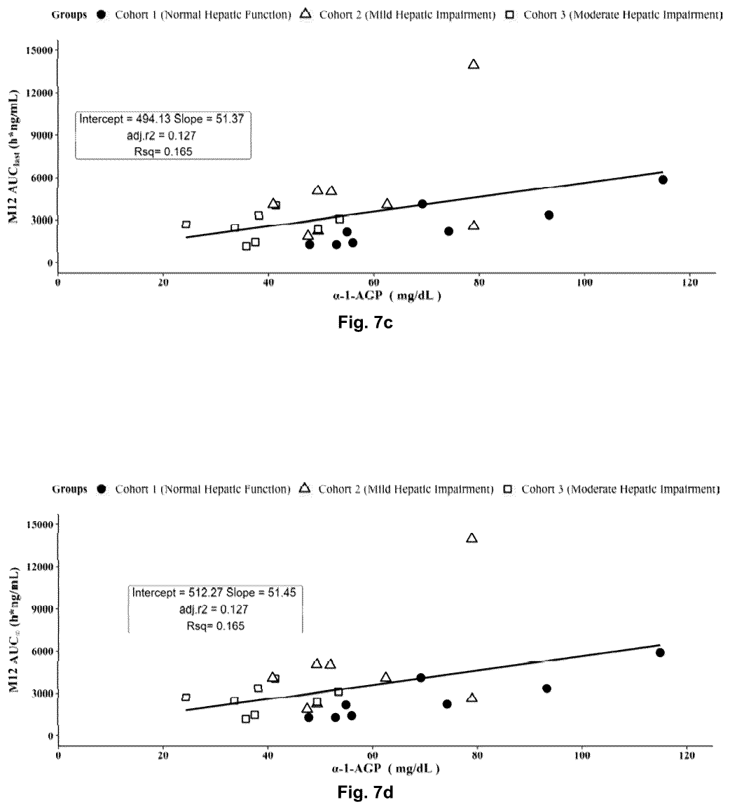

FIG. 7a shows scatter plot of M12 Cₘₐₓ against total protein levels after oral administration of 20 mg seltorexant in Cohort 1 and Cohort 2 and 10 mg seltorexant in Cohort 3 (dose normalized to 20 mg). FIG. 7b shows scatter plot of M12 Cₘₐₓ against α-1-AGP levels after oral administration of 20 mg seltorexant in Cohort 1 and Cohort 2 and 10 mg seltorexant in Cohort 3 (dose normalized to 20 mg). FIG. 7c shows scatter plot of M12 AUCₗₐₛₜ against α-1-AGP levels after oral administration of 20 mg seltorexant in Cohort 1 and Cohort 2 and 10 mg seltorexant in Cohort 3 (dose normalized to 20 mg). FIG. 7d shows scatter plot of M12 AUC_{∞} against α-1-AGP levels after oral administration of 20 mg seltorexant in Cohort 1 and Cohort 2 and 10 mg seltorexant in Cohort 3 (dose normalized to 20 mg).

A summary list of key PK parameters of unbound M12 for all cohorts including dose normalized PK parameters is presented in Table 12.

TABLE 12

| | | | | Moderate |
|---|---|---|---|---|
| Pharmacokinetics | | | | Hepatic |
| parameter of | | | | Impairment |
| unbound | Normal | | Moderate | Dose |
| Metabolite M12 | Hepatic | Mild Hepatic | Hepatic | Normalized |
| (mean | Function | Impairment | Impairment | (Cohort 3 dose |
| (SD) | (Cohort 1) | (Cohort 2) | (Cohort 3) | normalized) |
| n | 8 | 8 | 8 | 8 |
| Cₘₐₓ (ng/mL) | 16.7 (4.00) | 24.4 (15.0) | 14.2 (3.97) | 28.5 (7.94) |
| AUCₗₐₛₜ (h*ng/mL) | 123 (38.0) | 268 (138) | 134 (38.0) | 268 (76.1) |
| AUC_{∞} (h*ng/mL) | 123 (38.1) | 270 (138) | 136 (38.0) | 271 (76.0) |

Note:
Cohort 3 Dose Normalized to 20 mg

The mean PK parameters C_{max,unbound}, AUC_{last,unbound} and AUC_{∞,unbound} for mild hepatic impairment are 24.4 ng/mL, 268 h*ng/ml, and 270 h*ng/mL; for normal hepatic function are 16.7 ng/ml, 123 h*ng/mL, and 123 h*ng/mL, respectively. The mild hepatic impairment mean PK parameters of C_{max,unbound}, AUC_{last,unbound}, and AUC_{∞,unbound} of unbound M12 were higher by 1.46-, 2.18-, and 2.20-fold, respectively, compared to normal hepatic function.

Participants in Cohort 3 (moderate hepatic impairment) received a lower dose of 10 mg of seltorexant, therefore the PK parameters are dose normalized to 20 mg to be able to compare to the normal hepatic function group (Cohort 1). The mean PK parameters C_{max,unbound}, AUC_{last,unbound} and AUC_{∞,unbound} for moderate hepatic impairment (without dose normalization) are 14.2 ng/mL, 134 h*ng/mL, and 136 h*ng/mL, respectively. The dose normalized moderate hepatic impairment mean PK parameters C_{max,unbound}, AUC_{last,unbound} and AUC_{∞, unbound} (28.5 ng/mL, 268 h*ng/ml and 271 h*ng/mL, respectively) of unbound M12 were higher by 1.71, 2.18 and 2.20-fold, respectively, compared to normal hepatic function (C_{max,unbound}, AUCₗₐₛₜ, unbound and AUC_{∞,unbound} are 16.7 ng/mL, 123 h*ng/mL, and 123 h*ng/ml, respectively).

The relationships between albumin, bilirubin, prothrombin time, ALP, ALT, AST, total protein, and α-1-AGP to the unbound M12 PK parameters (C_{max,unbound}, AUC_{last,unbound} and AUC_{∞,unbound}) were evaluated by fitting a linear regression model with the unbound M12 PK parameters (C_{max,unbound}, AUC_{last,unbound} and AUC_{∞,unbound}) as the dependent variable and either albumin, bilirubin, prothrombin time, ALP, ALT, AST, total protein or α-1-AGP as a linear predictor (baseline values were used). The slope of the regression line for AUC_{last,unbound} and AUC_{∞,unbound} were found to be statistically significant for ALP, α-1-AGP (p-values were 0.0165, 0.0248 and 0.0163, 0.0238, respectively) and C_{max,unbound} was found to be statistically significant for both prothrombin time and α-1-AGP (p-values were 0.0154 and 0.0014, respectively).

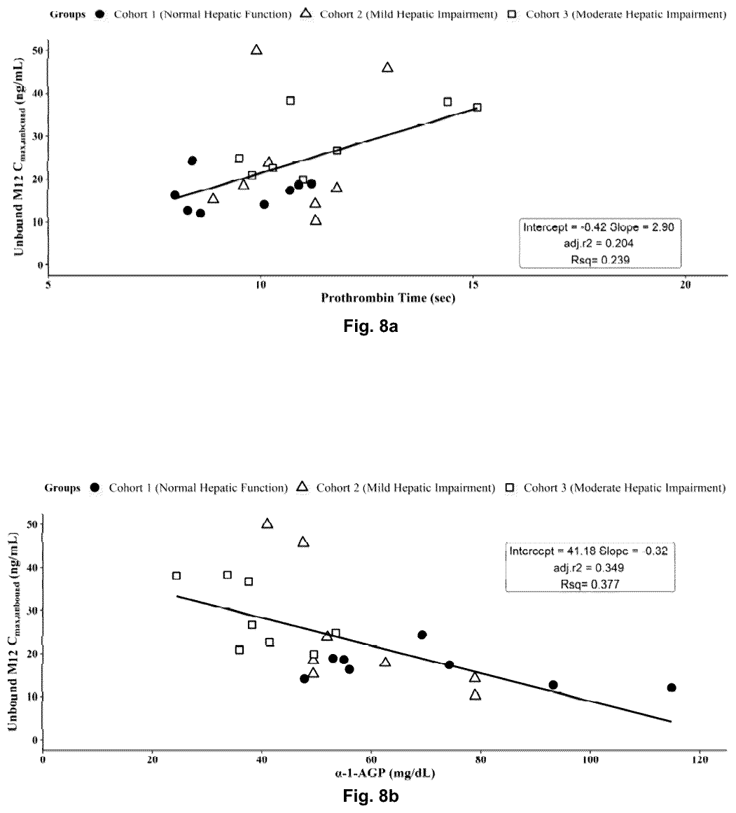

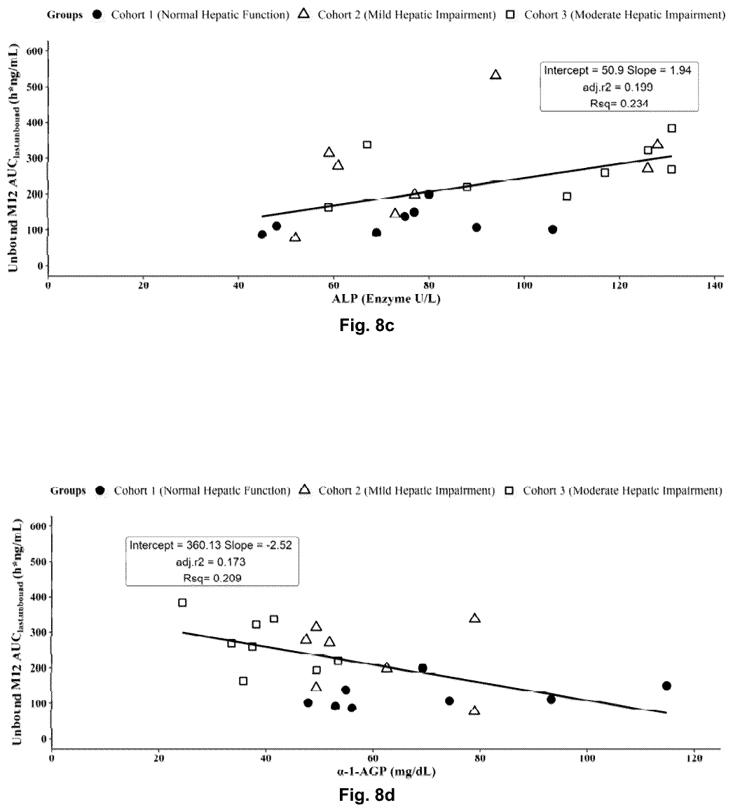

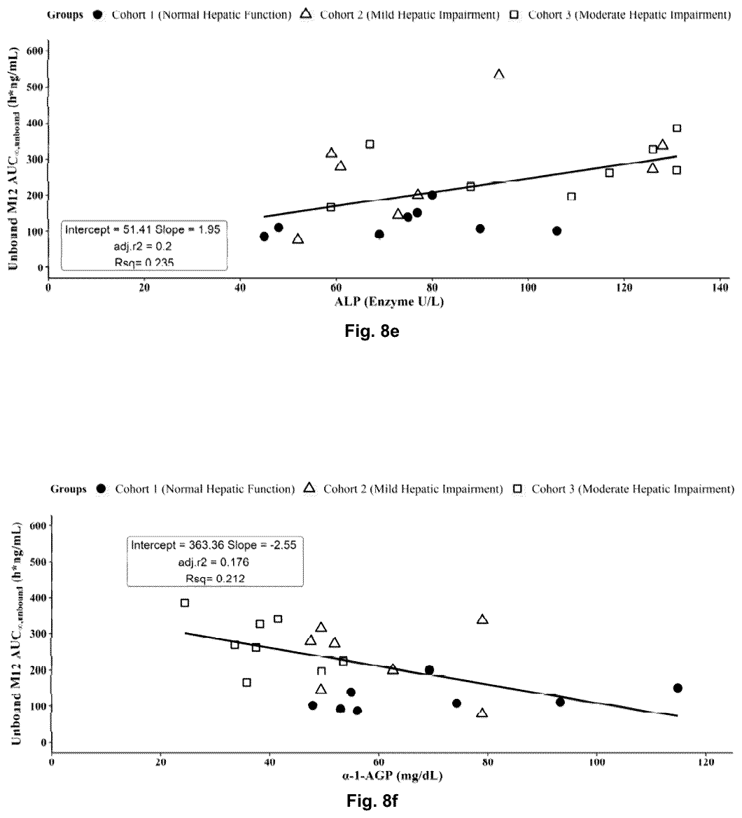

FIG. 8a shows scatter plot of unbound M12 C_{max,unbound} against prothrombin time after oral administration of 20 mg seltorexant in Cohort 1 and Cohort 2 and 10 mg seltorexant in Cohort 3 (dose normalized to 20 mg). FIG. 8b shows scatter plot of unbound M12 C_{max,unbound} against α-1-AGP levels after oral administration of 20 mg seltorexant in Cohort 1 and Cohort 2 and 10 mg seltorexant in Cohort 3 (dose normalized to 20 mg). FIG. 8c shows scatter plot of unbound M12 AUC_{last,unbound} against ALP levels after oral administration of 20 mg seltorexant in Cohort 1 and Cohort 2 and 10 mg seltorexant in Cohort 3 (dose normalized to 20 mg). FIG. 8d shows scatter plot of unbound M12 AUC_{last,unbound} against α-1-AGP levels after oral administration of 20 mg seltorexant in Cohort 1 and Cohort 2 and 10 mg seltorexant in Cohort 3 (dose normalized to 20 mg). FIG. 8e shows scatter plot of unbound M12 AUC_{∞,unbound} against ALP levels after oral administration of 20 mg seltorexant in Cohort 1 and Cohort 2 and 10 mg seltorexant in Cohort 3 (dose normalized to 20 mg). FIG. 8f shows scatter plot of unbound M12 AUC_{∞,unbound} against α-1-AGP levels after oral administration of 20 mg seltorexant in Cohort 1 and Cohort 2 and 10 mg seltorexant in Cohort 3 (dose normalized to 20 mg).

A summary of statistical results of M12 for mild hepatic impairment versus normal hepatic function is presented in Table 13. Specifically, Table 13 shows summarized statistical results for estimated ratio of geometric means and 90% confidence interval of M12 exposure parameters after oral administration of 20 mg seltorexant in Cohort 1 and Cohort 2.

TABLE 13

| | | | | Ratio of | | |
|---|---|---|---|---|---|---|
| | | | | Geometric | | |
| | PK | Cohort 1 | Cohort 2 | Means | 90% CI | |
| Analyte | Parameter | (Reference) | (Test) | (%) | (%) | % CV |
| M12 | Cₘₐₓ | 320 | 355 | 111.23 | 85.48-144.72 | 31.3 |
| | (ng/mL) | | | | | |
| | AUCₗₐₛₜ | 2310 | 3954 | 171.17 | 106.25-275.75 | 60.0 |
| | (h*ng/mL) | | | | | |
| | AUC_{∞} | 2322 | 3980 | 171.38 | 106.50-275.79 | 59.8 |
| | (h*ng/mL) | | | | | |

N = 8 participants per cohort
Note:
Log transformed PK parameters were analyzed by an analysis of variance (ANOVA) model for Cohort as a factor and the results were back-transformed using anti-logarithm.
N: number of participants who completed at least one treatment and for which an evaluable PK parameter could be obtained were included.
% CV = 100* (sqrt(exp(MSE) − 1).

Based on the GMR of M12, mild hepatic impairment Cₘₐₓ and AUCs (AUCₗₐₛₜ and AUC_{∞}) were approximately higher by 1.11-fold and 1.71-fold, respectively, compared to normal hepatic function. The % CV is 31.3% for Cₘₐₓ, 60.0% and 59.8% for AUCs (AUCₗₐₛₜ, and AUC_{∞}). Point estimate ratios (90% CI) for Cₘₐₓ, AUCₗₐₛₜ, and AUC_{∞} of M12 for mild hepatic impairment versus normal hepatic function were 111.23% (85.48%-144.72%), 171.17% (106.25%-275.75%), and 171.38% (106.50%-275.79%), respectively.

Participants in Cohort 3 received a lower dose of 10 mg of seltorexant, therefore the PK parameters are dose normalized to 20 mg to be able to compare to the normal hepatic function (Cohort 1). A summary of statistical results of M12 for dose normalized moderate hepatic impairment versus normal hepatic function is presented in Table 14. Specifically, Table 14 provides summarized statistical results for estimated ratio of geometric means and 90% confidence interval of M12 exposure parameters after oral administration of 20 mg seltorexant in Cohort 1 and 10 mg seltorexant in Cohort 3 dose normalized to 20 mg.

TABLE 14

| | | | | Ratio of | | |
|---|---|---|---|---|---|---|
| | | | | Geometric | | |
| | PK | Cohort 1 | Cohort 3 | Means | 90% CI | |
| Analyte | Parameter | (Reference) | (Test) | (%) | (%) | % CV |
| M12 | Cₘₐₓ | 320 | 253 | 79.13 | 60.82-102.96 | 31.3 |
| | (ng/mL) | | | | | |
| | AUCₗₐₛₜ | 2310 | 2375 | 102.84 | 63.84-165.67 | 60.0 |
| | (h*ng/mL) | | | | | |
| | AUC_{∞} | 2322 | 2404 | 103.51 | 64.32-166.56 | 59.8 |
| | (h*ng/mL) | | | | | |

N = 8 participants per cohort
Note:
Log transformed PK parameters were analyzed by an analysis of variance (ANOVA) model for Cohort as a factor and the results were back-transformed using anti-logarithm.
N: number of participants who completed at least one treatment and for which an evaluable PK parameter could be obtained were included.
% CV = 100* (sqrt(exp(MSE) − 1).

Based on the GMR of M12, dose normalized moderate hepatic impairment Cₘₐₓ was 0.79-fold, while AUCₗₐₛₜ and AUC_{∞} were approximately higher by 1.03-fold and 1.04-fold, respectively, compared to normal hepatic function. The % CV is 31.3% for Cₘₐₓ, 60.0% and 59.8% for AUCs (AUCₗₐₛₜ and AUC_{∞}). Point estimate ratios (90% CI) for Cₘₐₓ, AUCₗₐₛₜ, and AUC_{∞} of M12 for dose normalized moderate hepatic impairment versus normal hepatic function were 79.13% (60.82%-102.96%), 102.84% (63.84%-165.67%) and 103.51% (64.32%-166.56%), respectively.

A summary of statistical results of unbound M12 for mild hepatic impairment versus normal hepatic function is presented in Table 15. Specifically, Table 15 shows summarized statistical results for estimated ratio of geometric means and 90% confidence interval of unbound M12 exposure parameters after oral administration of 20 mg seltorexant in Cohort 1 and Cohort 2.

TABLE 15

| | | | | Ratio of | | |
|---|---|---|---|---|---|---|
| | | | | Geometric | | |
| | PK | Cohort 1 | Cohort 2 | Means | 90% CI | |
| Analyte | Parameter | (Reference) | (Test) | (%) | (%) | % CV |
| UNBOUND | Cₘₐₓ | 16.3 | 21.0 | 128.83 | 92.51-179.42 | 40.0 |
| M12 | (ng/mL) | | | | | |
| | AUCₗₐₛₜ | 118 | 234 | 198.26 | 138.46-283.90 | 43.6 |
| | (h*ng/mL) | | | | | |
| | AUC_{∞} | 119 | 236 | 198.51 | 139.01-283.48 | 43.3 |
| | (h*ng/mL) | | | | | |

N = 8 participants per cohort
Note:
Log transformed PK parameters were analyzed by an analysis of variance (ANOVA) model for Cohort as a factor and the results were back-transformed using anti-logarithm.
N: number of participants who completed at least one treatment and for which an evaluable PK parameter could be obtained were included.
% CV = 100* (sqrt(exp(MSE) − 1).

Based on the GMR of unbound M12, mild hepatic impairment C_{max,unbound}, AUC_{last,unbound}, and AUC_{∞,unbound} were approximately higher by 1.29-fold, 1.98-fold, and 1.99-fold, respectively, compared to normal hepatic function. The % CV is 40.0% for C_{max,unbound}, 43.6% and 43.3% for AUCs (AUC_{last,unbound} and AUC_{∞,unbound}). Point estimate ratios (90% CI) for C_{max,unbound}, AUC_{last,unbound}, and AUC_{∞,unbound} of unbound M12 for mild hepatic impairment versus normal hepatic function were 128.83% (92.51%-179.42%), 198.26% (138.46%-283.90%) and 198.51% (139.01%-283.48%), respectively.

A summary of statistical results of unbound M12 for dose normalized moderate hepatic impairment versus normal hepatic function is presented in Table 16. Specifically, Table 16 provides summarized statistical results for estimated ratio of geometric means and 90% confidence interval of unbound M12 exposure parameters after oral administration of 20 mg seltorexant in Cohort 1 and 10 mg seltorexant in Cohort 3 (dose normalized to 20 mg).

TABLE 16

| | | | | Ratio of | | |
|---|---|---|---|---|---|---|
| | | | | Geometric | | |
| | PK | Cohort 1 | Cohort 3 | Means | 90% CI | |
| Analyte | Parameter | (Reference) | (Test) | (%) | (%) | % CV |
| UNBOUND | Cₘₐₓ | 16.3 | 27.5 | 168.44 | 120.95-234.58 | 40.0 |
| M12 | (ng/mL) | | | | | |
| | AUCₗₐₛₜ | 118 | 258 | 218.90 | 152.87-313.46 | 43.6 |
| | (h*ng/mL) | | | | | |
| | AUC_{∞} | 119 | 262 | 220.33 | 154.29-314.63 | 43.3 |
| | (h*ng/mL) | | | | | |

N = 8 participants per cohort
Note:
Log transformed PK parameters were analyzed by an analysis of variance (ANOVA) model for Cohort as a factor and the results were back-transformed using anti-logarithm.
N: number of participants who completed at least one treatment and for which an evaluable PK parameter could be obtained were included.
% CV = 100* (sqrt(exp(MSE) − 1).

Based on the GMR of unbound M12, dose normalized moderate hepatic impairment C_{max,unbound}, AUC_{last,unbound}, and AUC_{∞,unbound} were approximately higher by 1.68-fold, 2.19-fold, and 2.20-fold, respectively, compared to normal hepatic function. The % CV for C_{max,unbound}, AUC_{last,unbound}, and AUC_{∞,unbound} were 40.0%, 43.6%, and 43.3%, respectively. Point estimate ratios (90% CI) for C_{max,unbound}, AUC_{last,unbound}, and AUC_{∞,unbound} of unbound M12 for dose normalized moderate hepatic impairment versus normal hepatic function were 168.44% (120.95%-234.58%), 218.90% (152.87%-313.46%) and 220.33% (154.29%-314.63%), respectively.

### Pharmacokinetics of M16

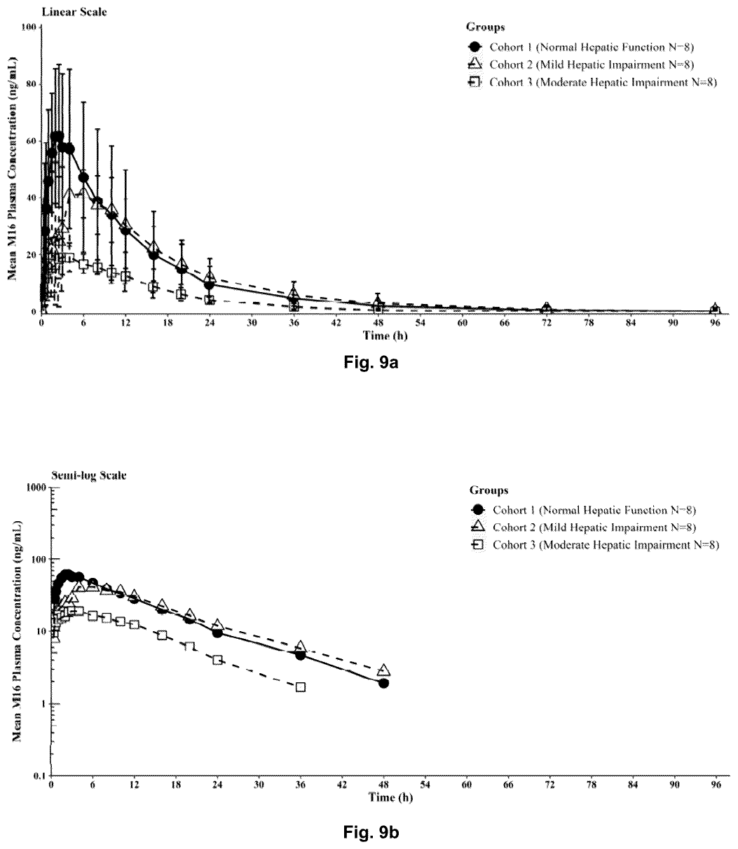

FIG. 9a shows the linear mean plasma concentration-time profiles of M16 after oral administration of 20 mg seltorexant in Cohort 1 and Cohort 2 and 10 mg seltorexant in Cohort 3. FIG. 9b shows the data of FIG. 9b in a semi-logarithmic scale. As shown in FIGS. 9a and 9b, following a single oral administration of 20 mg seltorexant (normal hepatic function and mild hepatic impairment) or 10 mg seltorexant (moderate hepatic impairment), the mean plasma concentration-time profiles of seltorexant declined mono-exponentially post reaching tₘₐₓ.

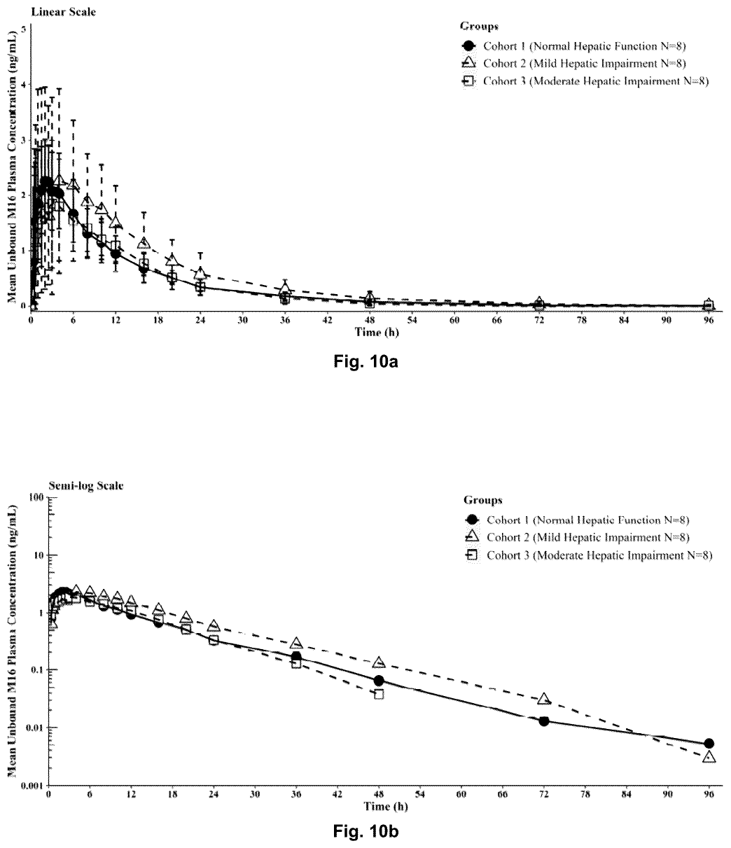

For all cohorts, 2-hours fraction unbound values were used to derive the unbound plasma concentrations and these unbound concentrations were used to derive unbound PK parameters. The mean fraction unbound values of M16 at 2-hours for normal hepatic function, mild hepatic impairment, and moderate hepatic impairment are 4.0%, 5.68% and 9.01%, respectively. FIG. 10a shows the linear mean plasma concentration-time profiles of unbound M16 after oral administration of 20 mg seltorexant in Cohort 1 and Cohort 2 and 10 mg seltorexant in Cohort 3. FIG. 10a shows the data of FIG. 10b in a semi-logarithmic scale. Following a single oral administration of 20 mg seltorexant (normal hepatic function and mild hepatic impairment) or 10 mg seltorexant (moderate hepatic impairment), the mean plasma concentration-time profiles of unbound M16 declined mono-exponentially post reaching tₘₐₓ.

A summary list of key PK parameters of M16 for all cohorts including dose normalized PK parameters is presented in Table 17.

TABLE 17

| Pharmaco- | | | | Moderate |
|---|---|---|---|---|
| kinetics | | | | Hepatic |
| parameter of | | | | Impairment |
| Seltorexant | Normal | | Moderate | Dose |
| (mean (SD), | Hepatic | Mild Hepatic | Hepatic | Normalized |
| tₘₐₓ:median | Function | Impairment | Impairment | (Cohort 3 dose |
| [Min-Max]) | (Cohort 1) | (Cohort 2) | (Cohort 3) | normalized) |
| n | 8 | 8 | 8 | 8 |
| Cₘₐₓ (ng/mL) | 69.0 (26.5) | 51.5 (13.4) | 21.5 (5.53) | 43.0 (11.1) |
| tₘₐₓ (h) | 2.00 | 4.00 [1.50- | 2.50 | — |
| | [0.33-4.00] | 10.00] | [0.67-8.00] | |
| AUCₗₐₛₜ | 885 (546) | 827 (304) | 315 (67.8) | 631 (136) |
| (h*ng/mL) | | | | |
| AUC_{∞} | 924 (571) | 851 (307) | 342 (64.8) | 683 (130) |
| (h*ng/mL) | | | | |
| λ_{z} (1/h) | 0.062 (0.020) | 0.076 (0.027) | 0.077 (0.030) | — |
| t_{1/2} (h) | 13.4 (8.53) | 10.2 (3.48) | 10.2 (3.86) | — |

Note:
Cohort 3 Dose Normalized to 20 mg

The mean M16 PK parameters Cₘₐₓ, AUCₗₐₛₜ, and AUC_{∞} for mild hepatic impairment (without dose normalization) are 51.5 ng/mL, 827 h*ng/ml and 851 h*ng/ml; for normal hepatic function are 69.0 ng/ml, 885 h*ng/ml and 924 h*ng/mL, respectively. The mild hepatic impairment mean PK parameter Cₘₐₓ was 0.75-fold, whereas AUCₗₐₛₜ and AUC_{∞} of M16 were comparable to normal hepatic function. Elimination half-life (t_{1/2}) and median tₘₐₓ of normal hepatic function, mild hepatic impairment, were 13.4 h, 10.2 h and 2.00 h, 4.00 h, respectively.

Participants in Cohort 3 (moderate hepatic impairment) received a lower dose of 10 mg of seltorexant, therefore the PK parameters are dose normalized to 20 mg to be able to compare to the normal hepatic function (Cohort 1). The mean M16 PK parameters Cₘₐₓ, AUCₗₐₛₜ, and AUC_{∞} for moderate hepatic impairment (without dose normalization) are 21.5 ng/ml, 315 h*ng/mL, and 342 h*ng/mL, respectively. The dose normalized moderate hepatic impairment mean PK parameters Cₘₐₓ, AUCₗₐₛₜ, and AUC_{∞} (43.0 ng/ml, 631 h*ng/ml and 683 h*ng/ml, respectively) of M16 were 0.62-, 0.71-, and 0.74-fold, compared to normal hepatic function (Cₘₐₓ, AUCₗₐₛₜ, and AUC_{∞} are 69.0 ng/ml, 885 h*ng/ml and 924 h*ng/mL, respectively). Elimination half-life (t_{1/2}) and median tₘₐₓ of normal hepatic function, moderate hepatic impairment were 13.4 h, 10.2 h and 2.00 h, 2.50 h, respectively.

Plasma protein binding of M16 in participants with varying degrees of hepatic function on Day 1 at 2 hours for plasma concentrations of α-1-AGP, total protein, and albumin, per cohort is presented in Table 18.

TABLE 18

| Plasma Protein | | | |
|---|---|---|---|
| Binding for Day 1- | Normal Hepatic | Mild Hepatic | Moderate Hepatic |
| 2 h of Metabolite | Function | Impairment | Impairment |
| M16 (mean (SD)) | (Cohort 1) | (Cohort 2) | (Cohort 3) |
| n | 8 | 8 | 8 |
| % fᵤ | 4.00 (1.43) | 5.68 (3.95) | 9.01 (3.14) |
| % f_{b} | 96.0 (1.43) | 94.3 (3.95) | 91.0 (3.14) |
| Albumin (g/dL) | 4.04 (0.182) | 3.94 (0.231) | 3.29 (0.380) |
| α-1-AGP (mg/dL) | 68.2 (23.6) | 54.4 (16.1) | 36.0 (9.16) |
| Total Protein (g/dL) | 6.38 (0.554) | 6.44 (0.391) | 5.98 (0.453) |

The relationships between albumin, bilirubin, prothrombin time, ALP, ALT, AST, total protein, and α-1-AGP to the M16 PK parameters (Cₘₐₓ, AUCₗₐₛₜ, and AUC_{∞}) were evaluated by fitting a linear regression model with the M16 PK parameters (Cₘₐₓ, AUCₗₐₛₜ, and AUC_{∞}) as the dependent variable and either albumin, bilirubin, prothrombin time, ALP, ALT, AST, total protein or α-1-AGP as a linear predictor (baseline values were used). The slope of the regression line for Cₘₐₓ, AUCₗₐₛₜ, and AUC_{∞} were found to be statistically significant for α-1-AGP (p-values were 0.0004, 0.0003 and 0.0003 respectively).

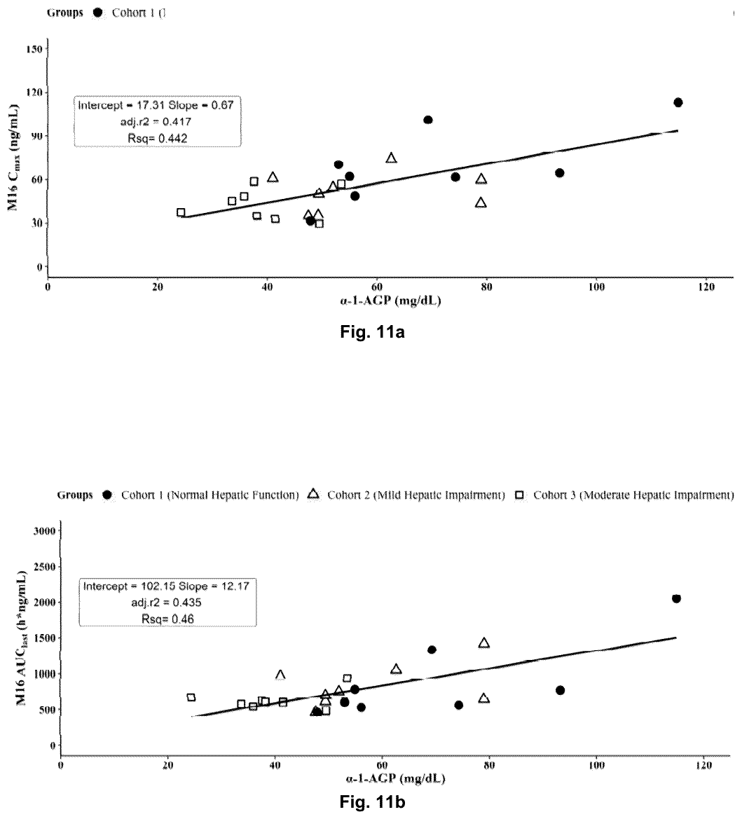

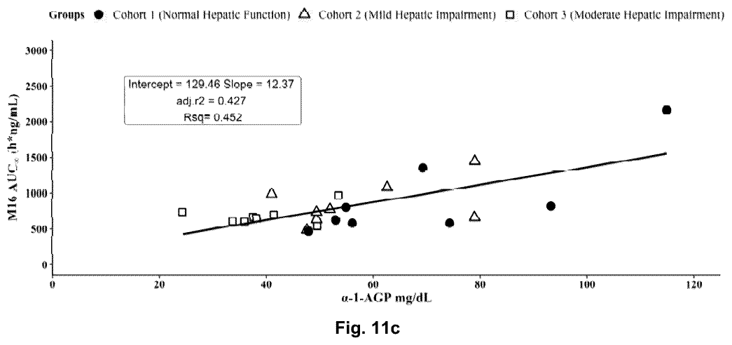

FIG. 11a shows a scatter plot of M16 Cₘₐₓ against α-1-AGP Levels after oral administration of 20 mg seltorexant in Cohort 1 and Cohort 2 and 10 mg seltorexant in Cohort 3 (dose normalized to 20 mg). FIG. 11b shows a scatter plot of M16 AUCₗₐₛₜ against α-1-AGP Levels after oral administration of 20 mg seltorexant in Cohort 1 and Cohort 2 and 10 mg seltorexant in Cohort 3 (dose normalized to 20 mg). FIG. 11c shows a scatter plot of M16 AUC_{∞} against α-1-AGP Levels after oral administration of 20 mg seltorexant in Cohort 1 and Cohort 2 and 10 mg seltorexant in Cohort 3 (dose normalized to 20 mg).

A summary list of key PK parameters of unbound M16 for all cohorts including dose normalized PK parameters are presented in Table 19.

TABLE 19

| Pharmaco- | | | | Moderate |
|---|---|---|---|---|
| kinetics | | | | Hepatic |
| parameter of | | | | Impairment |
| unbound | Normal | | Moderate | Dose |
| Metabolite M16 | Hepatic | Mild Hepatic | Hepatic | Normalized |
| (mean | Function | Impairment | Impairment | (Cohort 3 dose |
| (SD) | (Cohort 1) | (Cohort 2) | (Cohort 3) | normalized) |
| n | 8 | 8 | | 8 |
| Cₘₐₓ (ng/mL) | 2.55 (0.931) | 2.77 (1.73) | 2.01 (1.08) | 4.02 (2.16) |
| AUCₗₐₛₜ | 30.7 (10.5) | 41.9 (25.5) | 28.1 (9.99) | 56.1 (20.0) |
| (h*ng/mL) | | | | |
| AUC_{∞} | 31.9 (10.6) | 43.2 (26.2) | 30.4 (10.5) | 60.8 (21.0) |
| (h*ng/mL) | | | | |

Note:
Cohort 3 Dose Normalized to 20 mg

The mean PK parameters C_{max,unbound}, AUC_{last,unbound}, and AUC_{∞,unbound} for mild hepatic impairment are 2.77 ng/mL, 41.9 h*ng/mL, and 43.2 h*ng/ml; for normal hepatic function are 2.55 ng/ml, 30.7 h*ng/mL, and 31.9 h*ng/mL, respectively. The mild hepatic impairment mean PK parameters of C_{max,unbound} was comparable and AUC_{last,unbound} and AUC_{∞, unbound} of unbound M16 were higher by 1.36- and 1.35-fold, compared to normal hepatic function.

Participants in Cohort 3 (moderate hepatic impairment) received a lower dose of 10 mg of seltorexant, therefore the PK parameters are dose normalized to 20 mg to be able to compare to the normal hepatic function (Cohort 1). The mean PK parameters C_{max,unbound}, AUC_{last,unbound} and AUC_{∞,unbound} for moderate hepatic impairment (without dose normalization) are 2.01 ng/mL, 28.1 h*ng/mL, and 30.4 h*ng/mL, respectively. The dose normalized moderate hepatic impairment mean PK parameters C_{max,unbound}, AUC_{last,unbound} and AUC_{∞,unbound} (4.02 ng/mL, 56.1 h*ng/ml and 60.8 h*ng/mL, respectively) of unbound M16 were higher by 1.58-, 1.83-, and 1.91-fold, compared to normal hepatic function (C_{max,unbound}, AUC_{last,unbound} and AUC_{∞, unbound} are 2.55 ng/ml, 30.7 h*ng/ml, and 31.9 h*ng/ml, respectively).

The relationships between albumin, bilirubin, prothrombin time, ALP, ALT, AST, total protein, and α-1-AGP to the unbound M16 PK parameters (C_{max,unbound}, AUCₗₐₛₜ, unbound, and AUC_{∞,unbound}) were evaluated by fitting a linear regression model with the unbound M16 PK parameters (C_{max,unbound}, AUC_{last,unbound} and AUC_{∞,unbound}) as the dependent variable and cither albumin, bilirubin, prothrombin time, ALP, ALT, AST, total protein, or α-1-AGP as a linear predictor (baseline values were used). The slope of the regression line for AUC_{last,unbound} and AUC_{∞,unbound} were found to be statistically significant for albumin, prothrombin time, α-1-AGP (p-values were 0.0465, 0.0190, 0.0041, and 0.0295, 0.0158, 0.0026, respectively) and C_{max, unbound} was found to be statistically significant for both prothrombin time and α-1-AGP (p-values were 0.0169 and 0.0077, respectively).

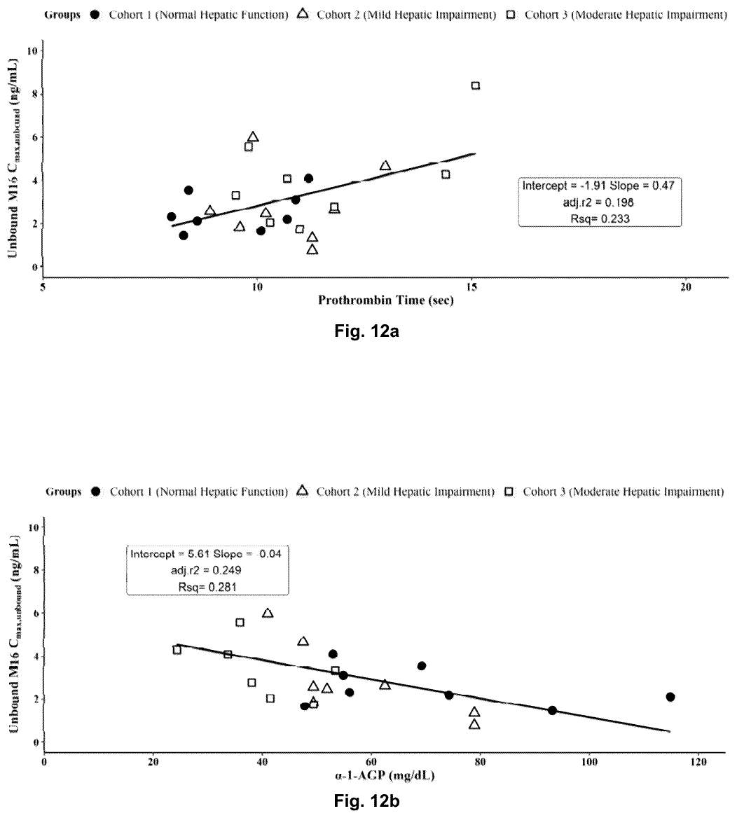

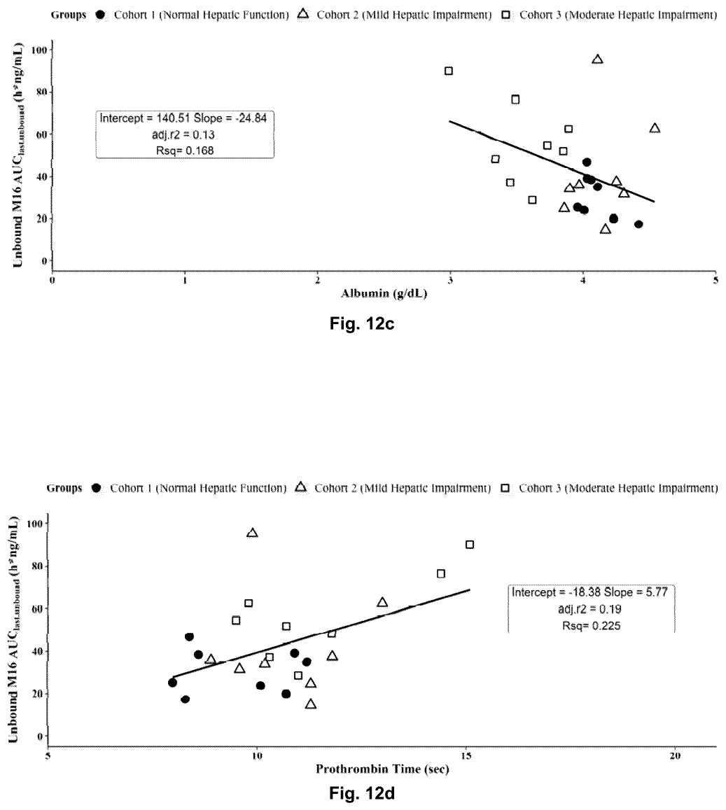

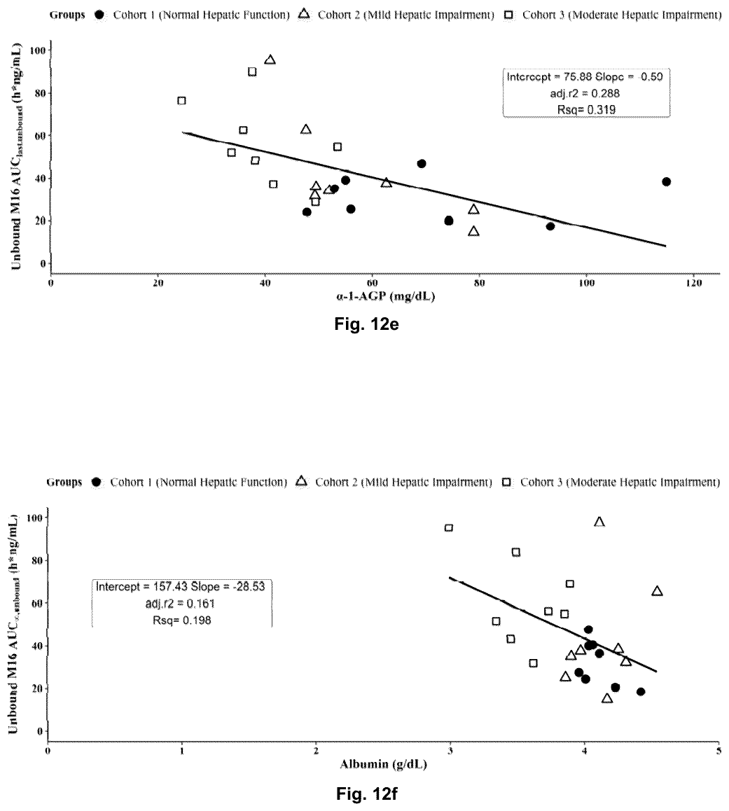

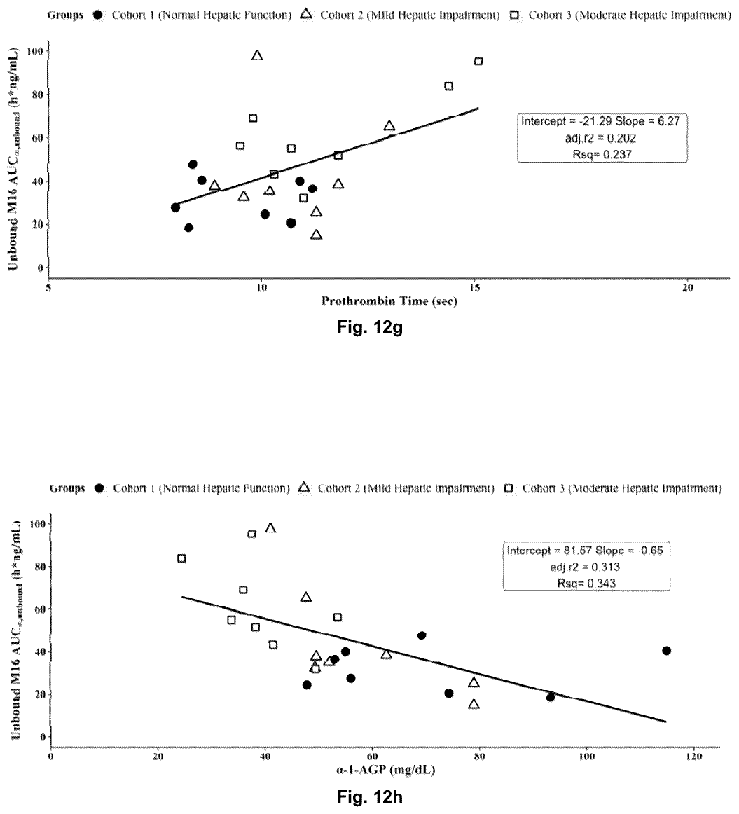

FIG. 12a shows a scatter plot of unbound M16 C_{max,unbound} against prothrombin time after oral administration of 20 mg seltorexant in Cohort 1 and Cohort 2 and 10 mg seltorexant in Cohort 3 (dose normalized to 20 mg). FIG. 12b shows a scatter plot of unbound M16 C_{max,unbound} against α-1-AGP levels after oral administration of 20 mg seltorexant in Cohort 1 and Cohort 2 and 10 mg seltorexant in Cohort 3 (dose normalized to 20 mg). FIG. 12c shows a scatter plot of unbound M16 AUC_{last,unbound} against albumin levels after oral administration of 20 mg seltorexant in Cohort 1 and Cohort 2 and 10 mg seltorexant in Cohort 3 (dose normalized to 20 mg). FIG. 12d shows a scatter plot of unbound M16 AUC_{last,unbound} against prothrombin time after oral administration of 20 mg seltorexant in Cohort 1 and Cohort 2 and 10 mg seltorexant in Cohort 3 (dose normalized to 20 mg). FIG. 12e shows a scatter plot of unbound M16 AUC_{last, unbound} against α-1-AGP levels after oral administration of 20 mg seltorexant in Cohort 1 and Cohort 2 and 10 mg seltorexant in Cohort 3 (dose normalized to 20 mg). FIG. 12f shows a scatter plot of unbound M16 AUC_{last,unbound} against albumin levels after oral administration of 20 mg seltorexant in Cohort 1 and Cohort 2 and 10 mg seltorexant in Cohort 3 (dose normalized to 20 mg). FIG. 12g shows a scatter plot of unbound M16 AUC_{∞,unbound} against prothrombin time after oral administration of 20 mg seltorexant in Cohort 1 and Cohort 2 and 10 mg seltorexant in Cohort 3 (dose normalized to 20 mg). FIG. 12h shows a scatter plot of unbound M16 AUC_{∞, unbound} against α-1-AGP levels after oral administration of 20 mg seltorexant in Cohort 1 and Cohort 2 and 10 mg seltorexant in Cohort 3 (dose normalized to 20 mg).

A summary of statistical results of M16 for mild hepatic impairment versus normal hepatic function is presented in Table 20. Specifically, Table 20 shows summarized statistical results for estimated ratio of geometric means and 90% confidence interval of M16 exposure parameters after oral administration of 20 mg seltorexant in Cohort 1 and Cohort 2.

TABLE 20

| | | | | Ratio of | | |
|---|---|---|---|---|---|---|
| | | | | Geometric | | |
| | PK | Cohort 1 | Cohort 2 | Means | 90% CI | |
| Analyte | Parameter | (Reference) | (Test) | (%) | (%) | % CV |
| M16 | Cₘₐₓ | 64.6 | 50.0 | 77.40 | 59.08-101.39 | 32.2 |
| | (ng/mL) | | | | | |
| | AUCₗₐₛₜ | 778 | 783 | 100.62 | 72.87-138.94 | 38.9 |
| | (h*ng/mL) | | | | | |
| | AUC_{∞} | 812 | 807 | 99.38 | 72.35-136.51 | 38.2 |
| | (h*ng/mL) | | | | | |

N = 8 participants per cohort
Note:
Log transformed PK parameters were analyzed by an analysis of variance (ANOVA) model for Cohort as a factor and the results were back-transformed using anti-logarithm.
N: number of participants who completed at least one treatment and for which an evaluable PK parameter could be obtained were included.
% CV = 100* (sqrt(exp(MSE) − 1).

Based on the GMR of M16, mild hepatic impairment Cₘₐₓ, AUCₗₐₛₜ, and AUC_{∞} were approximately 0.77-fold, 1.01-fold, and 0.99-fold, respectively, compared to normal hepatic function. The % CV is 32.2% for Cₘₐₓ, 38.9% and 38.2% for AUCs (AUC and AUC_{∞}). Point estimate ratios (90% CI) for Cₘₐₓ, AUCₗₐₛₜ, and AUC_{∞} of M16 for mild hepatic impairment versus normal hepatic function were 77.40% (59.08%-101.39%), 100.62% (72.87%-138.94%) and 99.38% (72.35%-136.51%), respectively.

Participants in Cohort 3 received a lower dose of 10 mg of seltorexant, therefore the PK parameters are dose normalized to 20 mg to be able to compare to the normal hepatic function (Cohort 1). A summary of statistical results of M16 for dose normalized moderate hepatic impairment versus normal hepatic function is presented in Table 21.

TABLE 21

| | | | | Ratio of | | |
|---|---|---|---|---|---|---|
| | | | | Geometric | | |
| | PK | Cohort 1 | Cohort 3 | Means | 90% CI | |
| Analyte | Parameter | (Reference) | (Test) | (%) | (%) | % CV |
| M16 | Cₘₐₓ | 64.6 | 41.8 | 64.69 | 49.38-84.74 | 32.2 |
| | (ng/mL) | | | | | |
| | AUCₗₐₛₜ | 778 | 620 | 79.68 | 57.71-110.03 | 38.9 |
| | (h*ng/mL) | | | | | |
| | AUC_{∞} | 812 | 674 | 83.00 | 60.42-114.01 | 38.2 |
| | (h*ng/mL) | | | | | |

N = 8 participants per cohort
Note:
Log transformed PK parameters were analyzed by an analysis of variance (ANOVA) model for Cohort as a factor and the results were back-transformed using anti-logarithm.
N: number of participants who completed at least one treatment and for which an evaluable PK parameter could be obtained were included.
% CV = 100* (sqrt(exp(MSE) − 1).

Based on the GMR of M16, dose normalized moderate hepatic impairment Cₘₐₓ, AUCₗₐₛₜ, and AUC_{∞} were approximately 0.65-fold, 0.80-fold, and 0.83-fold, respectively, compared to normal hepatic function. The % CV is 32.2% for Cₘₐₓ, 38.9% and 38.2% for AUCs (AUCₗₐₛₜ and AUC_{∞}). Point estimate ratios (90% CI) for Cₘₐₓ, AUCₗₐₛₜ, and AUC_{∞} of M16 for dose normalized moderate hepatic impairment versus normal hepatic function were 64.69% (49.38%-84.74%), 79.68% (57.71%-110.03%), and 83.00% (60.42%-114.01%), respectively.

The summary of statistical results of unbound M16 for mild hepatic impairment versus normal hepatic function is presented in Table 22. Specifically, Table 22 provides summarized statistical results for estimated ratio of geometric means and 90% confidence interval of unbound M16 exposure parameters after oral administration of 20 mg seltorexant in Cohort 1 and Cohort 2.

TABLE 22

| | | | | Ratio of | | |
|---|---|---|---|---|---|---|
| | | | | Geometric | | |
| | PK | Cohort 1 | Cohort 2 | Means | 90% CI | |
| Analyte | Parameter | (Reference) | (Test) | (%) | (%) | % CV |
| UNBOUND | Cₘₐₓ | 2.41 | 2.32 | 96.12 | 61.06-151.31 | 56.6 |
| M16 | (ng/mL) | | | | | |
| | AUCₗₐₛₜ | 29.0 | 36.3 | 124.96 | 85.37-182.90 | 46.5 |
| | (h*ng/mL) | | | | | |
| | AUC_{∞} | 30.3 | 37.4 | 123.42 | 84.74-179.76 | 45.9 |
| | (h*ng/mL) | | | | | |

N = 8 participants per cohort
Note:
Log transformed PK parameters were analyzed by an analysis of variance (ANOVA) model for Cohort as a factor and the results were back-transformed using anti-logarithm.
N: number of participants who completed at least one treatment and for which an evaluable PK parameter could be obtained were included.
% CV = 100* (sqrt(exp(MSE) − 1).

Based on the GMR of unbound M16, mild hepatic impairment C_{max,unbound} was 0.96-fold, AUC_{last,unbound} and AUC_{∞,unbound} were approximately higher by 1.25-fold and 1.23-fold, respectively, compared to normal hepatic function. The % CV is 56.6% for C_{max,unbound}, 46.5% and 45.9% for AUCs (AUC_{last,unbound} and AUC_{∞,unbound}). Point estimate ratios (90% CI) for C_{max,unbound}, AUC_{last,unbound} and AUC_{∞,unbound} of unbound M16 for mild hepatic impairment versus normal hepatic function were 96.12% (61.06%-151.31%), 124.96% (85.37%-182.90%), and 123.42% (84.74%-179.76%), respectively.

A summary of statistical results of unbound M16 for dose normalized moderate hepatic impairment versus normal hepatic function is presented in Table 23. Specifically, Table 23 provides summarized statistical results for estimated ratio of geometric means and 90% confidence interval of unbound M16 exposure parameters after oral administration of 20 mg seltorexant in Cohort 1 and 10 mg Seltorexant in Cohort 3 (dose normalized to 20 mg);

TABLE 23

| | | | | Ratio of | | |
|---|---|---|---|---|---|---|
| | | | | Geometric | | |
| | PK | Cohort 1 | Cohort 3 | Means | 90% CI | |
| Analyte | Parameter | (Reference) | (Test) | (%) | (%) | % CV |
| UNBOUND | Cₘₐₓ | 2.41 | 3.57 | 148.13 | 94.10-233.18 | 56.6 |
| M16 | (ng/mL) | | | | | |
| | AUCₗₐₛₜ | 29.0 | 53.0 | 182.46 | 124.66-267.08 | 46.5 |
| | (h*ng/mL) | | | | | |
| | AUC_{∞} | 30.3 | 57.6 | 190.05 | 130.49-276.81 | 45.9 |
| | (h*ng/mL) | | | | | |

N = 8 participants per cohort
Note:
Log transformed PK parameters were analyzed by an analysis of variance (ANOVA) model for Cohort as a factor and the results were back-transformed using anti-logarithm.
N: number of participants who completed at least one treatment and for which an evaluable PK parameter could be obtained were included.
% CV = 100* (sqrt(exp(MSE) − 1).

Based on the GMR of unbound M16, dose normalized moderate hepatic impairment C_{max,unbound}, AUC_{last,unbound} and AUC_{∞,unbound} were approximately higher by 1.48-fold, 1.82-fold, and 1.90-fold, respectively, compared to normal hepatic function. The % CV for C_{max,unbound}, AUC_{last,unbound}, and AUC_{∞,unbound} were 56.6%, 46.5% and 45.9%, respectively. Point estimate ratios (90% CI) for C_{max,unbound}, AUC_{last,unbound} and AUC_{∞,unbound} of unbound M16 for dose normalized moderate hepatic impairment versus normal hepatic function were 148.13% (94.10%-233.18%), 182.46% (124.66%-267.08%) and 190.05% (130.49%-276.81%), respectively.

The invention described and claimed herein is not to be limited in scope by the specific embodiments herein disclosed since these embodiments are intended as illustrations of several aspects of this invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. All publications cited herein are incorporated by reference in their entirety.

What is claimed is:

1. A method of treating an orexin-2 mediated disorder in a human subject in need thereof, the subject having moderate hepatic impairment, the method comprising:
administering seltorexant or a pharmaceutically acceptable salt or hydrate thereof at a reduced dose that is less than a therapeutically effective daily dose adapted for treatment of a human subject having normal hepatic function.

2. A method of treating an orexin-2 mediated disorder in a human subject in need thereof, the method comprising:
if the subject has moderate hepatic impairment, administering seltorexant or a pharmaceutically acceptable salt or hydrate thereof at a reduced dose that is less than a therapeutically effective daily dose adapted for treatment of a human subject having normal hepatic function;
if the subject has severe hepatic impairment, not administering seltorexant or a pharmaceutically acceptable salt or hydrate thereof to the subject; and
if the subject does not have moderate to severe hepatic impairment, administering seltorexant or a pharmaceutically acceptable salt or hydrate thereof at the daily dose to the subject.

3. The method of claim 1, wherein the subject having the moderate hepatic impairment has a Child-Pugh score from 7 to 9.

4. The method of claim 1, wherein seltorexant is administered as a free base.

5. The method of claim 1, wherein a hydrochloride salt of seltorexant is administered.

6. The method of claim 1, wherein the orexin-2 mediated disorder is depression or insomnia.

7. The method of claim 6, wherein the orexin-2 mediated disorder is depression.

8. The method of claim 7, wherein the depression is major depressive disorder.

9. The method of claim 7, wherein the depression is bipolar depression.

10. The method of claim 8, wherein the subject has concurrent insomnia symptoms.

11. The method of claim 10, wherein the subject has an ISI score of greater than or equal to 15.

12. The method of claim 6, wherein the orexin-2 mediated disorder is insomnia.

13. The method of claim 8, wherein seltorexant is orally administered.

14. The method of claim 13, wherein the daily dose is about 20 mg/day to about 60 mg/day.

15. The method of claim 14, wherein the reduced dose is about 5 mg/day to about 20 mg/day.

16. The method of claim 15, wherein the reduced dose is about 20 mg/day.

17. The method of claim 15, wherein the reduced dose is about 15 mg/day.

18. The method of claim 15, wherein the reduced dose is about 10 mg/day.

19. The method of claim 15, wherein the reduced dose is about 5 mg/day.

20. The method of claim 14, wherein the daily dose is selected to provide an unbound average plasma concentration of seltorexant between about 0.75 ng/ml and about 1.5 ng/ml when administered the human subject having normal hepatic function.

21. The method of claim 20, wherein administration of seltorexant or the pharmaceutically acceptable salt or hydrate thereof at the reduced dose to the subject having moderate hepatic impairment provides an unbound average plasma concentration of seltorexant within an effective therapeutic range.

22. The method of claim 14, wherein the subject had an inadequate response to an antidepressant other than seltorexant or a pharmaceutically acceptable salt or hydrate thereof.

23. The method of claim 22, wherein the subject had an inadequate response to a selective serotonin reuptake inhibitor (SSRI) or a serotonin and noradrenaline reuptake inhibitor (SNRI).

24. The method of claim 23, wherein seltorexant or a pharmaceutically acceptable salt or hydrate thereof is adjunctively administered with a second pharmaceutically active agent.

25. The method of claim 24, wherein the second pharmaceutically active agent is a selective serotonin reuptake inhibitor (SSRI) or a serotonin and noradrenaline reuptake inhibitor (SNRI).

26. The method of claim 2, wherein the subject having the mild hepatic impairment has a Child-Pugh score from 5 to 6.

27. The method of claim 2, wherein the subject having the severe hepatic impairment has a Child-Pugh score at or above 10.

28. The method of claim 2, wherein the orexin-2 mediated disorder is major depressive disorder.

29. The method of claim 28, wherein the subject has concurrent insomnia symptoms.

30. The method of claim 28, wherein the daily dose is about 20 mg/day to about 60 mg/day.
